(19) United States
(12) Patent Application Publication
(10) Pub. No.: US 2012/0022367 A1
(43) Pub. Date: Jan. 26, 2012

(54) Chemically-selective, label free, microendoscopic system based on coherent anti-stokes raman scattering and microelectromechanical fiber optic probe

(76) Inventors: Stephen T.C. Wong, Missouri City, TX (US); Zhiyong Wang, Sugar Land, TX (US); Ganesh Palpattu, Houston, TX (US); Liang Gao, Houston, TX (US)

(21) Appl. No.: 13/135,737

(22) Filed: Jul. 14, 2011

Related U.S. Application Data
(63) Continuation of application No. 12/931,142, filed on Jan. 25, 2011.
Application No. 12/931,142 is a continuation-in-part of application No. PCT/US11/43729, filed on Jul. 12, 2011.
(60) Provisional application No. 61/399,139, filed on Jul. 8, 2010.
(60) Provisional application No. 61/399,182, filed on Jul. 8, 2010.

(51) Int. Cl. A61B 6/00; A61B 18/18

(52) U.S. Cl. 600/427

(57) ABSTRACT

An endoscopic microscopic system for collecting and processing a sequence of images in order to provide diagnosis and treatment.

### 1. CROSS REFERENCE TO RELATED APPLICATIONS

This application is a continuation in part of U.S. utility patent application Ser. No. 12/931,142, attorney docket no. 058001.105020, filed on Jan. 25, 2011, which claims priority to U.S. Provisional Patent Application Nos. 61/399,182 and 61/399,139, all of which are incorporated herein by reference.

This application is a continuation in part of PCT patent application serial no. PCT/US11/43792, attorney docket no. 058001.105020, filed on Jul. 12, 2011, which claims priority to U.S. utility patent application Ser. No. 12/931,142 as well as U.S. Provisional Patent Application Nos. 61/399,182 and 61/399,139, all of which are incorporated herein by reference.

The application claims the benefit of the filing date of U.S. patent application No. 61/399,182, attorney docket no. 058001.105021-PROV, filed on Jul. 8, 2010, the disclosure of which is incorporated herein by reference.

The application claims the benefit of the filing date of U.S. patent application No. 61/399,139, attorney docket no. 058001.105020-PROV, filed on Jul. 8, 2010, the disclosure of which is incorporated herein by reference.

### 2. BACKGROUND

This disclosure relates to microendoscopic systems used for diagnosis and treatment of patients.

### BRIEF DESCRIPTION OF THE DRAWINGS

FIG. 1 is an illustration of an exemplary embodiment of a micro-endoscopic system.

FIG. 2 is an illustration of an exemplary embodiment of a micro-endoscopic system.

FIG. 3 is an illustration of an exemplary embodiment of a micro-endoscopic system.

FIG. 3a is a schematic illustration of an exemplary embodiment of a wave division multiplexer assembly.

FIG. 3b is a schematic illustration of an exemplary embodiment of a wave division multiplexer assembly.

FIG. 3c is a schematic illustration of an exemplary embodiment of a fiber probe system.

FIG. 4 is a schematic illustration of an exemplary embodiment of a micro-endoscopic system.

FIG. 5 is a flow chart of an exemplary embodiment of a method for operating a micro-endoscopic system.

FIG. 6 is a flow chart of an exemplary embodiment of a method for calculating a global registration.

FIG. 7 is a photograph of a reference image.

FIGS. 7a-7e are simulated images derived from the reference image of FIG. 7.

FIGS. 8a-8e are motion corrected images resulting from the application of an exemplary experimental embodiment of the method of FIG. 6 to the simulated images of FIGS. 7a-7e.

FIGS. 9a-9e are the difference between the reference image of FIG. 7 and the motion corrected images of FIGS. 8a-8e.

FIG. 10 is a graphical illustration of the error for the images generated in an exemplary experimental embodiment using the motion correction method of FIG. 6 versus the error for images generated using a conventional NMI method of motion correction.

FIG. 11 is a photograph of a reference image in an exemplary experimental embodiment.

FIGS. 11a-11d are photographs of exemplary experimental embodiments.

FIGS. 12a-12d are photographs of exemplary experimental embodiments.

FIG. 13 is a flow chart of an exemplary embodiment of a method for calculating a global registration.

FIG. 14 is a flow chart of an exemplary embodiment of a method for calculating a global registration.

FIGS. 15a and 15b are photographs of exemplary experimental embodiments.

FIG. 16 is a photograph of a reference image in an exemplary experimental embodiment.

FIGS. 16a-16d are photographs of exemplary experimental embodiments.

FIGS. 17a-17d are graphical illustrations of exemplary experimental embodiments.

FIG. 18 is a graphical illustration of exemplary experimental embodiments.

FIGS. 19a and 19b is a flow chart of an exemplary embodiment of a method for motion correction.

FIG. 20 is an illustration of exemplary experimental embodiments.

FIG. 21 is an illustration of exemplary experimental embodiments.

FIG. 22 is a schematic illustration of an exemplary embodiment of a method for motion correction.

FIGS. 23a-23c are illustrations of exemplary experimental embodiments.

FIGS. 24a-24c are illustrations of exemplary experimental embodiments.

FIGS. 25a-25c are graphical illustrations of exemplary experimental embodiments.

FIGS. 26a and 26b is a schematic illustration of an exemplary embodiment of a CARS microscopy system.

FIGS. 27a, 27b and 27c are graphical illustrations of an exemplary experimental embodiment of the system of FIGS. 26a and 26b.

FIGS. 28a and 28b are graphical illustrations of an exemplary experimental embodiment of the system of FIGS. 26a and 26b.

FIGS. 30a, 30b, 30c, 30d, 30e and 30f are images of exemplary experimental embodiments of the system of FIGS. 26a and 26b.

FIGS. 31a, 31b, 31c, and 31d are images of exemplary experimental embodiments of the system of FIGS. 26a and 26b.

FIG. 32 is a schematic illustration of an exemplary embodiment of a CARS microscopy system.

FIG. 33 is a schematic illustration of an exemplary embodiment of a scanning mirror for the CARS microscopy system of FIG. 32.

FIG. 34 is a schematic illustration of an exemplary embodiment of a scanning mirror for the CARS microscopy system of FIG. 32.

FIG. 35 is a schematic illustration of an exemplary experimental embodiment of a CARS microscopy system.

FIG. 36 is a schematic illustration of an exemplary embodiment of a minimally invasive multimodality image guided molecular imaging system for treatment and diagnosis of patients.

FIG. 37 is a schematic illustration of an exemplary embodiment of a minimally invasive multimodality image guided molecular imaging system for treatment and diagnosis of patients.

FIG. 38 is a flow chart of an exemplary embodiment of a method of operating the systems of FIGS. 36 and 37.

FIG. 39 is a flow chart illustration of an exemplary method of operating the systems of FIGS. 36 and 37.

FIGS. 39a, 39b and 39c are illustrations of exemplary user interfaces provided by the method of FIG. 39.

FIG. 39d is a schematic illustration of an exemplary embodiment of a respiratory motion correction method.

FIG. 40 is an illustration of an exemplary embodiment of a user interface during exemplary experimental embodiments of the system and method of FIGS. 26 and 27.

FIG. 41 is an illustration of an exemplary embodiment of a user interface during exemplary experimental embodiments of the system and method of FIGS. 26 and 27.

FIGS. 42a, 42b and 42c are illustrations of exemplary embodiments of user interfaces during exemplary experimental embodiments of the system and methods of FIGS. 36, 37, 38 and 39.

FIGS. 43a, 43b, 43c and 43d are illustrations of exemplary embodiments of user interfaces during exemplary experimental embodiments of the system and methods of FIGS. 36, 37, 38 and 39.

FIGS. 44a, 44b, 44c and 44d are illustrations of exemplary embodiments of user interfaces during exemplary experimental embodiments of the system and methods of FIGS. 36, 37, 38 and 39.

FIGS. 45a, 45b, 45c and 45d are images of exemplary experimental embodiments of the system and methods of FIGS. 36, 37, 38 and 39.

FIG. 46 is a graphical illustration of exemplary experimental embodiments of the system and methods of FIGS. 36, 37, 38 and 39.

FIG. 47 are images of exemplary experimental embodiments of the system and methods of FIGS. 36, 37, 38 and 39.

FIG. 48 are images of exemplary experimental embodiments of the system and methods of FIGS. 36, 37, 38 and 39.

FIGS. 49a, 49b, 49c and 49d are images of exemplary experimental embodiments of the system and methods of FIGS. 36, 37, 38 and 39.

FIGS. 50a, 50b, 50c and 50d are images of exemplary experimental embodiments of the system and methods of FIGS. 36, 37, 38 and 39.

FIG. 51 is a graphical illustration of exemplary experimental embodiments of the system and methods of FIGS. 36, 37, 38 and 39.

FIGS. 52a and 52b are graphical illustrations of exemplary experimental embodiments of the system and methods of FIGS. 36, 37, 38 and 39.

FIGS. 53a, 53b and 53c are graphical illustrations of exemplary experimental embodiments of the system and methods of FIGS. 36, 37, 38 and 39.

FIG. 54 is an illustration of exemplary experimental embodiments of the system and methods of FIGS. 36, 37, 38 and 39.

FIGS. 55a and 55b are illustrations of exemplary experimental embodiments of the system and methods of FIGS. 36, 37, 38 and 39.

FIGS. 56a, 56b and 56c are illustrations of exemplary experimental embodiments of the system and methods of FIGS. 36, 37, 38 and 39.

### DETAILED DESCRIPTION

In the drawings and description that follows, like parts are marked throughout the specification and drawings with the same reference numerals, respectively. The drawings are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results. The various characteristics mentioned above, as well as other features and characteristics described in more detail below, will be readily apparent to those skilled in the art upon reading the following detailed description of the embodiments, and by referring to the accompanying drawings.

As a nonlinear optical imaging technique, coherent anti-Stokes Raman scattering (“CARS”) imaging has been demonstrated as a powerful tool for label-free optical imaging. This technique offers many advantages including (a) chemically selective contrasts based on Raman vibrational activity, (b) high sensitivity and rapid acquisition rates due to the coherent nature of the CARS process, (c) and sub-wavelength spatial resolution. Because of its highly-directional coherent property, the CARS signal is several orders of magnitude stronger than the conventional Raman signal; therefore, CARS offers ultrafast imaging capability in video rate in vivo. In addition, the CARS signal is generated only at the laser focus, enabling point-by-point three-dimensional imaging for 3D sectioning without a confocal aperture. As a result, CARS microscopy has been successfully applied to imaging viruses, cells, tissues, and live animals, using signals from CH₂-abundant structures. CARS utilizes two laser beams. A pump beam at frequency ωₚ and a Stokes beam at frequency ωₛ (where ωₛ<ωₚ) tightly focus onto the sample, resulting in an emission signal at the anti-Stokes frequency (ω_{CARS}=2ωₚ−ωₛ). For spectroscopic or multiplex CARS applications, a femtosecond laser is often used as the Stokes wave by taking advantage of its broad spectral band to cover the spectral range of interest. For narrowband CARS imaging applications, picosecond (“ps”) lasers are typically used due to the reduction of the non-resonant CARS background, the excitation efficiency and the spectral resolution.

Single-mode fibers (“SMF”) and single-mode photonic crystal fibers (“PCF”) have been successfully demonstrated for use in the CARS imaging system. However, in order to satisfy the single-mode condition, the core diameter of the step-index SMF is typically limited to ˜5 μm. Relatively small-sized core can make SMF susceptible to generating nonlinear effects, e.g. self-phase modulation (“SPM”), which may reshape the spectra of laser pulses. Although the core diameter of PCF is larger than SMF (e.g. ˜16 μm for large mode area PCF), the numerical aperture (“NA”) of PCF is usually small (e.g. ˜0.06 for large mode area PCF) and, thus, results in difficulty and instability of laser coupling as well as small coupling efficiencies (<30%). This, in order to address these issues, in the present exemplary embodiments, multimode fibers (“MMF”) may be used for delivery of ultrafast pulses for CARS imaging. Compared to SMFs and PCFs, step-index MMFs have larger core diameters, larger NA, and larger coupling efficiency. In spite of these advantages, the delivery of two ultrafast pulses in the multimode fiber for CARS imaging has not been investigated.

Furthermore, in the exemplary embodiments, we may use standard commercial MMF, e.g., Corning SMF28 fibers, to deliver picosecond excitation lasers for CARS imaging. Furthermore, in several exemplary experimental embodiments, we experimentally analyzed issues associated with the fiber delivery, such as, for example, dispersion length, walk-off length, nonlinear length, average threshold power for self-phase modulations, and four-wave mixing (“FWM”). The exemplary experimental embodiments demonstrated that FWM signals are generated in MMF, but they can be filtered out by a long-pass filter for CARS imaging. Finally, we further demonstrated, in the exemplary experimental embodiments, that MMF can be used for delivery of picosecond excitation lasers without any degradation of CARS image quality.

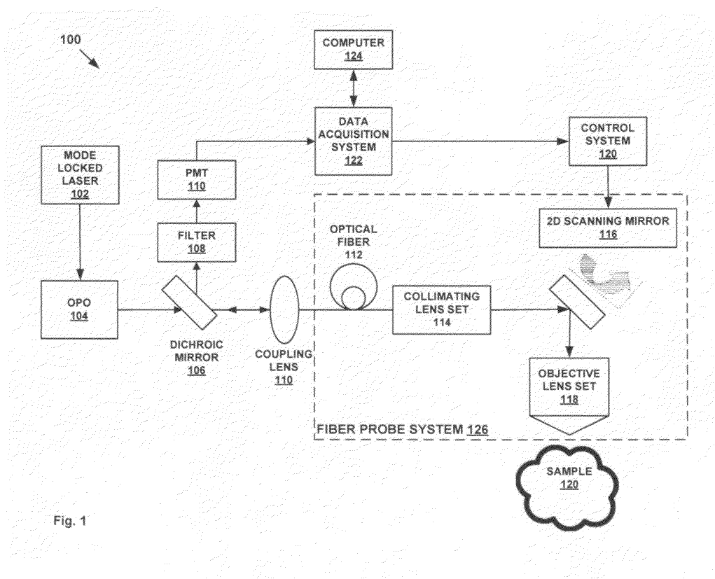

Referring initially to FIG. 1, a micro-endoscopic system 100 includes a mode locked laser 102 having an output that is operably coupled to an optical parametric oscillator (“OPO”) 104. The output of the OPO 104 is operably coupled to an input of a dichroic mirror 106, an output of the dichroic mirror 106 is operably coupled to an input of an optical filter 108, and an input/output of the dichroic mirror is operably coupled to coupling lens 110. The output of the optical filter 108 is operably coupled to an input of a photomultiplier tube (“PMT”) 110. The coupling lens 110 is also operably coupled to an end of an optical fiber 112 and the other end of the optical fiber is operably coupled to a collimating lens set 114. The collimating lens set 114 is also operably coupled to a 2D scanning mirror 116 and the 2D scanning mirror is also operably coupled to an objective lens set 118 that may be positioned proximate a sample. The 2D scanning mirror is further operably coupled to a control system 120 for controlling and monitoring the operation of the 2D scanning mirror. A data acquisition system 122 is operably coupled to the PMT 110 and the control system 120 for monitoring and controlling the operation of the PMT and the control system. A computer 124 is operably coupled to the data acquisition system 122 for monitoring and controlling the operation of the data acquisition system.

In an exemplary embodiment, during the operation of the system 100, the system is operated to provide CARS microscopy which provides for the imaging of chemical and biological samples by using molecular vibrations as a contrast mechanism. In particular, as will be recognized by persons having ordinary skill in the art, CARS microscopy uses at least two laser fields, a pump electromagnetic field with a center frequency at ωₚ and a Stokes electromagnetic field with a center frequency at ωₛ. The pump and Stokes fields interact with a sample and generate a coherent anti-Stokes field having a frequency of ω_{AS}=2ωₚ−ωₛ in the phase matched direction. When the Raman shift of ωₚ−ωₛ is tuned to be resonant at a given vibrational mode, an enhanced CARS signal is observed at the anti-Stokes frequency ω_{AS}. Unlike fluorescence microscopy, CARS microscopy does not require the use of fluorophores (which may undergo photobleaching), since the imaging relies on vibrational contrast of biological and chemical materials. Further, the coherent nature of CARS microscopy offers significantly higher sensitivity than spontaneous Raman microscopy. This permits the use of lower average excitation powers (which is tolerable for biological samples). The fact that ω_{AS}>ωₚ, ω_{S} allows the signal to be detected in the presence of background fluorescence.

In particular, in an exemplary embodiment, during the operation of the system 100, the mode locked laser 102 and the OPO 104 are operated in a well known manner to generate a pump electromagnetic field with a center frequency at ωₚ and a Stokes electromagnetic field with a center frequency at ωₛ. The pump electromagnetic field and the Stokes electromagnetic field are then conveyed, in turn, through the dichroic mirror 106, through the coupling lens 110, the optical fiber 112 and the collimating lens set 114. The pump electromagnetic field and the Stokes electromagnetic field are then bounced off of the reflective surface of the 2D scanning mirror 116 and then conveyed through the objective lens set 118 onto a sample 120. The CARS signal, having center frequency ω_{AS}, reflected off of the sample 120 is then conveyed back through the objective lens set 118, reflected off of the reflective surface of the 2S scanning mirror 116, conveyed through, in turn, the collimating lens set 114, the optical fiber 112, and the coupling lens 110, reflected off of the reflective surface of the dichroic mirror 106, and conveyed through the optical filter 108 and processed by the PMT 110 to generate a signal for processing by the data acquisition system 122 to thereby determine the molecular composition of the sample 120.

In an exemplary embodiment, during the operation of the system 100, the control system 120 controls and monitors the operation of the 2D scanning mirror 116 such that the signals transmitted by the object lens set 118, namely the pump electromagnetic field with the center frequency at ωₚ and the Stokes electromagnetic field with a center frequency at ωₛ, scan the surface of the sample 120 using, for example, a raster scan pattern. In an exemplary embodiment, the optical fiber 112 is provided as an assembly that includes the collimating lens set 114, the 2D scanning mirror 116 and the objective lens set 118. In this manner, the optical fiber 112, the collimating lens set 114, the 2D scanning mirror 116 and the objective lens set 118 as a separate and self contained assembly in the form of a fiber probe system 126. In an exemplary embodiment, the optical fiber 112 has a low transmission loss characteristic in a broad wavelength range that may, for example, include a pump electromagnetic field with an infrared center frequency, a Stokes electromagnetic field with an infrared center frequency, and a CARS signal within the visible spectrum. In an exemplary embodiment, the optical fiber may be a signal mode fiber (“SMF”) for conveying the pump electromagnetic field and the Stokes electromagnetic field and may be a SMF or multimode fiber (“MMF”) for collection and conveyances of the CARS signal. In an exemplary embodiment, the optical fiber may be a MMF for collection and conveyances of the CARS signal in order maximize the efficiency of the collection of the CARS signal. In an exemplary embodiment, the 2D scanning mirror 118 may, for example, be implemented using a piezoelectric and/or a micro-electro-mechanical system (“MEMS”). In an exemplary embodiment, the fiber probe system 126 may be packaged within a hypodermic needle.

In an exemplary embodiment, an optical isolator may be operably coupled between the collimating lens set 114 and the reflective surface of the 2D scanning mirror 116.

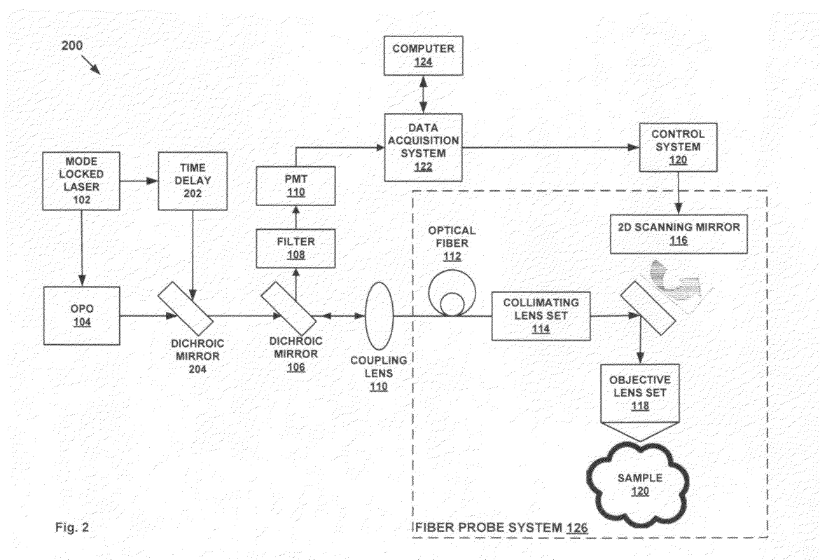

Referring now to FIG. 2, an exemplary embodiment of a micro-endoscopic system 200 is substantially identical in design and operation to the system 100 except as noted below. In an exemplary embodiment, the system 200 includes a conventional optical time delay 202 that is coupled to the output of the mode locked laser 122 for providing a time delayed signal to a reflective surface of a dichroic mirror 204 that is operably coupled to the OP 104 and a reflective surface of the dichroic mirror 106.

In an exemplary embodiment, during the operation of the system 200 an output signal from the mode locked laser 122 is time delayed by operation of the time delay 202. The time delayed output signal from the time delay 202 is then combined with the output signals of the OPO 104 by operation of the dichroic mirror 204. In an exemplary embodiment, during the operation of the system 200, the mode locked laser 102 generates the Stokes electromagnetic field and the OPO 104 generates the pump electromagnetic field. In an exemplary embodiment, during the operation of the system 200, the time delay 202 delays the Stokes electromagnetic field and then the and the dichroic mirror 204 combines the delayed Stokes electromagnetic field with the pump electromagnetic field such that the signals overlap with one another in space and time. The design and operation of the system 200 is otherwise substantially identical to the design and operation of the system 100.

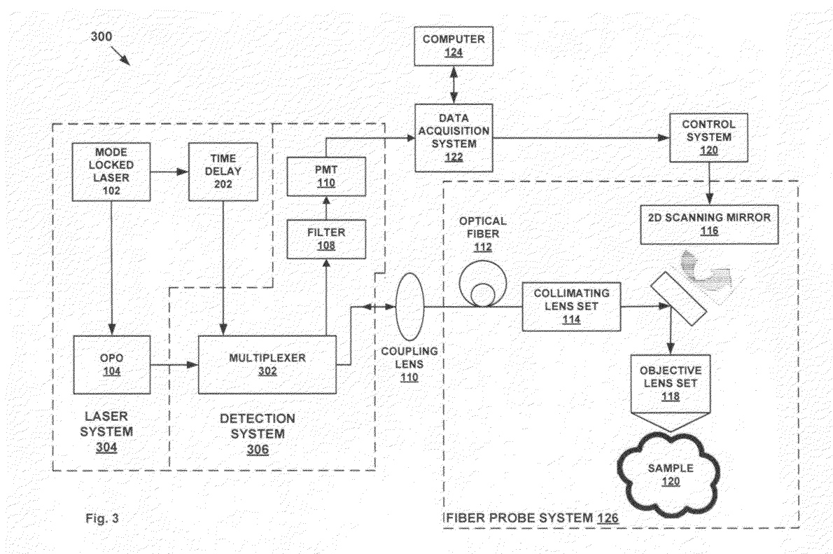

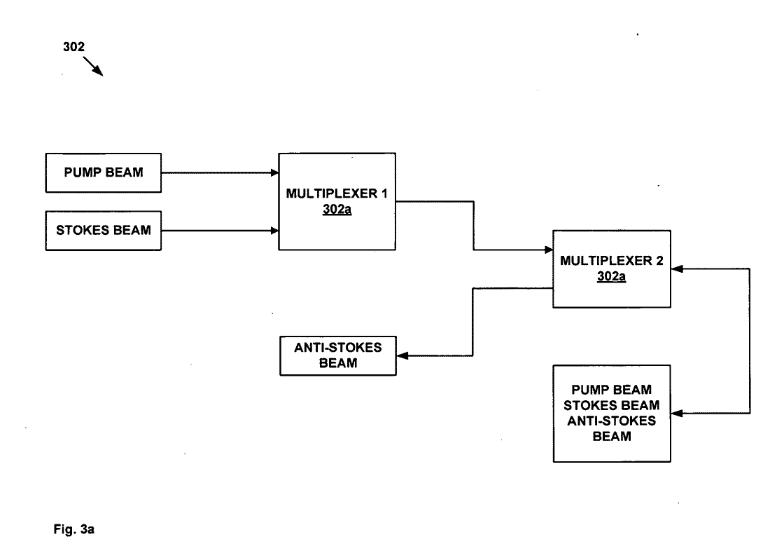

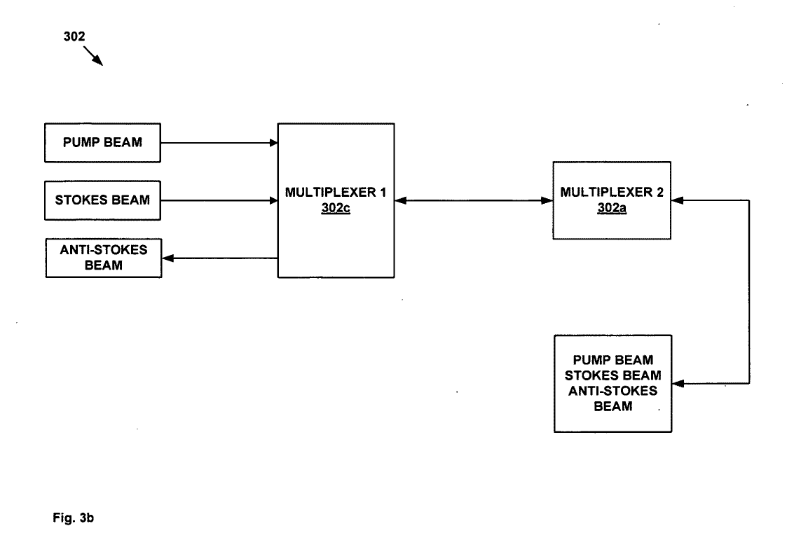

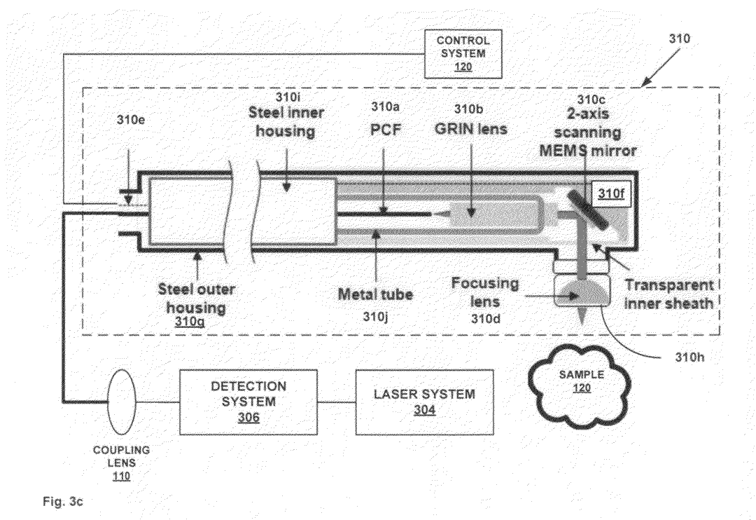

Referring now to FIG. 3, an exemplary embodiment of a micro-endoscopic system 300 is substantially identical in design and operation to the system 200 except as noted below. In an exemplary embodiment, the system 300 includes a wavelength division multiplexer 302 in place of the dichroic mirrors, 106 and 204. In this manner, electromagnetic signals may be routed during operation of the system 300.

In an exemplary embodiment, as illustrated in FIG. 3a, the wavelength division multiplexer 302 may include a plurality of multiplexers, 302a and 302b, that are cascaded to one another.

In an exemplary embodiment, as illustrated in FIG. 3b, the wave division multiplexer 302 may include a plurality of multiplexers, 302a and 302c, that are cascaded to one another.

In this manner, the system 300 provides a laser system 304 that includes the mode locked laser 102, the OPO 104 and the time delay 202. In this manner, the system 300 provides a detection system 306 that includes the filter 108, the PMT 110 and the multiplexer 302. The design and operation of the system 300 is otherwise substantially identical to the design and operation of the system 200.

Referring to FIG. 3c, in an exemplary embodiment, the system 300 includes a fiber probe system 310 that includes an optical fiber 310a that is operably coupled, at one end, to the coupling lens 110 and operably coupled, at another end, to an end of a gradient index (“GRIN”) collimating lens 310b. Another end of the GRIN lens 310b is operably coupled to a reflective surface of a 2-axis scanning MEMS mirror 310c. The reflective surface of the MEMS mirror 310c is further operably coupled to a focusing lens 310d oriented at a 90 degree angle relative to the axis of the GRIN lens 310b. The MEMS mirror 310c is further operably coupled to the control system 120 by one or more signal pathways 310e for controlling a 2-axis actuator 310f operably coupled to the reflective surface of the MEMS mirror. In an exemplary embodiment, the actuator 310f displaces the mirror 310c such that the mirror scans a 2-D region. In an exemplary embodiment, the actuator 310f displaces the mirror 310c such that the mirror provides a raster scan.

In an exemplary embodiment, the fiber probe system 310 is contained within an outer tubular housing 310g that defines an opening at one end for the fiber 310a and signal pathways 310e and an opening at another end that is transverse to the axis of the housing for permitting electromagnetic energy reflected off of the reflective surface of the MEMS mirror 310c to pass out of and into the housing. An end of a tubular support 310h is coupled to the other transverse opening of the housing 310g and another end of the tubular support houses and supports the focusing lens 310d.

In an exemplary embodiment, the fiber probe system 310 further includes an inner housing 310i and a further inner tubular support 310j for providing support to the fiber 310a and the GRIN lens 310b.

In an exemplary embodiment, the optical fiber 112 and/or the fiber 310a comprise a double clad photonic crystal fiber having: 1) single-mode operation for the excitation laser beams (i.e., the pump beam, the signal beam and the stokes beams); and 2) multimode operation for the collected signal (i.e., the CARS signal). In several experimental embodiments, we demonstrated that: a) the multimode operation for the collected signal was highly efficient; and b) low nonlinearity provided low signal distortion induced by the fiber. These were unexpected results.

In an exemplary embodiment, the system 300 may include a side-view probe and/or a front view probe configuration.

In an exemplary embodiment, one or more of the systems 100, 200 and 300 may be operated to identify cancer, and other, cells in patients in a multimodality image-guided intervention for cancer diagnosis using a computer tomography (“CT”) scan or magnetic resonance imaging (“MRI”) guided system to target a tumor. In an exemplary embodiment, one or more of the systems 100, 200 and 300 may then be operated to provide microendoscopy by inserting the fiber probe system 126 within the same cannula. With real-time optical imaging, the operator can directly determine the malignance of the tumor or perform fine needle aspiration biopsy for further diagnosis. During this operation, stable microendoscopy image series are needed to quantify the tissue properties, but they are often affected by respiratory and heart systole motion even when the interventional probe is held steadily. Thus, the present exemplary embodiments provide a microendoscopy motion correction (“MMC”) method using normalized mutual information (“NMI”)—based registration and a nonlinear system to model the longitudinal global transformations. Furthermore, in an exemplary embodiment, the MMC method includes the use of a Cubature Kalman filter to solve the underlying longitudinal transformations, which yields more stable and robust motion estimation. After global motion correction, longitudinal deformations among the image sequences are then calculated to further refine the local tissue motion. In addition, the exemplary embodiments of the MMC method may be used in any microendoscopy image processing system to correct for microendoscopy motion and/or in any image processing system to correct for motion. Furthermore, the exemplary embodiments of the MMC method may used in any image processing system to correct for motion. Finally, exemplary experimental results showed that compared to global and deformable image registrations, MMC method yields more accurate alignment results for both simulated and real data than convention motion correction methods.

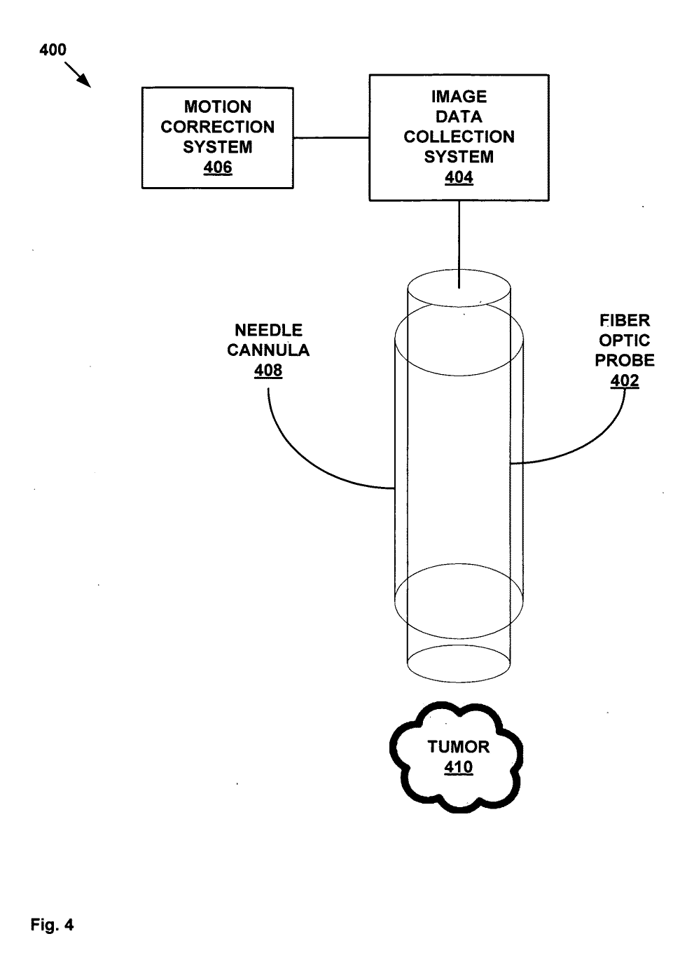

Referring now to FIG. 4, a minimally invasive multimodality image-guided (MIMIG) system 400 includes a fiber optic probe 402 that is operably coupled to an image data collection system 404. The image data collection system 404 is also operably coupled to a motion correction system 406 for implementing the MMC.

In an exemplary embodiment, the fiber optic probe 402 may be a conventional fiber optic probe that may, for example, be adapted to pass through a needle cannula 408 that is positioned proximate a tumor 410. In an exemplary embodiment, the fiber optic probe 402 may also include, or substitute, one or more aspects of the fiber probe system 126 of the systems 100, 200 and 300.

In an exemplary embodiment, the image data collection system 400 may be a conventional image data collection system. In an exemplary embodiment, the image data collection system 400 may also include, or substitute, one or more aspects of the systems 100, 200 and 300.

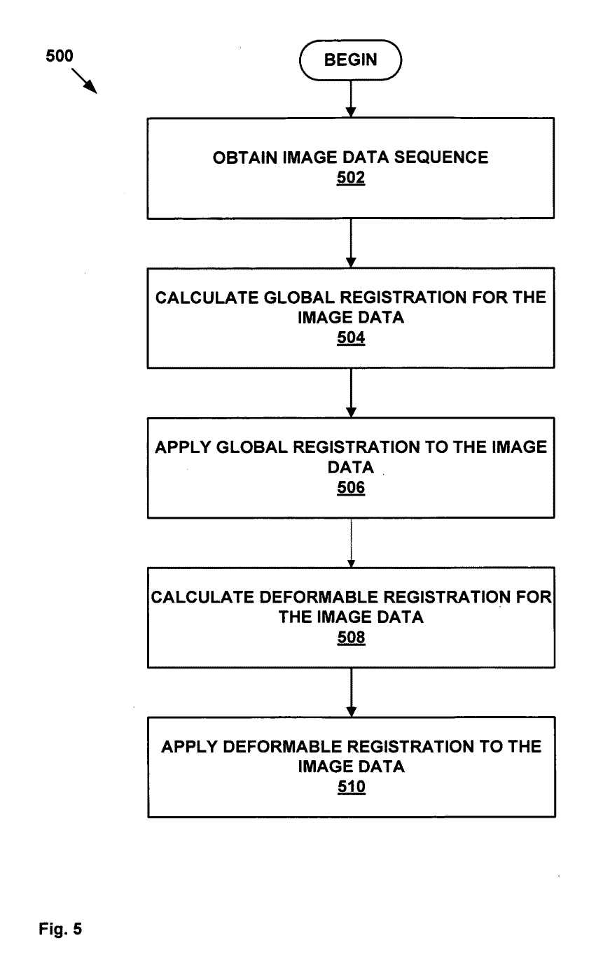

In an exemplary embodiment, as illustrated in FIG. 5, the motion correction system 406 implements a MMC method 500 in which; in 502, in 502, the motion correction system obtains an image sequence I={I₁, I₂, . . . , I_{N}}, where N is the number of image frames, from the image data collection system 404. In 504, the motion correction system 406 then calculates the global registration for the image data. In an exemplary embodiment, the global registration for the image data may be calculated in 504 in a conventional manner by, for example, calculating the global longitudinal transformations by maximizing the normalized mutual information. In 506, the motion correction system 406 then applies the global registration to the image data. In 508, the motion correction system 406 then calculates the deformable registration for the image data. In an exemplary embodiment, the deformable registration for the image data may be calculated in 508 in a conventional manner. In 510, the motion correction system 406 then applies the deformable registration to the image data.

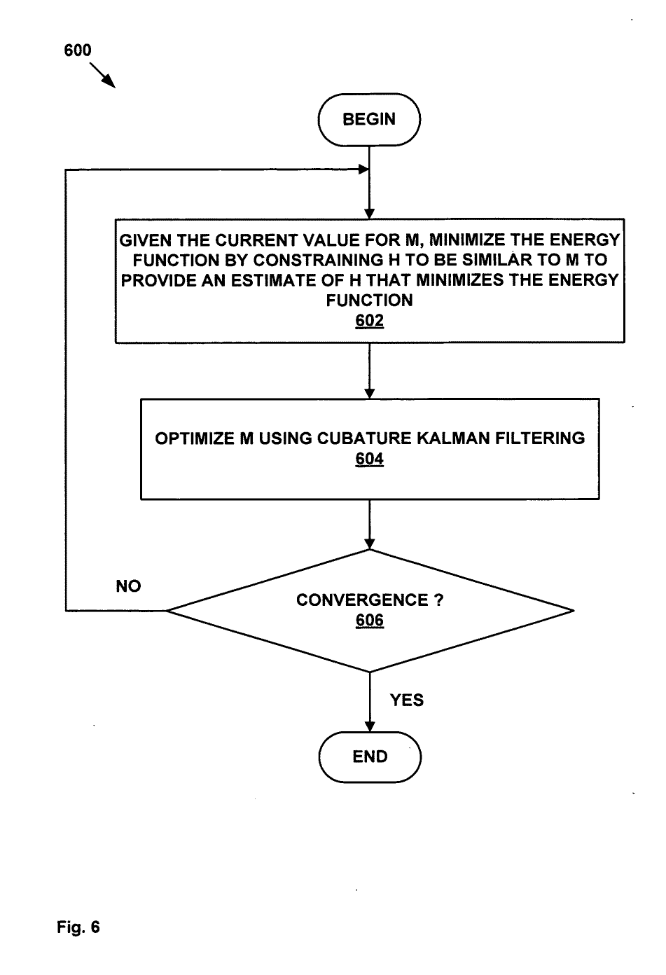

Referring now to FIG. 6, in an exemplary embodiment, in 504, the motion correction system 406 implements a method 600 for calculating the global registration for the image data in which the motion correction system iteratively minimizes an energy function E_{g}(M) to determine the value of M corresponding to minimum energy of the energy function, where M corresponds to the actual transformation, or serial alignment parameters, to be estimated:

$${E}_{g} ( M ) = \frac{1}{N - 1} {\sum }_{t = 1}^{N - 1} [ - NMI ( {H}_{t} \cdot {I}_{t} , {I}_{t + 1} ) + α {‖ {H}_{t} - f ( {M}_{t} ) ‖}^{2} ] , \qquad ( 1 )$$

where:

NMI refers to the normalized mutual information;

H={H₁, H₂, . . . , H_{N-1}} denotes the transformations, which may include either or both rigid and affine transformations, and may consist of serial translation, rotation, and scaling on actual images;

Hₜ∘ₜ, is the globally transformed image Iₜ onto image Iₜ₊₁;

α is a weighting factor;

Mₜ is the actual transformation;

uₜ is the system input;

$$\{ \begin{matrix}{M}_{t} = f ( {M}_{t - 1} , {u}_{t} ) + {n}_{t} \\ {H}_{t} = g ( {M}_{t} ) + {w}_{t} ,\end{matrix} \qquad ( 2 )$$

Mₜ is the actual transformation or state of the system;

ƒ(•) is the nonlinear system function and does not need an explicit form when a Cubature Kalman filter (“CKF”) is used;

uₜ is the system input;

nₜ and wₜ are independent;

g(•) is the system output function and is assumed to be an identity transformation; and

uₜ is the input signal and is assumed to be zero.

The first term in equation (1) ensures that the NMI between consequence images are maximized while the second term in equation (1) provides that the longitudinal transformations are subject to a nonlinear model. H is the observation or output of the nonlinear system and M is the actual transformation or system states. The method 600 estimates M, given the images sequences I and the measurable transformations H.

In particular, in 602, given a current value for M, the energy function E_{g}(M) is minimized, by constraining H to be similar to M, by the motion correction system 406 to provide an estimate of H that minimizes the energy function E_{g}(M). In 604, M is optimized by the motion correction system 406 by applying Cubature Kalman Filtering (“CKF”) to H. In 606, the motion correction system 406 determines if the numerical solution for estimating M is converging. If the numerical solution for estimating M is converging, then the method ends and the resulting value for M, which will typically be a matrix transformation, is used in 506 of the method 500.

In an alternative embodiment, in the method 600, augmented Kalman filtering (“AUKF”) may be used in addition to, or instead of, CKF in 604.

In an exemplary embodiment, in 508, the motion correction system 406 calculates the deformable registration for the image data by calculating the transformation vector v, from the image at timepoint t to the next timepoint t+1 by minimizing the following energy function E_{d}(v):

$${E}_{d} ( v ) = \frac{1}{N - 1} {\sum }_{t = 1}^{N - 1} {\int }_{Ω} {‖ {I}_{t + 1} [ {H}_{t} ( x ) + {v}_{t} ( x ) ] - {I}_{t} ( x ) ‖}^{2} + β {‖ ∇ {v}_{t} ( x ) ‖}^{2} , \qquad ( 3 )$$

where β is the weight of the smoothness constraint.

In an exemplary embodiment, a fast 2-D implementation of the deformable registration may be used. In an exemplary experimental embodiment, it was found that NMI is more robust for global registration since NMI reflects more global image features. In an exemplary embodiment, further local refinement using deformable registration may be provided using image intensity information since 1) image intensity reflects relatively local image features, and 2) the deformable registration refinement is constrained by the result of global registration and the smoothness constraint. As a result, both accuracy and robustness can be obtained.

In several exemplary experimental embodiments, the performance of the method 600 was evaluated. In a first exemplary experimental embodiment of the method 600, the dataset consisted of microendoscopy image sequences generated by applying simulated serial translation, rotation, and scaling on actual images captured during an image-guided intervention procedure using a Cellvizio 660 system with added Gaussian noise to the images. In a second exemplary experimental embodiment of the method 600, the dataset consisted of microendoscopy image sequences acquired in an image-guided intervention on a lung cancer rabbit model.

In the first exemplary experimental embodiment of the method 600, ten simulated microendoscopy sequences, using real microendoscopy frames collected from the rabbit experiments, were used. The longitudinal transformations were simulated using sine and cosine signals,

pᵢ(t)=aᵢ sin(bᵢt+φᵢ)+cᵢ, (4)

where t=1, . . . , N−1, i=1, . . . , 4;

aᵢ, bᵢ, and cᵢ are the amplitude, frequency and shifting of the transformation signals, respectively; and

p₁, p₂, p₃, p₄ represent the translations in x- and y-directions, rotation, and scaling.

In the first exemplary experimental embodiment of the method 600, the typical amplitude for translation was set to 10 pixels, rotation angles were [−20, +20] degrees, and frequency was between 0.5 and 1. The scaling range was between 0.98 and 1.02.

In the first exemplary experimental embodiment of the method 600, image sequences were generated by first transferring the reference images using the simulated transformations and then adding spatially correlated Gaussian noises. Because only global longitudinal transformations were simulated, we compared the results of the method 600 with conventional NMI-based motion correction for global registration.

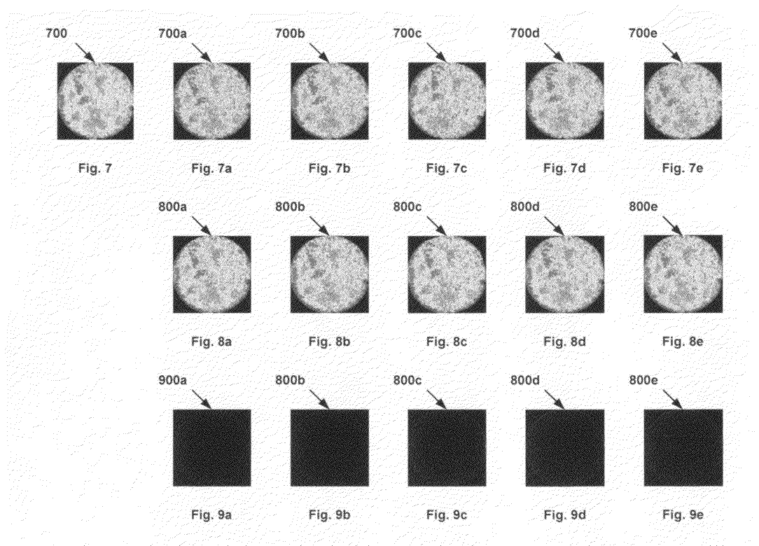

As illustrated in FIGS. 7 and 7a-7e, in the first exemplary experimental embodiment of the method 600, a reference image 700 was used to generate a series of simulated images, 700a-700e. As illustrated in FIGS. 8a-8e, the method 600 was then implemented to correct for motion in the simulated images, 700a-700e, to thereby generate motion corrected images, 800a-800e.

In order to characterize the results of the first exemplary experimental embodiment of the method 600, as illustrated in FIGS. 9a-9e, the difference, 900a-900e, between the reference image 700 and each of the motion corrected images, 800a-800e, respectively, was generated. As demonstrated by the difference images, 900a-900e, the difference between the reference image 700 and the motion corrected images, 800a-800e, is virtually zero. This was an unexpected result.

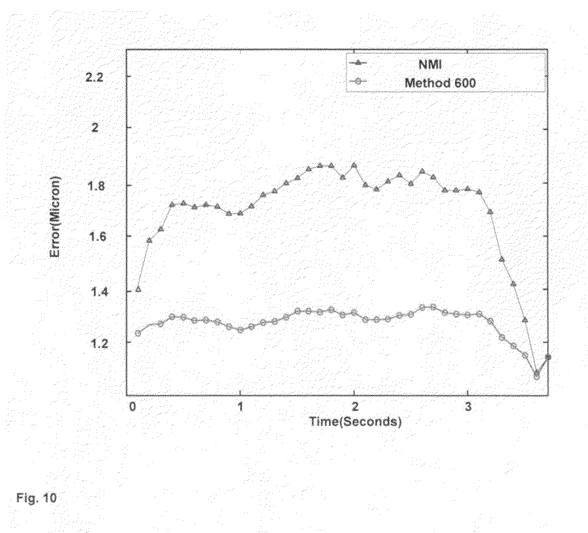

Furthermore, in order to further characterize the results of the first exemplary experimental embodiment of the method 600, as illustrated in FIG. 10, the results of the first exemplary experimental embodiment of the method 600 were compared with an embodiment of a conventional NMI-based method of motion correction for global registration applied to simulated images. In particular, the amount of error between the results of the first exemplary experimental embodiment of the method 600 and the reference image 700 and the amount of error between the results of the conventional NMI-based method and the reference image 700 were compared. As illustrated in FIG. 10, the amount of error was significantly less for the first exemplary embodiment of the method 600 versus the conventional NMI-based method of motion correction for global registration. This was an unexpected result.

Furthermore, in order to further characterize the results of the first exemplary experimental embodiment of the method 600, as illustrated in FIG. 10, the results of the first exemplary experimental embodiment of the method 600 were compared with an embodiment of a conventional NMI-based method of motion correction for global registration applied to the simulated images. In particular, as illustrated below in Table 1, the average errors of the longitudinal transformations between the reference image 700 and the alignment results obtained using the method 600 and the conventional NMI method were determined and demonstrated that the method 600 provided significantly better results.

TABLE 1
The average errors and standard deviation for the ten
simulated image sequence (units in micron).

Motion Correction Method

| | NMI-based global | |
|---|---|---|
| | registration | Method 600 |
| Average | 2.2 | 1.3 |
| Error | | |
| (microns) | | |
| Standard | 0.3 | 0.1 |
| Deviation | | |

Furthermore, the comparative results in Table 1 above demonstrated that the method 600 yields a significantly more accurate estimation of the longitudinal transformations in motion correction of image sequences. The exemplary comparative experimental results above demonstrated that the method 600 provides more accurate motion correction than that provided by conventional motion correction methods such as NMI-based motion correction for global registration. A conventional paired t-test of the exemplary experimental results detailed above also showed that such accuracy improvement is statistically significant (p<0.05). The improved results provided by the method 600 in the first exemplary experimental embodiment detailed above were unexpected.

In the second exemplary experimental embodiment of the method 600, microendoscopy videos were collected from lung cancer rabbit model experiments during image-guided intervention. After cutting the microendoscopy videos into different clips based on image similarities, the method 600, the conventional NMI-based motion correction method for global registration, and the conventional NMI motion correction method for global registration plus a conventional deformable registration were applied to 30 microendoscopy video clips. For each video clip, an image with the smallest difference to its neighboring frames was selected as the reference image, and all the other images were aligned with this reference image. The results obtained from the application of the method 600, the conventional NMI-based motion correction method, and the conventional NMI-based motion correction method plus a conventional deformable registration to the 30 microendoscopy video clips were them compared using a conventional NMI metric as a proxy for accuracy in the global registration provided by the respective motion correction methods. As illustrated below in Table 2, the method 600 provided significantly improved results versus both of the conventional motion correction methods.

TABLE 2
The average NMI values in each video.

| | Motion Correction Method | | | |
|---|---|---|---|---|
| | | (b) MNI-based | | |
| | (a) NMI- | global | | |
| | based | registration + | (c) | Improvement |
| | global | deformable | Method | between |
| | registration | registration | 600 | (b) and (c) (%) |
| Average | 0.58 | 0.63 | 0.70 | 12.7% |
| NMI | | | | |
| Standard | 0.07 | 0.08 | 0.08 | |
| Deviation | | | | |

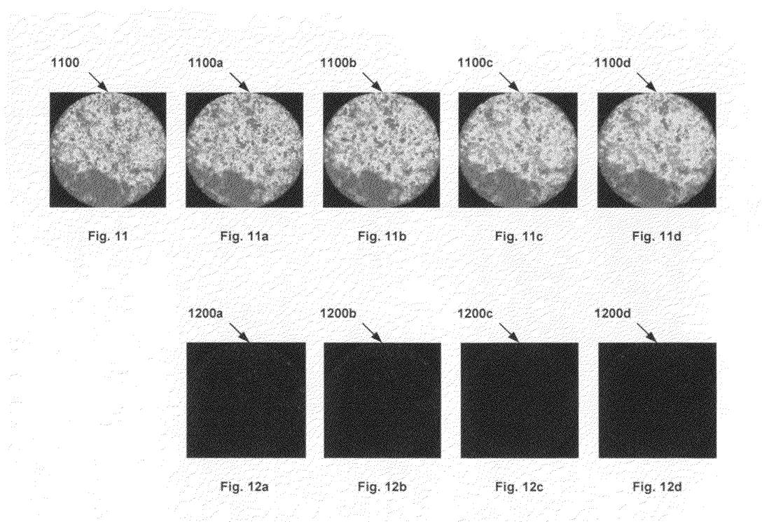

In a third exemplary experimental embodiment of the method 600, referring now to FIGS. 11, 11a-11d and 12a-12d, for further validation of the performance of the method, 30 microendoscopy video clips were collected from eight rabbit experiments. After cutting the whole microendoscopy videos into different video clips, an image with the least difference to its neighboring images was selected as the reference frame 1100 for each video clip, and a current frame 1100a was then aligned with this reference frame using the method 600 and the conventional NMI-based motion correction method for global registration. The third exemplary experimental embodiment of the method 600 provided a motion correction frame 1100b provided using the conventional NMI-based global registration method and motion corrected images, 1100c and 1100d, for the method 600. Note that motion corrected image 1100c was generated by the method 600 after one iteration and the motion corrected image 1100d was generated by the method 600 after two iterations.

In the third exemplary experimental embodiment of the method 600, in order to further validate and compare the results of the method 600 with the results for the conventional NMI-based motion correction global registration method, difference images, 1200a, 1200b, 1200c and 1200d, were generated which illustrate: the difference between the image 1100a and the reference image 1100, the difference between the image 1100b obtained using the conventional NMI-based motion correction global registration method and the reference image 1100, the difference between the image 1100c obtained using the method 600 and the reference image 1100, and the difference between the image 1100d and the reference image 1100. A visual examination of the difference images clearly indicates that the method 600 was far superior to the conventional NMI-based motion correction global registration method. Furthermore, as a further validation of the exemplary experimental results, conventional NMI metrics were also calculated for the motion corrected images obtained using the method 600 and the conventional NMI-based motion correction global registration method. The results of these NMI metric calculations indicated that the accuracy of image alignment was better for the method 600 versus the NMI-based motion correction global registration method for all the 30 image sequences studied. The average improvement provided by the method 600, versus the conventional NMI-based motion correction global correction method, was 6.6% and the largest one being 12.46%. All of the improved results provided by the method 600 were unexpected results.

In an exemplary embodiment, in 504, the motion correction system 406 calculates the global registration for the image data using a conventional hidden Markov model (“HMM”) method for motion correction. Basically, the conventional HMM method assumes that the motion of the current line to the next line in a scanned image remains the same or is most likely not changing. A standard exponential model, or distribution, is typically used to describe this assumption. Essentially, this assumption is only realistic when the patient is still or relatively still, the relative position for the scanning beam and patient's region of interest stays constant, but this situation is not very common because a patient's motion may be disordered and random and may include sudden changes of speed all of the time during the procedure. Thus while HMM may work effectively for the resting stage, it might fail and give a wrong estimation during a rapid movement stage.

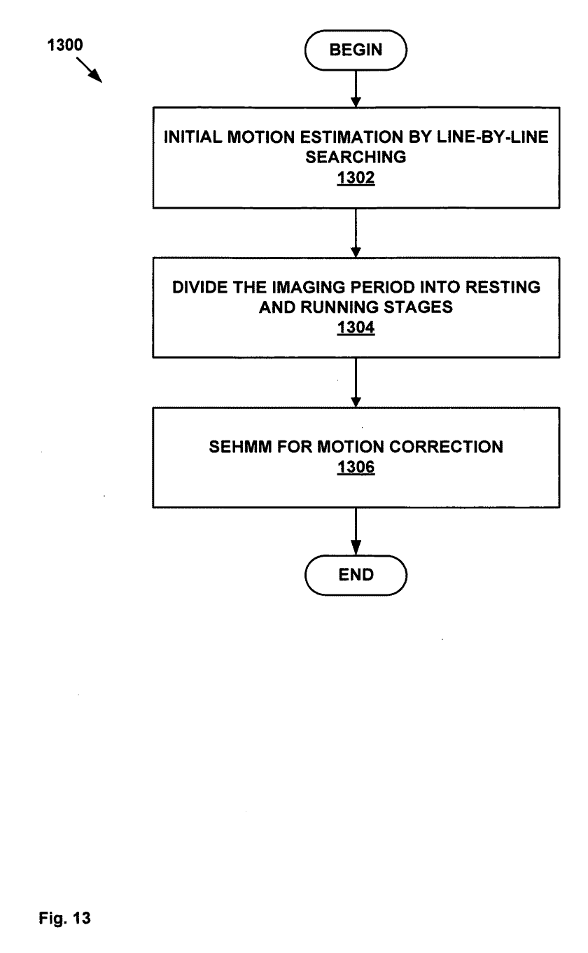

Thus, as illustrated in FIG. 13, in an exemplary embodiment, in 504, the motion correction system 406 calculates the global registration for the image data using an extended version of HMM by incorporating an estimated speed into the state transition model for more accurate estimation to provide a speed embedded HMM (“SEHMM”) method 1300. In particular, in 1302, an initial motion estimation is achieved by using a line-by-line searching method. Then, in 1304, a grouping algorithm is used to divide the whole imaging period into resting and running stages for speed estimation. Finally, in 1306, SEHMM is then adopted for motion correction.

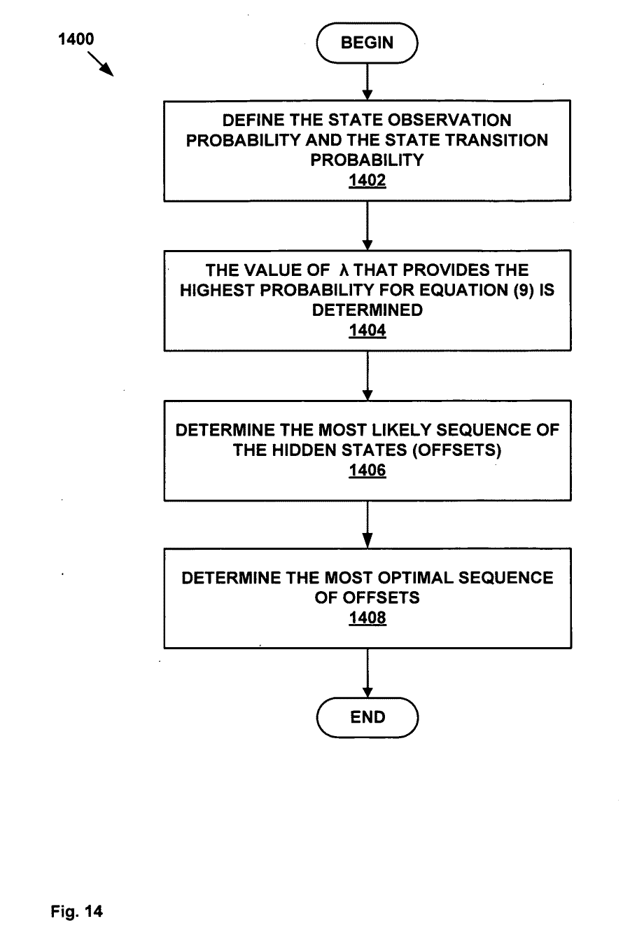

In an exemplary embodiment, as illustrated in FIG. 14, in 1306, the motion correction system 406 implements a SEHMM method 1400 for motion correction.

As described above, in general, the systems 100, 200 and 300 capture a series of images by passing a focus of laser excitation repeatedly over region of a sample 120 and collecting the resulting photons via a photon multiplier 110. Although the motion of the sample 120 is in 3-D, Z-axis motion shift is typically less than 1 μm for a scanning speed of 2 ms/line, much lower than the X (medial-lateral direction along the raster scan line) and Y (rostral-caudal direction across the raster scan line) directions. Therefore, the primary task of the method 1400 is to estimate the motion in X and Y directions. Due to the motion of the sample 120 during the raster scan progression, the relative motion can be written as,

$$\{ \begin{matrix}{X}_{i}^{′ k} = {X}_{i}^{k} + {δ}_{x}^{k} \\ {Y}_{i}^{′ k} = {Y}_{i}^{k} + {δ}_{y}^{k} ,\end{matrix} \qquad ( 5 )$$

where Xₜ^{k}(t)={t/(τ/N)}·N and Xₜ^{k}(t)=[t/(τ/N)] are the actual location of an object point;

(δₓ^{k},δ_{y}^{k}) is its offset due to motion;

N×N is the size of each frame;

[•] represents the integer operation;

{•} denotes the fractional operation;

τ is the scanning time for a frame.

In an exemplary embodiment, the laser output of the systems 100, 200 and 300 moves in a zigzag pattern in X-direction and a step function pattern in Y-direction. Thus, the goal of the method 1400 is to estimate the offsets (δₓ^{k},δ_{y}^{k}) from the serial images. Equation (5) assumes that each line has the same relative displacement, so we can choose a line-by-line motion correction algorithm to solve this problem. The reason is that the shifts for all the pixels within a line do not get beyond one-pixel in the Y-direction and are very tiny in X direction. Although pixel-by-pixel correction can yield more accurate results, one has to trade off between speed and the gain in accuracy.

The SEHMM method 1400 is an extension of the conventional HMM method by using a motion prediction model with HMM to better estimate the state transition probability. In particular, denoting the displacement state for line k as (δₓ,δ_{y}), it is necessary to define the state observation probability πₖ^{δ}^{ₓ}^{,δ}^{_{y}} and the state transition probability T[(δₓ^{k-1},δ_{y}^{k-1})→(δₓ^{k},δ_{y}^{k})]. During the operation of the SEHMM method 1400, the transition probability of the displacement state is estimated based on the estimated moving speed. Thus, the SEHMM method 1400 can match motion more accurately not only during the resting stage but also during the moving stage.

The transition probability is defined as:

$$T ( {δ}_{x}^{k - 1} , {δ}_{y}^{k - 1} ) -> ( {δ}_{x}^{k} , {δ}_{y}^{k} ) = \frac{1}{2 πλ} {e}^{- r / λ} , \qquad ( 6 )$$

where r is defined as:

$$r = \sqrt{{( {δ}_{x}^{k} - ( {δ}_{x}^{k - 1} + {v}_{x}^{k - t , k} {τ}_{line} ) )}^{2} + {( {δ}_{y}^{k} - ( {δ}_{y}^{k - 1} + {v}_{y}^{k - t , k} {τ}_{line} ) )}^{2}} , \qquad ( 7 )$$

where νₓ^{k 1,k} and ν_{y}^{k-1,k} are the estimated speed of the motion from line k−1 to k for X- and Y-directions, respectively; and

τₗᵢₙₑ is the scanning time for a line.

By using Eq. (7), if the moving speed is estimated at line k−1, the offset at line k can be estimated. Therefore, in the SEHMM method 1400, we no longer assume that the state transition probability is the highest when the sample 120 does not move. On the contrary, this probability gets its peak at a linearly estimated offset value. Since the goal for motion correction is to estimate δₓ^{k},δ_{y}^{k} for each line k from a given reference frame R and the current image I, we implement it by maximizing a posteriori,

P=(δₓ^{k},δ_{y}^{k}|Iᵢ^{k},R)=P(Iᵢ^{k}|R(X′ᵢ^{k},Y′ᵢ^{k}))·P(δₓ^{k},δ_{y}^{k}|δₓ^{k-1},δ_{y}^{k-1})=πₖ^{δ}^{ₓ}^{,δ}^{_{y}}·T(δₓ^{k-1},δ_{y}^{k-1}→δₓ^{k},δ_{y}^{k}). (8)

Expressing P as a logarithm probability, one gets,

ln(P)=ln(πₖ^{δ}^{ₓ}^{,δ}^{_{y}})+ln(T). (9)

The first term in Eq. (9) is the state observation probability πₖ^{δ}^{ₓ}^{,δ}^{_{y}} and reflects the goodness of matching between a line in the current frame I and the corresponding line in the reference frame R. Denoting the intensity of the ith pixel in the kth line of the current frame as Iᵢ^{k}, and that of the corresponding pixel in the reference frame as (x′ᵢ^{k},y′ᵢ^{k}), the observation probability can be modeled as a discrete Poisson distribution explicitly, π_{k,i}^{δ}^{ₓ}^{,δ}^{_{y}}=(γR)^{γI}·e^{-γR}I(γI)!, where γ is the calibration factor of the photon number, which is an inherent factor in an imaging system. Taking the logarithm transformation of π_{k,i}^{δ}^{ₓ}^{,δ}^{_{y}} we get,

ln(π_{k,j}^{δ}ₓ^{,δ}^{_{y}})=γI ln(γ)+I ln(R)−ln((γI)!)−γ^{R} (10)

where γ and Iᵢ^{k} are independent of the changing offsets, and R is indeed a function of the offsets. Thus equation (10) can be simplified as,

ln(π_{k,i}^{δ}^{ₓ}^{,δ}^{_{y}})∝γ(I ln(R)−R). (11)

Then, the observation probabilities for all the pixels within line k can be calculated by,

$$ln ( {π}_{k}^{{δ}_{x} , {δ}_{y}} ) = {\sum }_{i = 1}^{N} ln ( {π}_{k , i}^{{δ}_{x} , {δ}_{y}} ) . \qquad ( 12 )$$

Thus, in an exemplary embodiment, the method 1400, in 1402, defines the state observation probability πₖ^{δ}^{ₓ}^{,δ}^{_{y}} and the state transition probability T[(δₓ^{k-1},δ_{y}^{k-1})→(δₓ^{k},δ_{y}^{k})].

Once the state observation probability and the state transition probability are defined in 1402, the best displacement signal can be calculated by maximizing Eq. (9), by following two iterative steps:

First, in 1404, the value of λ that leads to the highest probability for Eq. (9) is determined. In an exemplary embodiment, the value of λ that leads to the highest probability for Eq. (9) may be determined by systematically scanning values of λ in a prescribed range. In an exemplary embodiment, the values of λ may be scanned uniformly in log space to get constant percentage sampling. For each value of λ, the method 1400 may be implemented to find the most optimal offset sequence and calculate its total probability. In an exemplary embodiment, the value of λ chosen in 1402 is the one with the most probable offset sequence.

Then, in 1406, the most likely sequence of the hidden states, offsets, may be determined using a Viterbi algorithm. In an exemplary embodiment, this can be accomplished in two steps. First, determine the most probable offset sequence for every state at time, line, k from any of the states at time, line, k−1 by marching forward through the time domain. Then, in 1408, a backtrack along the path of the most probable offset sequence to record the results of optimal offset sequence. In an exemplary embodiment, a line-by-line search is first used to estimate the initial offset, and the speed of the motion can be estimated by applying smoothness filter temporally on the estimated offsets before implementing the SEHMM method 1400.

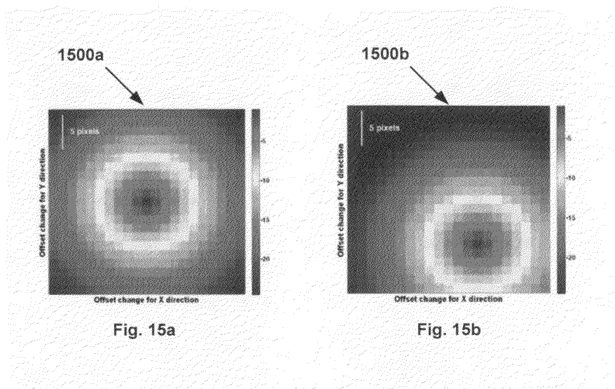

In order to illustrate how the SEHMM method 1400 works, referring now to FIGS. 15a and 15b, exemplary state transition probabilities for HMM and SEHMM models, 1500a and 1500b, respectively, are illustrated. The center of each distribution plot is set to (δₓ^{k-1},δ_{y}^{k-1}), and it can be seen that the peak of the state transition probability 1500a is right in the middle for the HMM model while the peak for the SEHMM state transition probability 1500b is shifted as a result of the motion estimation provided by operation of the method 1400. A shifting of the peak of the probability function indicates that a motion is assumed from one line to another. This compensation for adding the estimated speed value from the current offset to the following offset can remarkably improve the estimation accuracy because it satisfies the prediction requirement in all the time points but not only in the still time points.

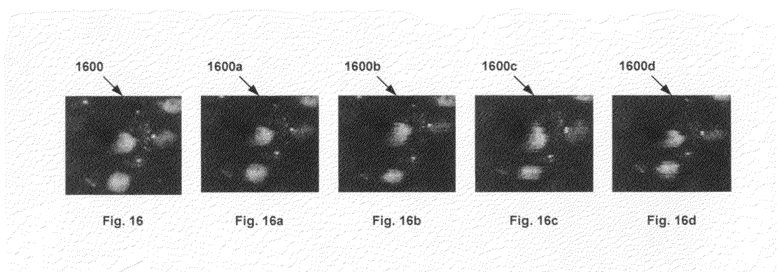

In an exemplary experimental embodiment of the SEHMM method 1400, in order to validate the method, as illustrated in FIGS. 16 and 16a-16d, simulated serial images were used to quantitatively validate the SEHMM method by comparing the SEHMM method with a conventional HMM method. Temporal displacements were first generated to transfer a real 2D image, 1600, to produce longitudinal image sequences, 1600a-1600d. The displacement signal, for generating the images, 1600a-1600d, was generated on a pixel-by-pixel basis using a first differential equation with a characteristic time constant driven by a combination of the random and deterministic processes to simulate the effect of acquiring laser-scanning data from a moving sample:

$$\{ \begin{matrix}{\vec{φ}}_{x}^{t + 1} = ( 1 - \frac{1}{τ} ) {\vec{φ}}_{x}^{t} + \frac{1}{τ} ( {\vec{ζ}}_{{σ}_{x}} + {\vec{D}}_{x}^{t} ) \\ {\vec{φ}}_{y}^{t + 1} = ( 1 - \frac{1}{τ} ) {\vec{φ}}_{y}^{t} + \frac{1}{τ} ( {\vec{ζ}}_{{σ}_{y}} + {\vec{D}}_{y}^{t} ) ,\end{matrix} \qquad ( 13 )$$

where \vec{φ}ₓ′ and \vec{φ}_{y}′ denote the simulated offsets for X- and Y-directions, respectively; and

t expresses the time-point;

t=(k−1)·N+i;

τ is set to 500;

σₓ and σ_{y} (σₓ=4, σ_{y}=40) are the standard deviations of the Gaussian random variables \vec{ζ}_{σ}_{ₓ} and \vec{ζ}_{σ}_{_{y}}; and

\vec{D}ₓ′ and \vec{D}_{y}′ are the step functions at the time t.

In the exemplary experimental embodiment of the SEHMM method 1400, Poisson noise was added on a pixel-by-pixel basis to simulate the photon counting statistic because the values during scanning are Poisson distributed in terms of photon numbers but not in units of pixel intensity. σₓ, σ_{y} and τ control the amplitudes of temporal dynamic displacements.

In the exemplary experimental embodiment of the SEHMM method 1400, two simulated data sets, T1 and T2, were generated with these default parameters. In addition, the values of σₓ, σ_{y} and τ were changed and the other parameters remained constant. Two more datasets, T3 and T4, were simulated by setting σₓ10, σ_{y}=30 and τ=200. The number of frames for T1, T2, T3, and T4 were 65, 82, 56 and 51, respectively. The image size was 129×129, and the resolution was 0.39 μm/pixel.

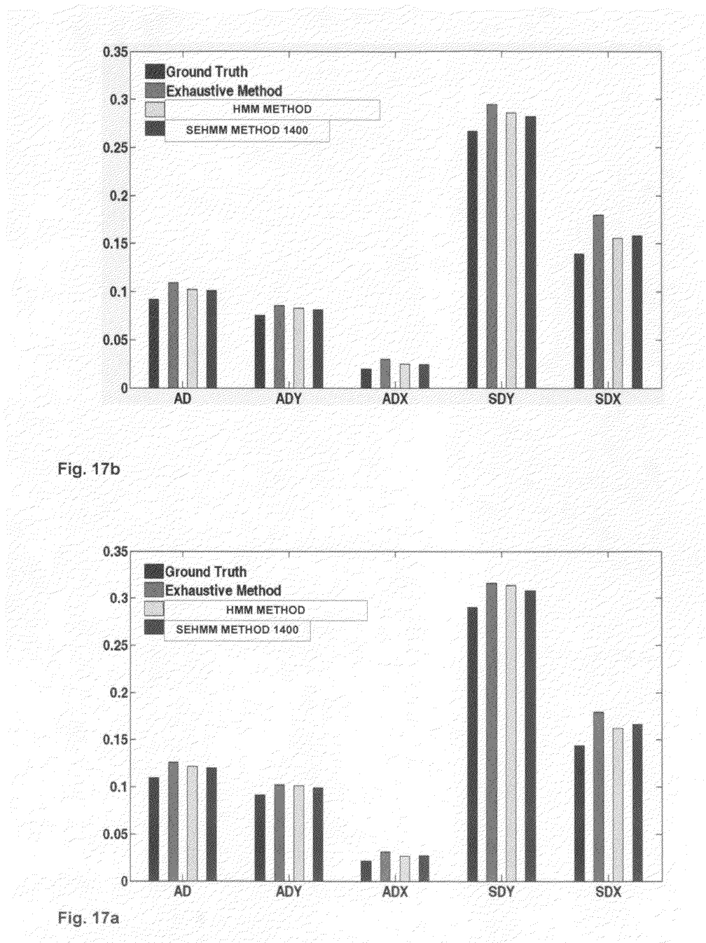

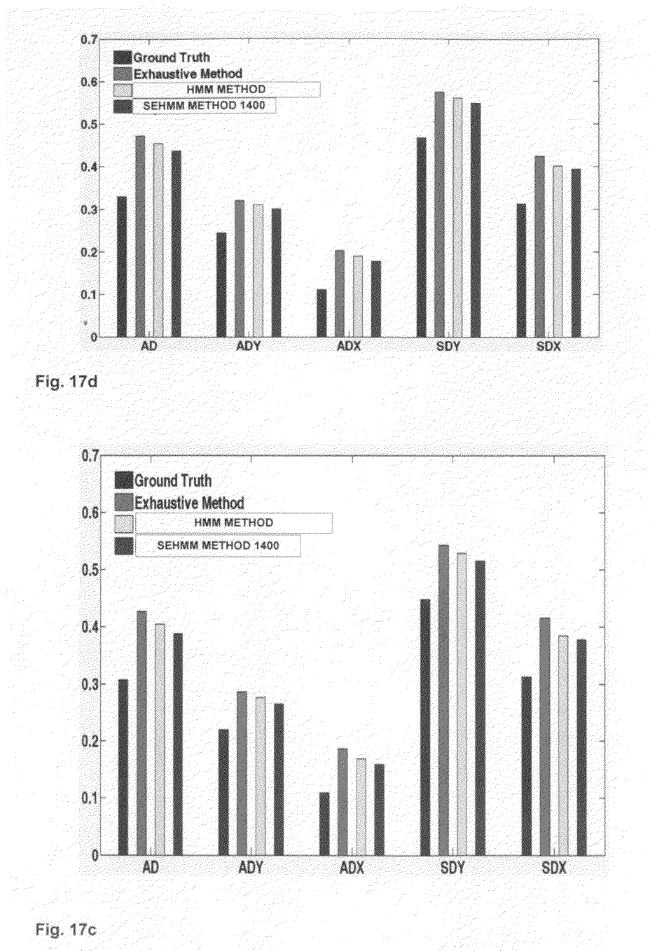

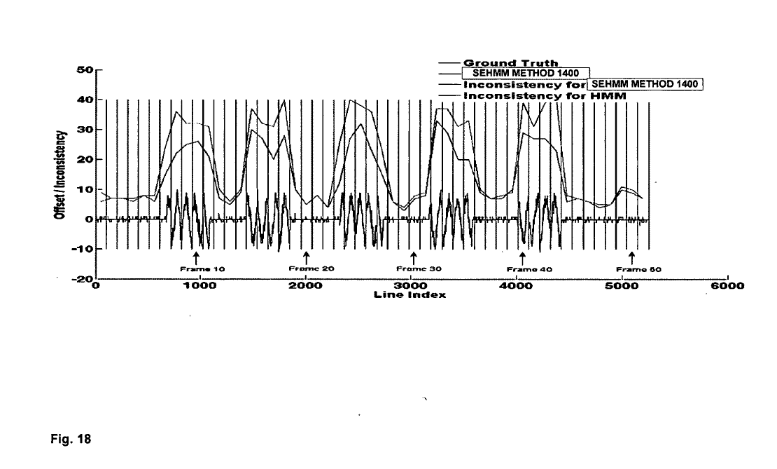

In the exemplary experimental embodiments of the SEHMM method 1400, the exemplary experimental results for the conventional HMM method were denoted as (δₓ^{k},δ_{y}^{k}), and the exemplary experimental results for the SEHMM method were denoted as (δ′ₓ^{k},δ′_{y}^{k}), the simulated offsets were denoted as (φₓ^{t},φ_{y}^{t}), and the exhaustive search results denoted as (\hat{δ}ₓ^{k},\hat{δ}_{y}^{k}), and we compared the motion correction accuracy provided by the different methods. Because all of methods tested were line-by-line motion correction methods and the ground truth was stored in pixel resolution, we took the mean value of all ground truth offsets within each line and then rounded the mean value to the nearest integral, i.e., φₓ^{t}φ_{y}^{t}→φₓ^{k},φ_{y}^{k}. The difference between these two were also compared, which indicated the accuracy of the line-by-line based methods. We used the following five performance measures: average distance (“AD”), average distance for Y-direction (“ADY”), average distance for X-direction (“ADX”), standard deviation for Y-direction (“SDY”) and standard deviation for X-direction (“SDX”), to evaluate the performance of the different methods. FIGS. 17a-17d illustrate the comparison results between the four line-by-line results and the ground truth for data sets, T1, T2, T3, and T4, respectively. The dark blue bars show differences between the rounded line-by-line ground truth and the simulated pixel-by-pixel ground truth, and other bars show the results of the three methods, namely the exhaustive method, the HMM method, and the SEHMM method 1400. It can be seen that for the performance measures used, the SEHMM method 1400 yielded the smallest errors (some with slightly larger SDX).

In the exemplary experimental embodiments of the SEHMM method 1400, we also focused on the Y-direction because the offsets in this direction have relatively large movement than in X-direction. FIG. 18 illustrates the results for the simulated dataset T4. In FIG. 18, the line-by-line rounded ground truth, the results of the SEHMM method 1400, and the results of the conventional HMM method are shown. Furthermore, the inconsistency between the results of the SEHMM method 1400, and the results of the conventional HMM method are also shown. The inconsistency was calculated as the number of lines within a frame for which the results of SEHMM method 1400 and the conventional HMM method were different from the ground truth. As illustrated in FIG. 18, during resting stage, such inconsistency is small, and both the SEHMM method 1400 and the conventional HMM method yielded good results, about 10 lines were different, but during the movement stage, the SEHMM method 1400 reduces the inconsistency as compared with the results of the conventional HMM method. All of these exemplary experimental results were unexpected.

In an another exemplary experimental embodiment of the SEHMM method 1400, the mean absolute intensity difference was used as the measure to show the performance of the methods. The mean absolute intensity difference across longitudinally corresponding pixels reflects the goodness of matching for the image sequences. Table 3 below shows the comparative results for datasets, T5 and T6, provided by an anonymized group, respectively, using the different methods. The number of frames was 1200 and 200, image size was 128×128 and 256×256, respectively, and the resolution was 0.39 μm/pixel for both. The improvement can be seen from the comparison results when we adopt the proposed method.

TABLE 3
Average of absolute intensity differences for real datasets T5 and T6

| Data | Original | Exhaustive | | |
|---|---|---|---|---|
| Set | Data | Method | HMM | SEHMM |
| T5 | 7.05 | 5.89 | 5.48 | 5.03 |
| T6 | 9.54 | 7.74 | 7.15 | 6.56 |

In the other exemplary experimental embodiment of the SEHMM method 1400, the SEHMM method 1400 again provided superior accuracy versus the conventional HMM method. This was an unexpected result.

Thus, the exemplary experimental embodiments of the SEHMM method 1400 demonstrated that a SEHMM method for motion correction provided far superior accuracy to conventional motion correction methods for global registration such as HMM. In particular, the SEHMM method 1400 provided a much better estimate of the state transition probability compared to the conventional HMM method that assumed no motion always has the highest probability. Furthermore, the SEHMM method 1400 can model the motion more accurately and operates directly on the motion-distorted image data without any external signal measurement such as the sample movements, heartbeat, respiration, or muscular tension. Using simulated and real images, it was demonstrated that the SEHMM method 1400 was more accurate than the conventional HMM method—using both simulated and real image sequences. This was an unexpected result.

Thus, in the exemplary experimental embodiments of the SEHMM method 1400, a quantitative validation was performed to compare conventional HMM with the SEHMM method 1400 based on both simulated data and real data. First, simulated image sequences were generated to mimic various real motion situations, and different dynamic amplitudes were applied to make the validation more realistic and reasonable. For real data, the comparative results demonstrated the performance of the SEHMM method 1400. The exemplary experimental embodiments results showed that the SEHMM method 1400 achieved higher estimation accuracy and image alignment results as compared with conventional HMM, especially in the running stages of the image sequences.

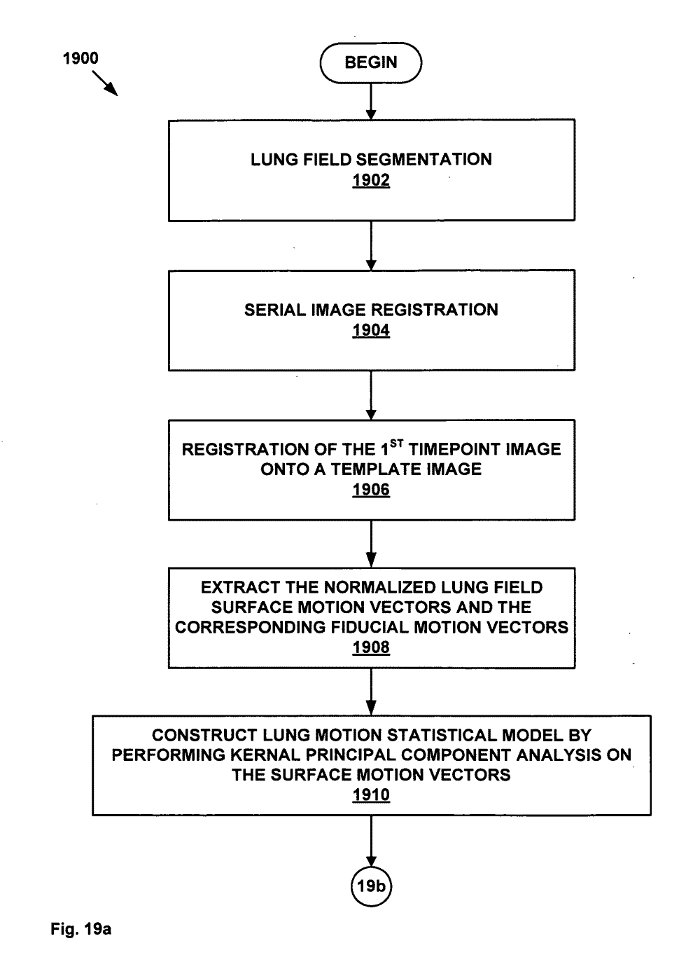

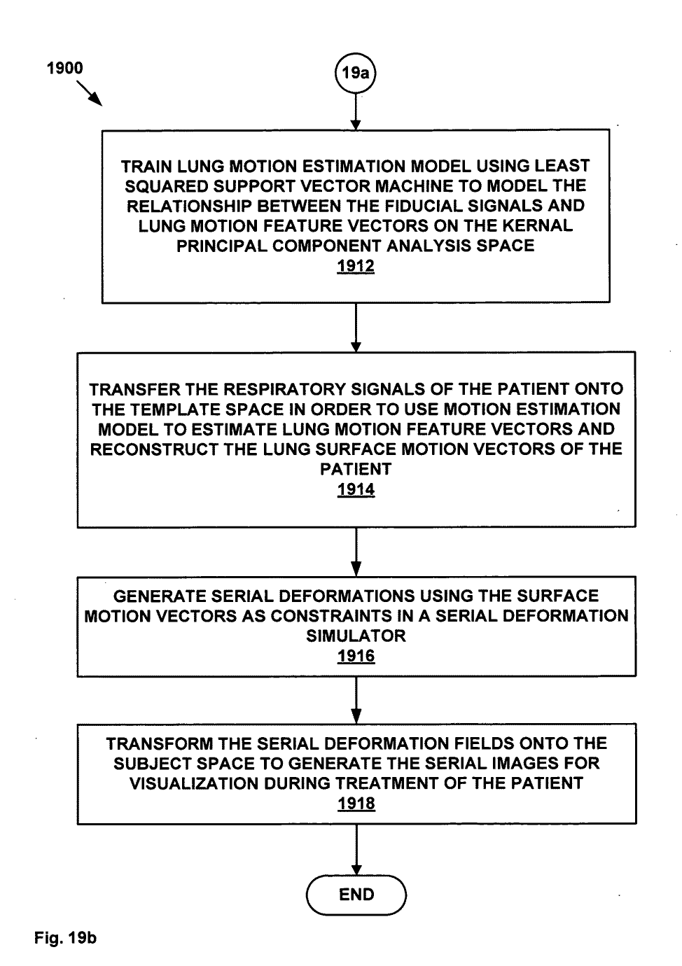

In an exemplary embodiment, as illustrated in FIGS. 19a and 19b, the motion correction system 406 may be used, for example, to correct for breathing motion in images obtained from lung tissue, by implementing a method 1900 for motion correction. More generally, the teachings of the method 1900 may be used to correct for motion in any type of sample 120 to provide global registration of a sequence of images.

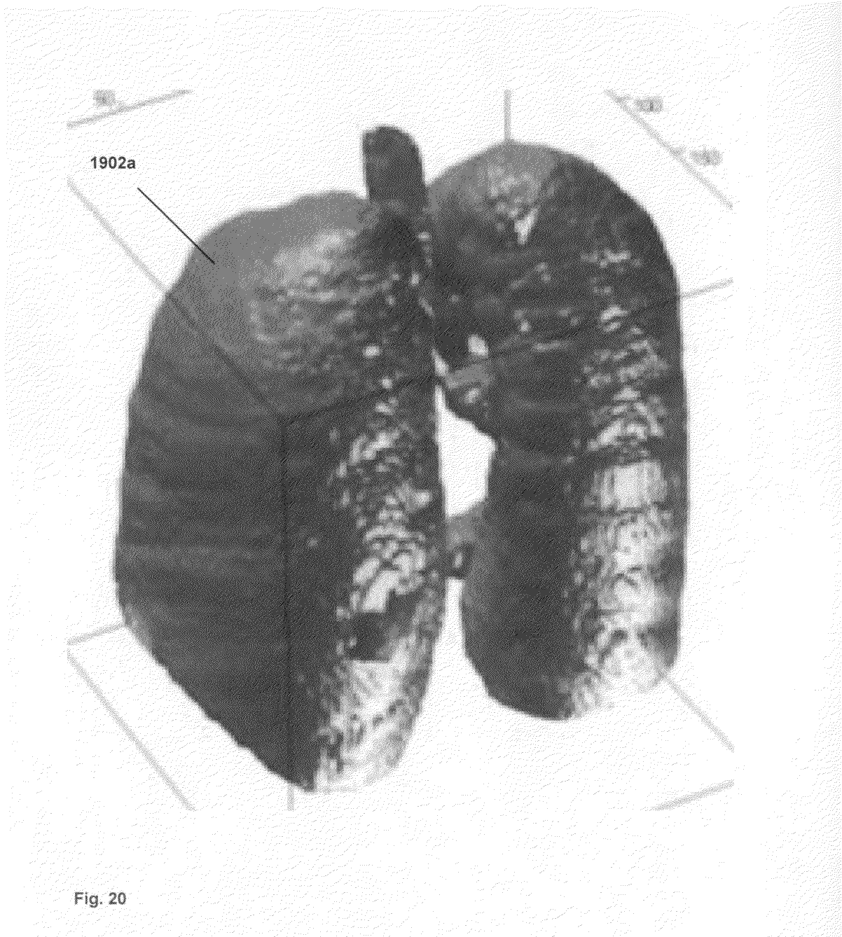

In 1902, lung field segmentation is performed on a series of images. In an exemplary embodiment, lung field segmentation may be performed using a joint segmentation and registration method to thereby extract the lung field by first removing the background and cavity areas and then performing 3-D morphological clean up in the segmented lung field. In an exemplary experimental embodiment, in 1902, as illustrated in FIG. 20, a segmented lung field 1902a was generated.

In 1904, serial image registration of the images is performed.

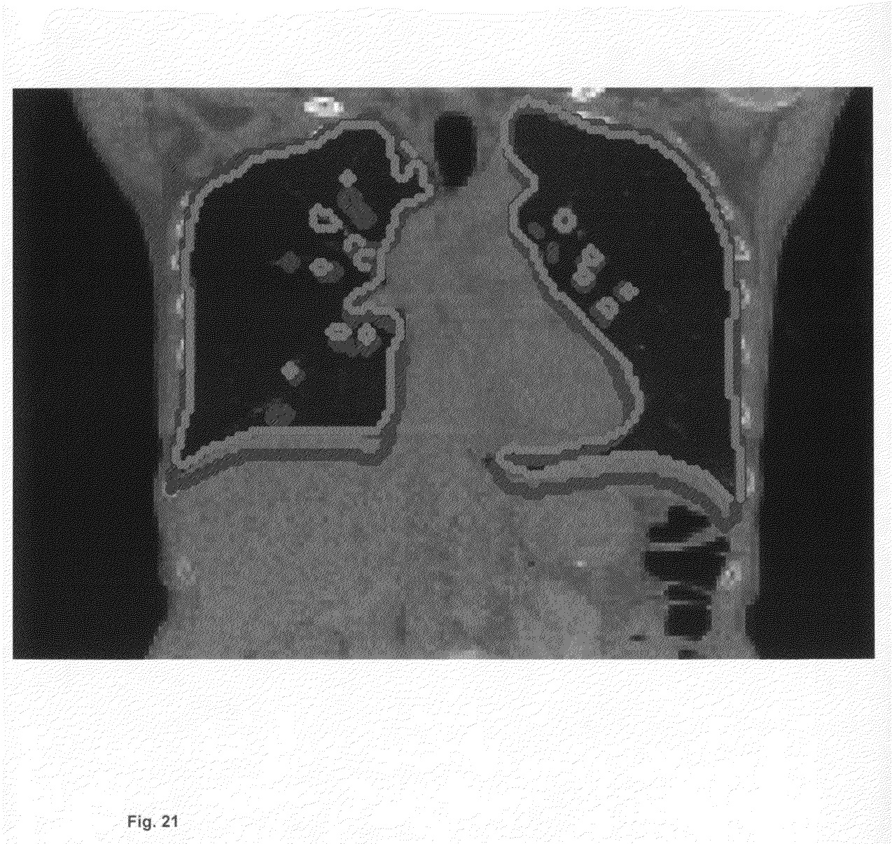

In 1906, registration of the first timepoint image onto a template image is performed. In an exemplary experimental embodiment, in 1906, as illustrated in FIG. 21, surfaces extracted from a 7^{th} image before, in blue, and after, in red, registered to the 1^{st} image, shown as the background. It can be seen that the deformable registration tracked respiratory motion well.

In 1908, the normalized lung field motion vectors and the corresponding fiducial motion vectors are extracted.

In 1910, a lung motion statistical model is constructed by using a kernel principal component analysis (“K-PCA”) on the surface motion vectors.

In an exemplary embodiment, K-PCA is a nonlinear statistical modeling method that can capture the variations of shapes more accurately than PCA. The basic idea is that PCA computed in a high-dimensional implicit mapping function φ(ν), or the feature space, of the surface motion vector ν can be replaced as a PCA of the kernel matrix. Let K denotes the kernel matrix of N sample surface motion vectors, i.e., k_{i,j}=k(νᵢ,νⱼ), K-PCA can be computed in a closed form by finding the first M eigenvalues υᵢ and eigenvectors aᵢ of K, i.e., KA=AV. The corresponding eigenvectors in the feature space can also be computed by multiplying the mapping function values of the samples with A and preserve the variance of data in the feature space. Therefore, given a surface motion vector ν, it can be projected onto the K-PCA space as,

λ=A^{T}(k− k̅), (14)

where k̅ is the mean of kernel vectors, and k is the kernel vector of ν, i.e., kᵢ=k(ν,νᵢ), i=1, . . . N. Because in K-PCA the feature space is induced implicitly, reconstruction of a new vector ν given a feature λ is not trivial, which can be defined in many ways, and different cost functions will lead to different optimization problems.

In 1912, a lung motion estimation model is trained using a least squared support vector machine (“LS-SVM”) to model the relationship between the fiducial signals lung motion feature vectors on the K-PCA space.

In an exemplary embodiment, in 1912, the goal of motion estimating is to establish the relationship between the lung field surface motion ν (represented by a in the K-PCA space) with the fiducials' motion ν^{(d)}. Therefore, given N training sample-pairs {(νᵢ^{(d)},λᵢ)}, i=1, . . . , N, the relationship between fiducial νᵢ^{(d)} and lung field motion feature vector λᵢ needs to be established. In this work, we employ the ridge regression method with the LS-SVM model. Given the time series of the motion vectors λ_{i,t}, i=1, . . . , N; t=1, . . . , T and those of the fiducial motion vectors ν_{i,t}^{(d)}, the goal is to estimate the motion estimation function, λ(t)=θ(ν(t))+e(t), where e is a random process with zero mean and std νₑ². Because the elements of λ(t) are independent each other in the K-PCA space, we can use the least squares support vector machines (“LS-SVM”) model to estimate each element of λ(t). Denoting λ as one element of λ at time t, we can estimate it using:

λ=w^{T}φ(ν^{(d)})+b, (15)

where φ( ) denotes a potential mapping function. w is the weights, and b is the shifting values. The regularized cost function of the LS-SVM is provided in a conventional manner,

$${min}_{w , b , e} ξ ( w , e ) = \frac{1}{2} {w}^{T} w + \frac{γ}{2} {\sum }_{i = 1}^{N} {‖ {e}_{i} ‖}^{2} \\ s . t . {λ}_{i} = {w}^{T} φ ( {v}_{i}^{( d )} ) + b + {e}_{i} , i = 1 , … , N . \qquad ( 16 )$$

γ is referred to as the regularization constant. This optimization actually corresponds to a ridge regression in feature space. The Lagrangian method is utilized to solve the constrained optimization problem, and hence the new cost function becomes:

$$ζ ( w , b , e ; α ) = ξ ( w , e ) - {\sum }_{i = 1}^{N} {α}_{i} ( {w}^{T} φ ( {v}_{i}^{( d )} ) + b + {e}_{i} - {λ}_{i} ) , \qquad ( 17 )$$

with αᵢ as the Lagrange multipliers. The conditions for optimality are equivalent to the following linear equation:

$$[ \begin{matrix}0 & {1}_{N}^{T} \\ {1}_{N} & Ω + {γ}^{- 1} {I}_{N}\end{matrix} ] [ \begin{matrix}b \\ α\end{matrix} ] = [ \begin{matrix}0 \\ λ\end{matrix} ] , \qquad ( 18 )$$

where Λ=λ₁, . . . , λ_{N}]^{T} is the vector formed by the N samples of an element of vector λₜ, 1_{N}=[1, . . . , 1]^{T}εR^{N}, Ω_{i,j}=π(ν_{i,t}^{(d)},ν_{j,t}^{(d)})=φ(ν_{i,t}^{(d)})^{T}φ(ν_{j,t}^{(d)}∀i, j=1, . . . , N with π as the positive definite kernel function. Notice that because of the kernel trick, the feature mapping φ( ) is never defined explicitly, and we only need to define a kernel function π(•,•) of the fiducial vectors. The typical radial basis function (“RBF”) kernel π(ν_{d}ⁱ,ν_{d}^{j})=exp(−∥ν_{d}ⁱ−ν_{d}^{j}∥²/σ²), where σ denotes the bandwidth of the kernel. After solving Eq. (18), we get α and b, and the element of lung motion feature vector λ can be calculated for given fiducial motion vector ν^{(d)}:

$$λ = {\sum }_{t = 1}^{N} {α}_{t} Π ( {v}^{( d )} , {v}_{i}^{( d )} ) + b . \qquad ( 19 )$$

Notice that because different elements of the lung motion feature vector λ are independent, all of the elements of at different timepoints are calculated by this model separately, similar to model the, motion according to different lung capacity.

In 1914, respiratory signals of a patient are transferred onto the template space in order to use the motion estimation model to estimate lung motion feature vectors and reconstruct the lung surface motion vectors of the patient.

In 1916, serial deformations are generated using the surface motion vectors as constraints in a serial deformation simulator.

In 1918, the serial deformation fields are transformed onto the subject space to generate the serial images for visualization during treatment of the patient.

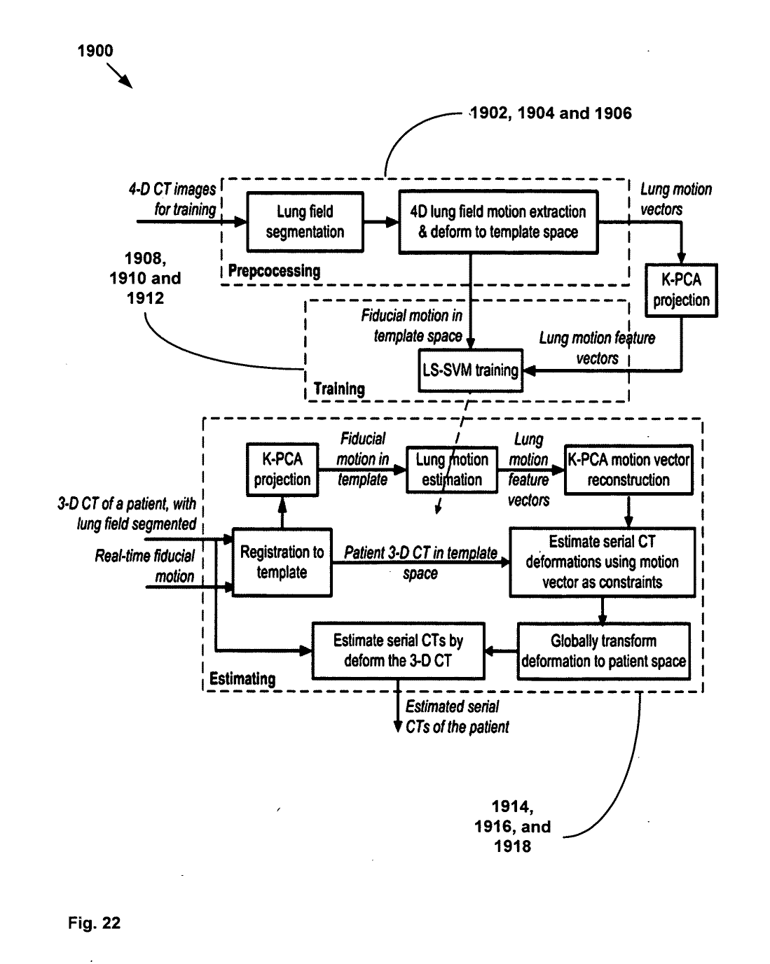

Thus, as illustrated in FIG. 22, the method 1900 provides a motion correction method for use with 4-D CT scans of lung tissue, that includes the estimation of a lung motion model that may then be used to correct for lung motion on a real time basis during treatment of a patient.

In an exemplary embodiment, the method 1900 includes preprocessing, in 1902, 1904 and 1906, including lung field segmentation, serial image registration for lung motion estimation, and registration of the first time-point image of, different subjects onto a template image.

In an exemplary embodiment, the method 1900 includes a training stage, in 1908, 1910, and 1912, in which the normalized lung field surface motion vectors and the corresponding fiducial motion vectors of each subject are extracted, Kernel PCA is performed on the surface motion vectors to construct the lung motion statistic model and reduce the dimensionality for surface points, and a lung motion estimation model is trained using the least squared support vector machine (“LS-SVM”) algorithm to model the relationship between fiducial signals and lung motion feature vectors projected on the K-PCA space.

Finally, in an exemplary embodiment, the method 1900 includes an estimation stage, in 1914, 1916 and 1918, in which an intra-procedural 3-D CT and real-time tracked fiducial signals of a patient are available. The respiratory signals of the patient can be transferred onto the template space in order to use the motion estimation model to estimate the lung motion feature vectors and reconstruct the lung motion vectors, surface motion vectors, of the patient. Serial deformations are generated by using the surface motion vectors as constraints in a serial deformation simulator. The serial deformation fields are finally transformed onto the subject space to generate the serial CT images for online visualization during intervention.

In an exemplary embodiment of the method 1900, if the baseline image of the patient to be tested is Iₜ^{(p)}, we can first register the baseline image onto the template image T using deformable registration Φ_{T-P}:T→I₁^{(p)}, and Φ_{T-P}={G,f} consists of both global G and deformable f components of the registration. The corresponding lung field surface of the patient ν₁^{(p)} can also be aligned onto the template as ν₁. Similarly, the fiducial movement νₜ^{(d,p)} can be aligned onto the template space, denoted as νₜ^{(d)}. Notice that global transformation G needs to be applied to the fiducial motion because we are dealing with different spaces. We can then use Eq. (19) to estimate the serial lung motion feature vectors and reconstruct the lung field motion from K-PCA space to the template image space, denoted as νₜ=2, . . . , T. A conventional lung field motion vector-constrained deformation simulation method is then applied to generate the serial deformation fields. Finally, the deformations are transformed onto the subject space using Φ_{T-P}. The values of these deformation vectors are subject to the global transformation G also.

In several exemplary experimental embodiments of the method 1900, thirty 4-D CT datasets from thirty different patients were processed using the method. In the exemplary experimental embodiments, twelve 3-D images were acquired for each patient, and the images were aligned so that the first and the last images were exhale data and the 7^{th} data was the inhale data. All of the images had an in-plane resolution of 0.98×0.98 mm and a slice thickness of 1.5 mm. To ensure consistent lung field surface representations, one image was randomly selected as the template, and the lung field surface for the selected template image was constructed first. Then, image segmentation and registration was applied to deform the lung field surface of the template image onto all the other images. In this way, we obtained lung field surface correspondences across different subjects and different time-points to ensure that the surfaces had the same trajectory. Using the same strategy, artificial fiducials were automatically put onto the surface of the chest/belly of each CT image. We used a leave-one-out method to evaluate the proposed algorithm. Each time the baseline images from twenty eight subjects were registered onto the template image for training the lung motion estimation model. Then, the baseline image of the left-out subject and the fiducial movement signals of the left-out subject were used to estimate the serial CT images.

In the exemplary experimental embodiments of the method 1900, the errors between the estimated lung field surface and the actual surface at each time point as well as the volumes of the lung fields were used to evaluate the accuracy of the estimation. The procedure was iterated 29 times with one subject left out each time after selecting one image as the template. The following equation was used to calculate the prediction errors for lung field surfaces:

$${Δ}_{i} = \frac{1}{28} {\sum }_{28 tests} dist ( v , \hat{v} ) , subject i is left out . \qquad ( 20 )$$

where the distance between two surfaces is defined as the average of distances from all the points in one surface to the other surface. Another quantitative measure is the volume of the lung. Because the lung fields from both estimated CT and the original CT images were available, we simply calculated the lung volumes and compare whether they are quantitatively close.

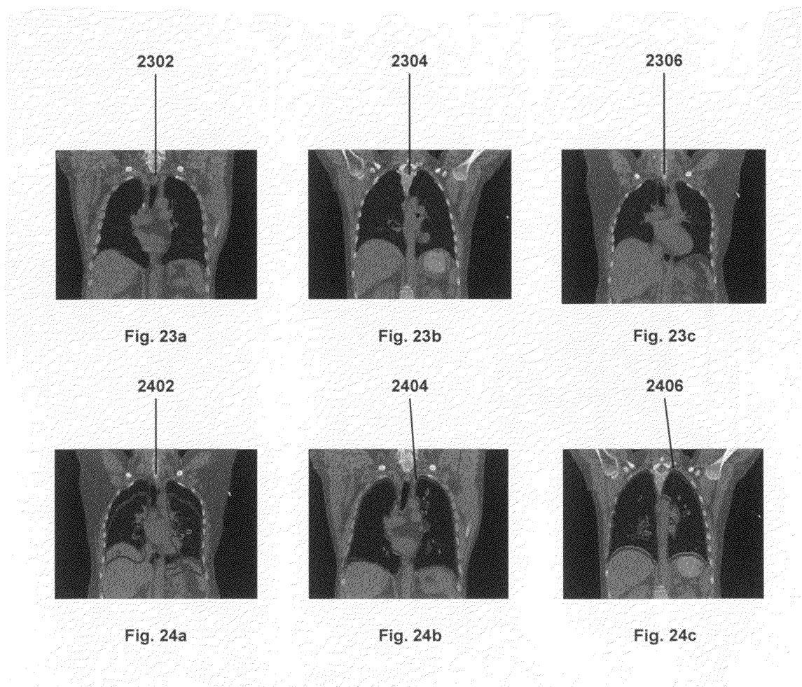

In several exemplary experimental embodiments of the method 1900, as illustrated in FIGS. 23a, 23b, and 23c, exhalation images, 2302, 2304 and 2306, were obtained.

In the several exemplary experimental embodiments of the method 1900, as illustrated in FIGS. 24a, 24b, and 24c, inhalation images, 2402, 2404 and 2406, corresponding to the exhalation images, 23a, 23b, and 23c, respectively, which also illustrated the predicted lung field in blue contours and the actual lung field position was illustrated in red contours.

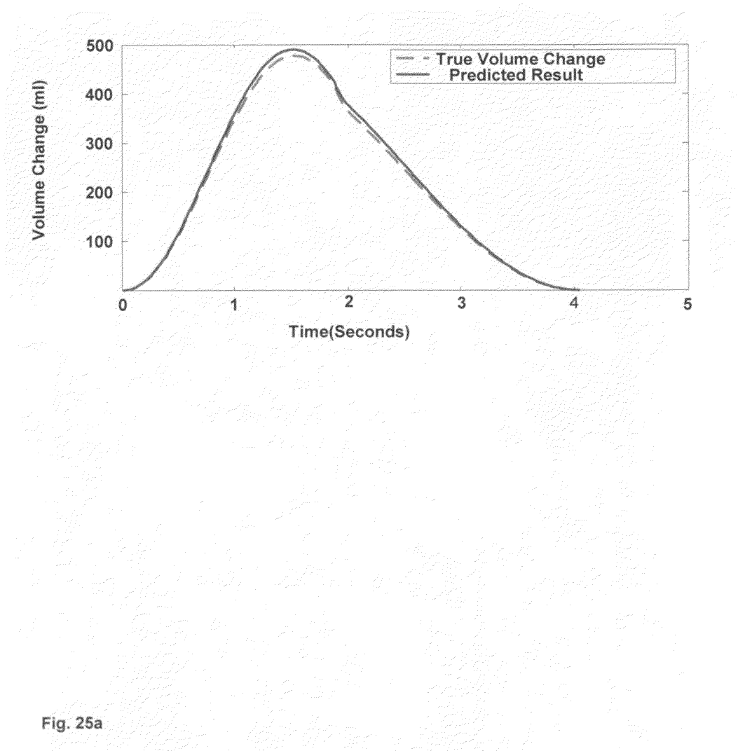

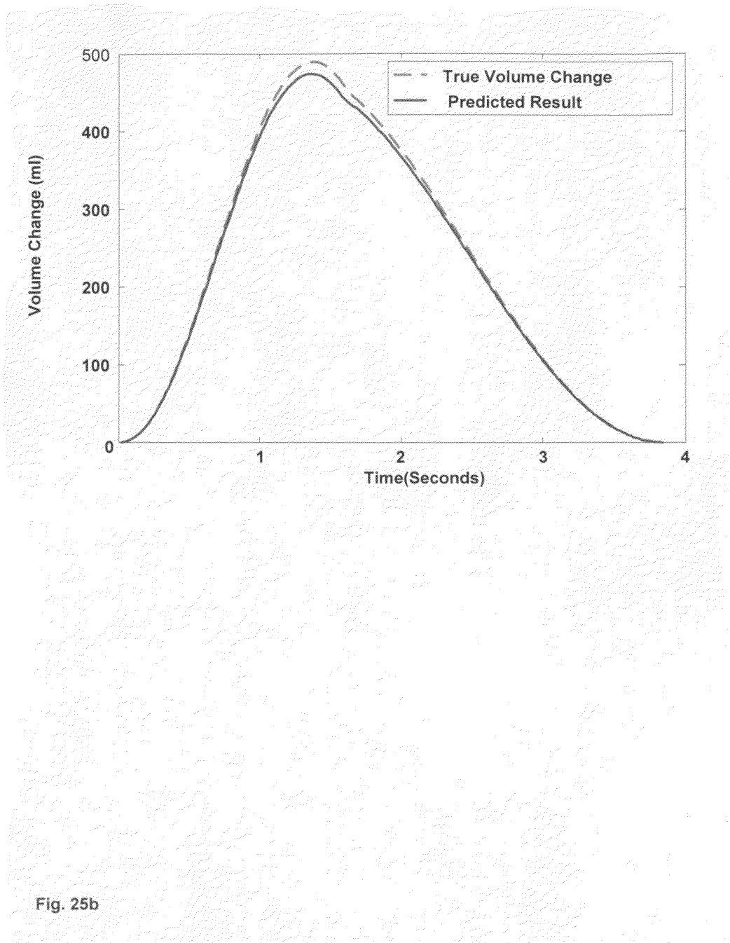

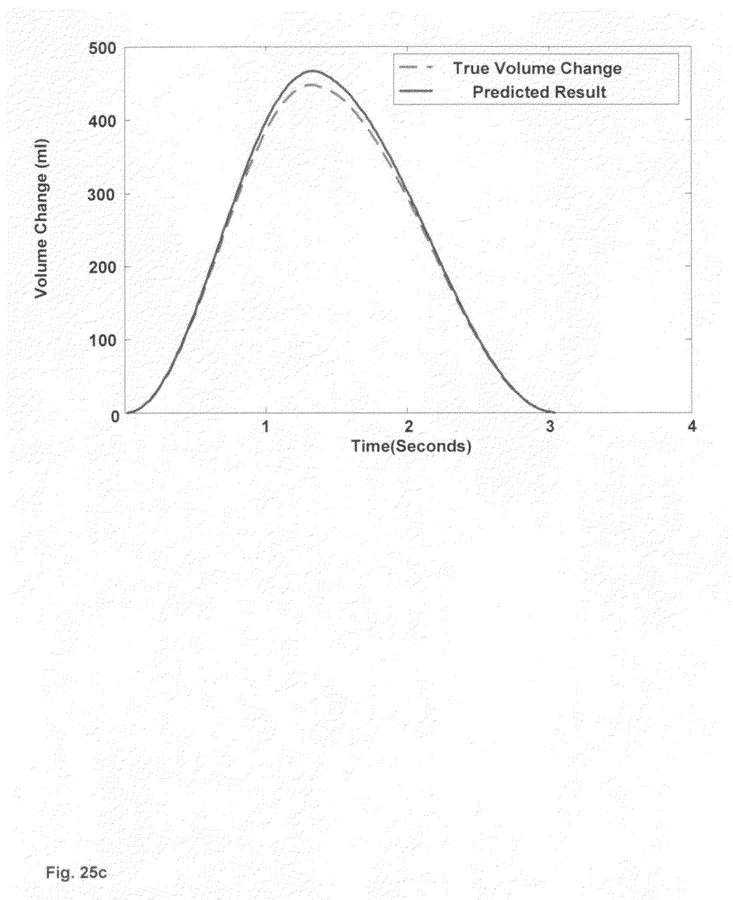

In the several exemplary experimental embodiments of the method 1900, as illustrated in FIGS. 25a, 25b, and 25c, the predicted and actual changes of lung volumes corresponding the corresponding to the exhalation images, 23a, 23b, and 23c, respectively, were generated which indicated high accuracy between the true volume change during breathing and the predicted results. This was an unexpected result.

In the several exemplary experimental embodiments of the method 1900, in order to further validate the experimental results, the average lung field estimation errors for 8 experiments were calculated using Eq. (20), and each result was tested on the left-out subject image as detailed below in Table 4. It can be seen below in Table 4 that the average errors over the serial images are between 1.22 mm and 2.18 mm with an average of 1.63 mm. Overall, an acceptable range of errors were obtained for predicting the lung motion. This was an unexpected result.

TABLE 4
Average errors for lung motion estimation using
leave-one-out validation (units in mm).

| Time | T2 | T3 | T4 | T5 | T6 | T7 | T8 | T9 | T10 | T11 | T12 | Mean |
|---|---|---|---|---|---|---|---|---|---|---|---|---|
| Pat. 1 | 1.20 | 1.39 | 1.61 | 1.64 | 2.69 | 3.49 | 2.75 | 2.75 | 2.88 | 1.94 | 1.63 | 2.18 |
| Pat. 2 | 0.98 | 1.14 | 1.31 | 1.34 | 2.20 | 2.85 | 2.25 | 2.25 | 2.36 | 1.59 | 1.33 | 1.78 |
| Pat. 3 | 1.08 | 1.25 | 1.44 | 1.47 | 2.42 | 3.14 | 2.47 | 2.48 | 2.59 | 1.75 | 1.46 | 1.96 |
| Pat. 4 | 0.83 | 0.96 | 1.11 | 1.13 | 1.86 | 2.42 | 1.90 | 1.91 | 1.99 | 1.34 | 1.13 | 1.51 |
| Pat. 5 | 0.90 | 1.04 | 1.20 | 1.23 | 2.01 | 2.62 | 2.06 | 2.06 | 2.16 | 1.46 | 1.22 | 1.63 |
| Pat. 6 | 0.77 | 0.89 | 1.03 | 1.05 | 1.73 | 2.24 | 1.77 | 1.77 | 1.85 | 1.25 | 1.05 | 1.40 |
| Pat. 7 | 0.67 | 0.78 | 0.90 | 0.92 | 1.51 | 1.96 | 1.54 | 1.55 | 1.62 | 1.09 | 0.92 | 1.22 |
| Pat. 8 | 0.72 | 0.83 | 0.96 | 0.98 | 1.61 | 2.09 | 1.65 | 1.65 | 1.73 | 1.16 | 0.98 | 1.31 |

The present exemplary embodiments of the method 1900 provide an online 4-D CT image estimation approach to patient-specific respiratory motion compensation. The method 1900 provides a motion estimation model that is trained in the template image using a number of 4-D CTs from different subjects. Then, the motion estimation model can be used to simulate serial CTs if a 3-D image and the real-time tracked fiducial signals of a patient are given. Leave-one-out validation results from 30 4-D CT exemplary experimental data showed that an average prediction error of the lung field surface is 1.63 mm. All of the exemplary experimental results of the method 1900 were unexpected results.

In an exemplary embodiment, one or more aspects of the methods 500, 600, 1300, 1400, and 1900 may be used to correction for motion in any sequence of images.

In an exemplary embodiment, one or more aspects of the methods 500, 600, 1300, 1400, and 1900 may be combined, in whole or in part, with one or more other aspects of the methods 500, 600, 1300, 1400, and 1900.

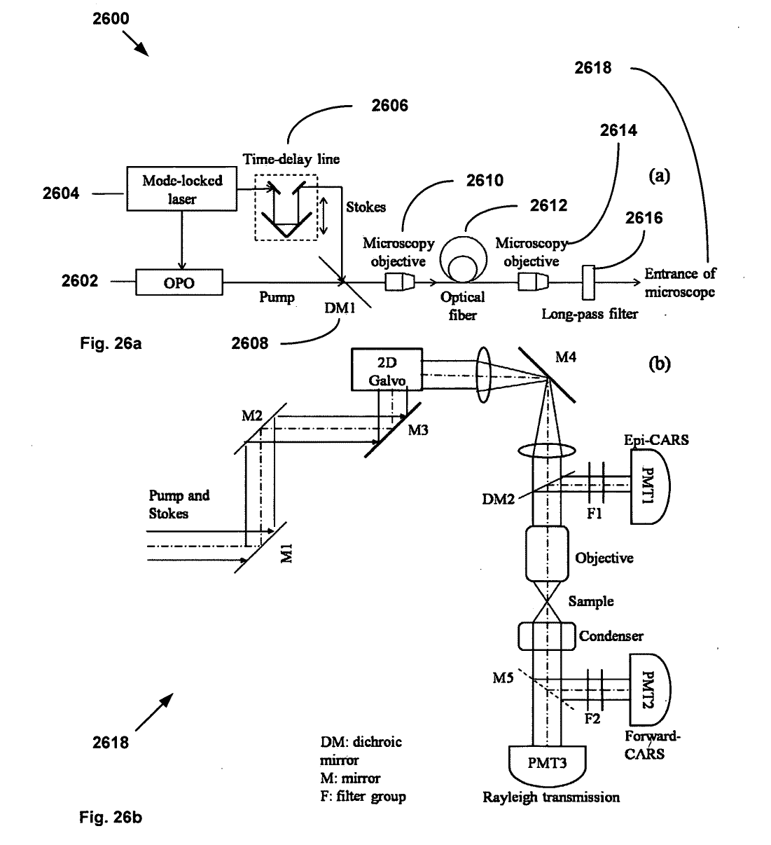

Referring now to FIGS. 26a and 26b, an exemplary embodiment of a CARS microscopy system 2600 includes a light source including a broadly tunable picosecond optical parametric oscillator (“OPO”) 2602 that includes a periodically poled KTiOPO4 crystal, commercially available from Levante, APE, in Berlin, Germany. The OPO 2602 is pumped by the second harmonic, 532 nm, output of a mode-locked Nd:YVO4 laser 2604, commercially available from High-Q Laser, Hohenems, Austria. The laser 2604 delivers a 7-ps, 76-MHz pulse train at both 532 nm and 1064 nm. The 1064 nm pulse train is used as the Stokes wave. The 5-ps OPO signal is used as the pump wave with a tunable wavelength ranging from 670 nm to 980 nm. The beating frequency between the pump and Stokes beams covers the entire chemically important vibrational frequency range of 100-3700 cm⁻¹. The pump and Stokes beams are overlapped by a time-delay line 2606 and dichroic mirror 2608, in both temporal and spatial domains to satisfy the precondition for producing a CARS signal, and then they are conveyed through an objective lens 2610 into and through an optical fiber 2612, another objective lens 2614 and a long-pass filter 2616.

In an exemplary experimental embodiment of the system 2600, the narrow-bandwidth pump and Stokes pulses (˜3.5 cm⁻¹) with durations of 5 ps were able to effectively reduce the non-resonant CARS background, and thus ensured a high signal-to-background ratio as well as a sufficient spectral resolution. This was an unexpected, result. Meanwhile, the light source also provided excellent power stability, allowing high-sensitivity ultrafast imaging.

In the system 2600, the pump and Stokes beams then pass into and through a microscope assembly 2618. In an exemplary embodiment, the microscopy assembly 2618 is modified from a FV300 confocal laser scanning microscope, commercially available from Olympus, in Japan. The modified microscopy subsystem has three PMT detection channels, PMT1, PMT2 and PMT3. The PMT detection channels are able to detect backward (Epi) CARS signals, PMT1, forward CARS signals, PMT2, and Rayleigh scattering transmission signals, PMT3, which was used as a reference.

In an exemplary experimental embodiment of the system 2600, the pump and Stokes beams were coupled into the fiber 2612 using a 10× (NA=0.25, Newport) microscopy objective 2610 and then collimated using another 10× objective 2614. In an exemplary experimental embodiment of the system 2600, a dichroic mirror, DM2, used in the microscope assembly 2618 was 770dcxr, commercially available from Chroma Technology Corp. In an exemplary experimental embodiment of the system 2600, the bandpass filters, F1 and F2, positioned upstream from PMT1 and PMT2, respectively, were hq660/40m-2p optical filters, commercially available from Chroma Technology Corp. In an exemplary experimental embodiment of the system 2600, an objective lens 2618a was used in the microscope assembly that was a 1.2-NA water immersion objective lens (×60, IR UPlanApo, Olympus, Melville, N.J.) was used, yielding a CARS resolution of ˜0.4 μm in the lateral plane and ˜0.9 μm in the axial direction.

In an exemplary experimental embodiment of the system 2600, the optical fiber 2612 was a standard Corning SMF28 communication fiber. The SMF28 communication fiber worked as an MMF below its cutoff wavelength of ˜1260 nm, and covered the CARS operating wavelength range (i.e. from 500 nm to 1100 nm). The SMF28 communication fiber had a core diameter of ˜9.2 μm and a NA of 0.14. Because the V-parameter of the SMF28 communication fiber was ˜4.34 for 817 nm (pump) and ˜3.33 for 1064 nm (Stokes), there were approximately 9 core modes for 817 nm and 6 core modes for 1064 nm based on calculated results from a estimation equation (N≈V²/2). In an exemplary experimental embodiment of the system 2600, the coupling efficiency was about 72% for the pump (817 nm) and 64% for the Stokes (1064 nm), which were coupled into the SMF28 communication fiber using a 10× objective. In an exemplary experimental embodiment of the system 2600, the autocorrelator used to measure auto/cross-correlation function curves was an autocorrelator for APE Levante Emerald OPO (High-Q Laser, Hohenems, Austria). In an exemplary experimental embodiment of the system 2600, the optical spectrometer used to measure the optical spectra was 86142B optical spectrum analyzer (Agilent Technologies Corp., USA) and HR4000 (Oceanoptics Inc., USA).

In an exemplary experimental embodiment of the system 2600, we analyzed the fiber design using several parameters: dispersion length L_{D} (length over which the duration of a pulse width is broaden by √2), walk-off length L_{w} (length over which two pulses at two different wavelengths are separated in time by one pulse duration), nonlinear length L_{NL} (length over which the SPM-induced phase shift of a pulse equals 2π), and average threshold power, P_{2π}, for SPM (power at which the SPM-induced phase shift of a pulse equals 2π, and it can be derived from L_{NL}). In an exemplary experimental embodiment of the system 2600, we considered only step-index fibers for simplicity. These parameters were estimated using equation (1a) to (1d) as follows:

$$\begin{matrix}{L}_{D} = ( - \frac{2 π c}{{λ}^{2}} ) ( \frac{{t}_{P}^{2}}{D} ) , & ( 21 a ) \\ {L}_{W} = \frac{{t}_{P}}{{v}_{g}^{- 1} ( {λ}_{1} ) - {v}_{g}^{- 1} ( {λ}_{2} )} , & ( 21 b ) \\ {L}_{NL} = \frac{π {D}_{eff}^{2} λ {f}_{P} {t}_{P}}{4 {n}_{2} {P}_{ave}} , & ( 21 c ) \\ {P}_{2 π} = \frac{πλ {f}_{P} {t}_{P} {D}_{eff}^{2}}{4 {n}_{2} L} , & ( 21 d )\end{matrix}$$

where tₚ was the pulse width, D was the dispersion of the fiber waveguide, c was the speed of light, λ was the central wavelength of the pulse, v_{g} was the group velocity of the fiber mode [v_{g}=c/(n−λ·dn/dλ), n is effective index of fiber modes, n₂ was the nonlinear refractive index of the fiber material [n₂=2.6×10⁻¹⁶ cm²/W for silica, Pₐᵥₑ was the average power of the pulse in the fiber, D_{eff} was the effective mode diameter, and fₚ was the repetition rate of the laser (76 MHz in our study).

In an exemplary experimental embodiment of the system 2600, the pump and Stokes wavelengths were tuned to 817 nm and 1064 nm, respectively, resulting in a 2845 cm⁻¹ Stokes shift, which matched with the Stokes shift of the CH₂ stretch vibration in lipids.

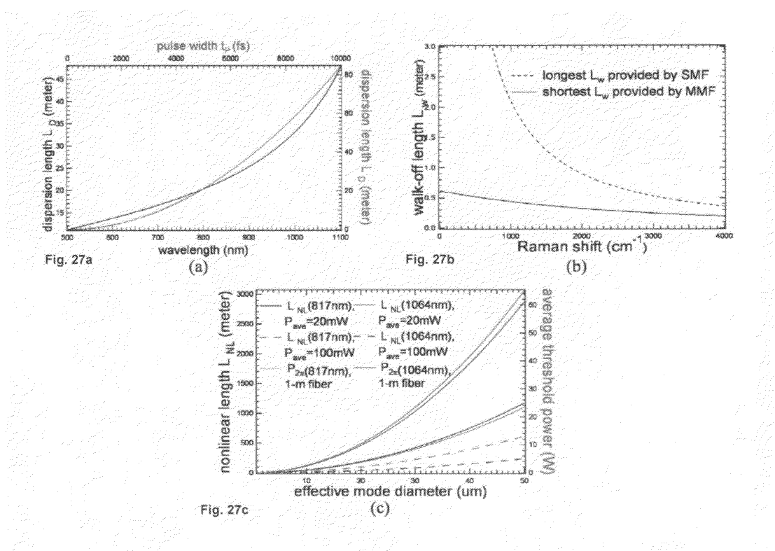

In an exemplary experimental embodiment of the system 2600, one concern in the design of a fiber delivery system was the broadening of pulse width due to dispersion from the fiber. Pulse broadening leads to a lower effective peak power and would decrease the excitation efficiency of CARS. Although, a pre-chirp unit or a piece of dispersion compensation fiber can be used to compensate for dispersion, the wavelength-dependence of dispersion still makes it complicated to achieve the optimal design of a compensation unit. In CARS imaging systems, the typical wavelength of interest ranges from 500 nm to 1100 nm, covering wavelengths from the anti-Stokes wave to the Stokes wave. The typical pulse width of interest ranges from 10 fs to 10 ps. FIG. 27a shows calculated L_{D} as a function of wavelength (black curve, tₚ=5 ps) and pulse width (red curve, λ=817 nm) in the typical ranges using equation (21a), respectively. D was calculated using an equation in the datasheet of the Corning SMF28 Optical Fibers [D=S₀/4(λ−λ₀⁴/λ³), zero dispersion slope S₀=0.086 ps/(nm²·km), zero dispersion wavelength λ₀=1310 nm]. Since the equation in the datasheet of the Corning SMF28 Optical Fibers only covered the intramodal chromatic dispersion (material dispersion and waveguide dispersion) of the fiber but not intermodal dispersion, the intermodal dispersion was simulated using a conventional form of Sellmeier's equation and the step-index fiber model. It was noticed that L_{D} increased nonlinearly with wavelength or pulse width. At tₚ=5 ps, the shortest L_{D} was about 11.2 meters at 500 nm, which was much longer than the physical fiber length needed for a fiber delivery system. Hence, the dispersion issue was negligible in the exemplary experimental embodiment of the system 2600. Meanwhile, L_{D} was about 1 meter for tₚ=1.08 ps at λ=817 nm. This result suggested that the dispersion was not an issue when the pulse width was longer than one picosecond for the SMF28 communication fiber.

In an exemplary experimental embodiment of the system 2600, to calculate the walk-off length L_{w}, a conventional form of Sellmeier's equation was employed to simulate the refractive index of the fiber core and the fiber cladding with 1% index difference. L_{w} defined the extra time delay needed to add to the pump or the Stokes waves to keep their temporal overlapping and to generate CARS signals. Possible shortest and longest L_{w} induced by the group velocity difference between the pump and the Stokes waves were estimated as well. Because the effective indices of fiber modes is lower than the core index and higher than the cladding index regardless of SMFs or MMFs, the core and cladding index could be used to estimate the largest and smallest effective index of the fiber modes. In addition, because the wavelengths of interest were in the normal dispersion region (group index decreases as wavelength increases), the group velocity v_{g} of the fiber mode increased with wavelength. Since the Stokes wavelength is longer than the pump wavelength, v_{g} of the Stokes wave was greater than that of the pump wave. Therefore, when v_{g} of the Stokes and pump waves reached their own maximum and minimum respectively, the largest group velocity difference would be obtained and would result in the shortest L_{w}. Based on our experimental simulations of the system 2600, when the core and cladding indices were used as the effective indices of the pump wave and Stokes wave separately, we could reach the largest group velocity difference between the pump and Stokes waves for MMFs and thus the shortest L_{w}. On the other hand, SMFs possessed the longest L_{w} because there was only a fundamental mode in the fiber and no intermodal dispersions. Based on our simulations of the system 2600, the cladding index was used as the effective index of the fundamental mode to reach the longest L_{w}. The shortest and longest L_{w} were calculated as a function of Raman shift with respective to the Stokes wave, which was assumed to be 1064 nm with tₚ=8 ps (for comparison to experimental results). FIG. 27b shows exemplary simulated results of the system 2600 using equation (21b), where the x-axis Raman shift wave number was limited to 4000 cm⁻¹. In addition, L_{w} was only plotted up to 3 meter to cover practical fiber delivery interest. It was noted that L_{w} decreased as Raman shift increased, and SMFs provided a much larger L_{w} than MMFs, especially for Raman shifts within 1200 cm⁻¹. For practical SMFs and MMFs, L_{w} fell in the region between the shortest L_{w} curve and the longest L_{w} curve. Compared to the single L_{w} curve of SMFs, L_{w} of MMFs existed as an area between the two curves due to cross-calculations of L_{w} between different modes. Hence, MMFs had a larger delay adjustment range than SMFs to obtain the optimal temporal overlapping of pump and Stokes waves (i.e. SMFs had an optimal delay adjustment point, while MMF may have an optimal delay adjustment range instead). This effect was confirmed in our experiments with the system 2600 using SMF and MMF. To increase L_{w}, laser pulses with larger tₚ can be used since L_{w} is linearly proportional to tₚ. Conversely, L_{w} will be much shorter when fs laser pulses are used for CARS imaging. Additionally, because the core index (i.e. upper limit of effective index) and the cladding index (i.e. lower limit of effective index) were used as the largest and smallest effective index of fiber modes in our simulations, the shortest L_{w} presented in FIG. 27b reached the limiting point for MMF and thus was independent of the number of modes propagating in MMF.

In an exemplary experimental embodiment of the system 2600, for fiber delivery of ultrafast laser pulses, nonlinear effects (e.g. SPM and FWM) are critical issues, which could either reshape the spectra of laser pulses or generate new laser frequencies. To address these concerns, we estimated SPM induced nonlinear length L_{NL} and average threshold power P_{2π} which were induced by SPM in this section. The FWM effect was also investigated in our experiments due to the complicity and significance to estimate FWM originated from interactions between different core modes in MMF. In our calculations, we estimated L_{NL} as a function of the mode diameter using equation (21c), assuming fₚ was 76 MHz, tₚ was 5 ps when λ was 817 nm and tₚ was 10 ps when λ was 1064 nm. The solid and the dashed curves in FIG. 27c represent different L_{NL} when Pₐᵥₑ=20 mW and Pₐᵥₑ=100 mW, respectively. We noted that L_{NL} increased with the mode diameter quadratically, indicating that the SPM effect would be greatly reduced with the increase of the mode diameter. L_{NL} for Pₐᵥₑ=20 mW was five times larger than that for Pₐᵥₑ=100 mW. In our exemplary experimental embodiments of the system 2600, when the mode diameter was ˜9 μm, L_{NL} for 817 nm at Pₐᵥₑ=20 mW and Pₐᵥₑ=100 mW equaled about 45 meters and 9 meters, L_{NL} for 1064 nm at Pₐᵥₑ=20 mW and Pₐᵥₑ=100 mW equaled about 100 meters and 20 meters. Additionally, we estimated the average threshold power P_{2π} as a function of the mode diameter using equation (21d), assuming fiber length equaled to 1 meter, fₚ was 76 MHz, tₚ was 5 ps when λ was 817 nm and tₚ was 10 ps when λ was 1064 nm. The exemplary experimental results for the system 2600 are plotted in FIG. 27c. Similar to L_{NL}, we noted that P_{2π} increased quadratically with the mode diameter. In FIGS. 27a, 27b and 27c, both L_{NL} and P_{2π} for 1064 nm are larger than those for 817 nm, which was predicted by equations (21c) and (21d).

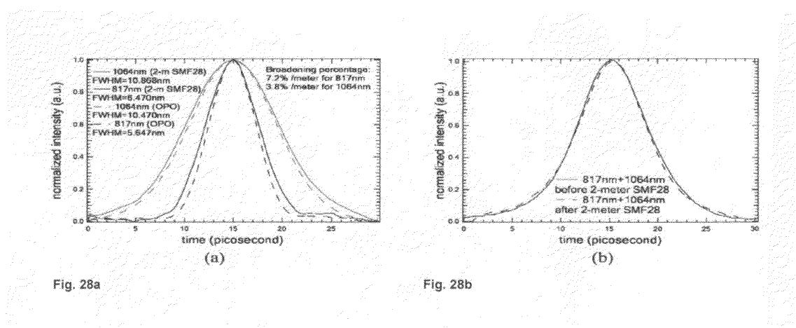

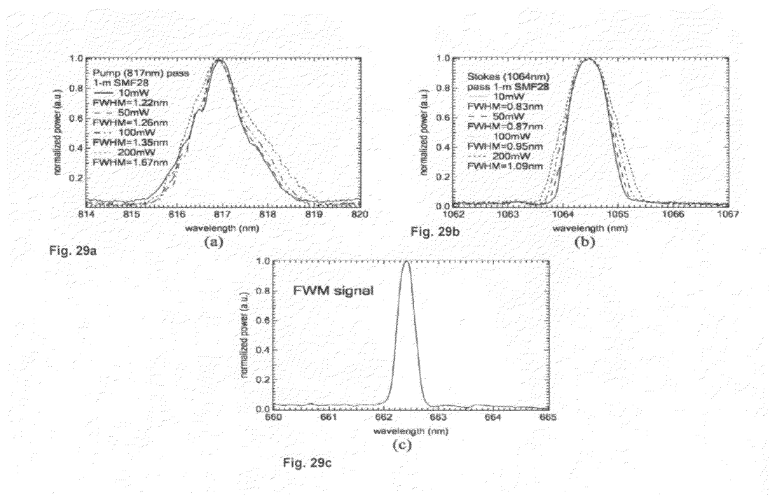

In an exemplary experimental embodiment of the system 2600, we examined the dispersion-induced broadening of the pulse width using SMF28 communication fiber, whose core diameter was 9.2 μm and cladding diameter was 125 μm. We measured the autocorrelation function curves of the pump (817 nm, 50 mW) and the Stokes (1064 nm, 50 mW) waves at two different conditions: (1) direct output from the OPO or laser and (2) after passing through a 2-meter SMF28 communication fiber. The normalized autocorrelation curves were shown in FIG. 28a. By measuring the FWHM bandwidth of the curves, we calculated the percentage of pulse broadening per meter: 7.2% per meter for 817 nm and 3.8% per meter for 1064 nm. Based on the experimental results shown in FIG. 28a (L_{D}=21.07 m for 817 nm, L_{D}=41.24 m for 1064 nm), the calculated percentage of pulse broadening was 6.7% per meter for 817 nm and 3.4% per meter for 1064 nm, which were about 6.9% and 10% smaller than the measured ones. This discrepancy could be caused by the step-index fiber model used in simulations, which utilized weakly-guiding and scalar mode approximations. In addition, the discrepancy could also arise from measurement errors, as well a s possible difference between the real fiber parameters and those used in simulations. In spite of this discrepancy, the broadening effect of the pulses was still negligible by using MMFs for the fiber delivery in CARS imaging.

In an exemplary experimental embodiment of the system 2600, we examined the walk-off length L_{w} by measuring the cross-correlation function curves before and after passing though an 2-meter SMF28 communication fiber. A strong Gaussian-shape single peak of cross-correlation function curve indicated that the pump (817 nm) and the Stokes (1064 nm) waves achieved a good overlapping in time without any walk-off; otherwise there would be small shoulders. After the two waves passed the 2-meter length of the SMF28 communication fiber, we adjusted the translation stage of the delay line to obtain the strongest Gaussian-shape single peak again. Then, we calculated the adjustment amount of the translation stage of the delay line. This amount value corresponded to the walk-off induced delay in distance by the SMF28 communication fiber. The normalized cross-correlation intensity curves, before and after passing though a 2-meter SMF28, are shown in FIG. 28b. Adjustment for the 2-meter length of the SMF28 communication fiber was 1.607 mm or 53.56 ps (26.78 ps/meter). Because the measured FWHM pulse width of 817 nm and 1064 nm pulses were 5.647 ps and 10.47 ps, we took the average (˜8 ps) of the pulse width to calculate the measured L. Then, the measured L_{w} was 0.2987 meters for 8-ps pulse and 2-meter SMF28. Based on our simulated L_{w} in FIG. 28b, the simulated shortest L_{w} for MMF, which was 0.2578 meters for 2845 cm⁻¹ Raman shift, corresponding to the pump (817 nm) and Stokes (1064 nm) waves. Therefore, our measured L_{w} result was longer than the simulated shortest L_{w}, which fell in the region between the simulated longest L_{w} and the simulated shortest L_{w} shown in FIG. 28b.

In an exemplary experimental embodiment of the system 2600, we examined the SPM effect induced by the pump (817 nm) and Stokes (1064 nm) waves propagating in a 1-meter length of the SMF28 communication fiber. Within the power range from 0 to 200 mW used in our experiments, we did not observe the cross-phase modulation (XPM) effect (i.e. no spectral change) when both the pump and Stokes waves propagated in the fiber simultaneously. Hence, we measured the spectrum with either the pump or Stokes wave propagating in the fiber separately. FIGS. 29a, 28b and 29c illustrate normalized measured pump (817 nm) and Stokes (1064 nm) wave spectra as a function of propagating power in SMF28 communication fiber. We noted that there were some ripples in spectra of pump (817 nm) waves. The ripples in spectra of pump waves originated from the artifact effect of the 2^{nd} order grating inside the grating-based Agilent OSA, which was confirmed by changing the grating option in the OSA configuration and the technical supporting staff of the Agilent Corp. Another artifact effect of the grating-based Agilent OSA was that when the input λ<850 nm, the OSA display showed both λ and 2λ, which was described in Agilent OSA application notes and it is important for FWM measurements. In FIG. 29a, we noted that FWHM bandwidth of pump waves increased with power. At 200 mW, the FWHM bandwidth broadened by ˜36.8%, but it was still far from 2π phase shifts (peak splitting in central wavelength). The power of 200 mW exceeded the average power, i.e. less than tens of milliwatts, usually applied in CARS microscopy. In FIG. 29b, we noted that FWHM bandwidth of Stokes waves also increased with power. At 200 mW, FWHM bandwidth broadened by ˜31.3%, which was far from 2π phase shifts as well. The exemplary experimental results illustrated in FIGS. 29a, 29b and 29c indicated that the 1-meter SMF28 communication fiber can be used to deliver individual pump or Stokes waves without generating serious SPM-induced phase shifts. This was an unexpected result.

In an exemplary experimental embodiment of the system 2600, we examined the FWM effect induced by the pump (817 nm) and Stokes (1064 nm) waves simultaneously propagating in a 1-meter long SMF28 communication fiber. We found weak anti-Stokes generations at 663 nm, which matched the 2845 cm⁻¹ anti-Stokes shift of the CH₂ stretch vibration. Therefore, it would result in spurious CARS signals and background noise in the imaging system. The zero-dispersion wavelength of fibers plays an important role in the FWM behavior. For SMF, the FWM phase-matching condition is difficult to be met for the frequency components shifted by more than 3000 cm⁻¹ from zero dispersion wavelength of the fiber. However for MMF, the FWM phase-matching condition is relaxed by the easiness of satisfying the phase-matching condition with pump, Stokes and anti-Stokes waves which propagate in different modes. For instance, the anti-Stokes wave at the fiber mode LP₂₁ can be generated by the combination of the pump (LP₀₁), the pump (LP₀₂) and the Stokes (LP₁₁). As a result, more fiber modes exist in the fiber, more diverse mode combinations will exist to satisfy the FWM phase-matching condition, generating stronger FWM signals. Thus, MMF is more likely to satisfy the phase-matching condition to generate the FWM signals than SMF. In our exemplary experimental embodiments of the system 2600, although the anti-Stokes (663 nm, ˜7450 cm⁻¹) wavelength was far away from the zero dispersion wavelength of the SMF28 (i.e. 1310 nm), SMF28 communication fiber can still easily satisfy the FWM phase-matching condition because there were approximately 9 fiber modes for 817 nm and 6 fiber modes for 1064 nm existing in SMF28. FIG. 29c shows a typical normalized measured FWM (663 nm) wave spectrum output from a 1-meter long SMF28. In our exemplary experimental embodiments of the system 2600, a clear peak was observed at 663 nm while no other new peaks occurred when the pump (817 nm) and Stokes (1064 nm) waves propagated in SMF28 simultaneously. Also, we noted that anti-Stokes signals (663 nm) was quadratically proportional to the input power of the pump (817 nm) and linearly proportional to the power of the Stokes (1064 nm). In an exemplary experimental embodiment of the system 2600, the FWM signal was measured using an optical spectrometer HR4000 (Oceanoptics, Inc), which provided better sensitivity than Agilent OSA in the range of visible wavelengths.

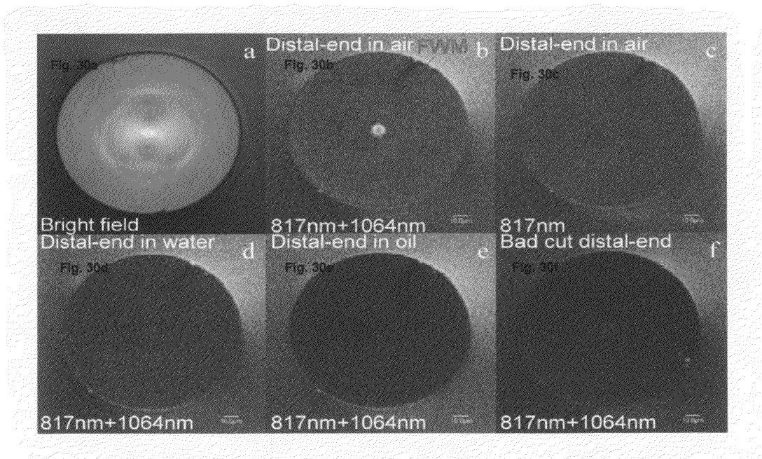

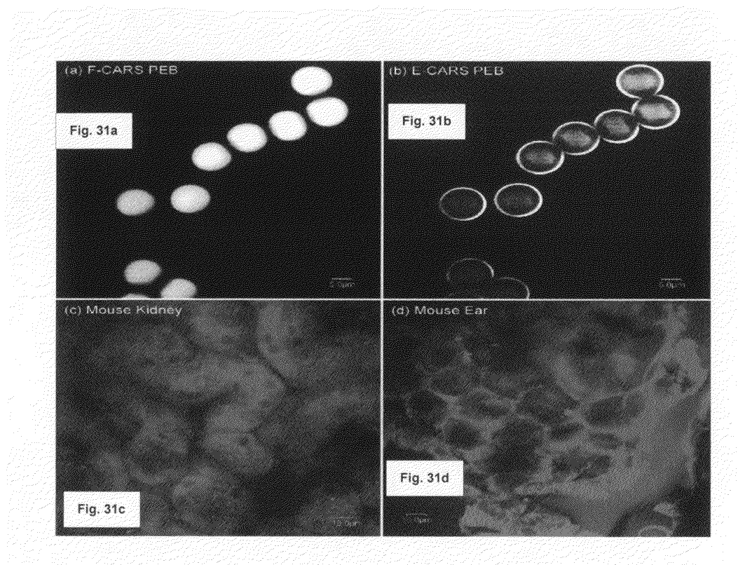

In an exemplary experimental embodiment of the system 2600, we verified the FWM effect by, instead of inserting the 1-meter SMF28 communication fiber 2612 before the entrance of the microscopy assembly 2618, we mounted it directly under the 10× (NA=0.25, Newport) objective and captured the backward FWM (or nonresonant CARS) images from the proximal end of the fiber 2612. The Epi-CARS channel was used with a bandpass filter (hq660/40m-2p, Chroma Technology Corp). Powers of the pump and the Stokes at the proximal end of MMF were 200 mW and 100 mW, respectively. FIGS. 30a, 30b, 30c, 30d, 30e and 30f show a brightheld image of the well-cleaved proximal end of a 1-meter SMF28 and five CARS images from the proximal end of the 1-meter SMF28 communication fiber at various conditions, such as pump plus Stokes, pump only, distal end suspending in air or immersing in water/oil, and well-cleaved or bad-cleaved distal end. We observed strong FWM signals emerging from the fiber core when the pump (817 nm) and Stokes (1064 nm) waves were coupled simultaneously into the SMF28 as shown in FIG. 30b. The circular pattern of the FWM signals indicated the mode distribution in the core of the SMF28 communication fiber was not uniform. No FWM signal was detected when only the pump wave (817 nm) was coupled into the SMF28 as shown in FIG. 30c. There were weak FWM signals when the well-cleaved distal end of the SMF28 was immersed in water, as illustrated in FIG. 30d, or oil, as illustrated in FIG. 30e, or when the distal end was bad-cleaved/cut, as illustrated in FIG. 30f. Collectively, these findings suggested that the FWM signals were mainly generated in the forward direction.

In an exemplary experimental embodiment of the system 2600, we tested the SMF28 communication fiber to deliver ps lasers for CARS imaging. Emerging from the dichroic mirror (DM1), the pump (817 nm) and Stokes (1064 nm) waves were coupled into a 1-meter long SMF28 communication fiber by a 10× Newport objective. After passing the fiber, the two waves were then collimated by another 20× Newport objective. A 750 nm long-pass filter (FEL0750, Thorlabs Inc.) was used to eliminate the FWM (663 nm) signals generated in the SMF28 before the microscopy assembly 2618. The pump and the Stokes waves were tuned to 40 mW and 20 mW for CARS imaging. We characterized the performance of the setup by imaging calibrated 10 μm polystyrene beads (PEB), which generated strong resonant CARS signals at the aliphatic symmetric CH₂ stretch (Δω=2845 cm⁻¹). FIGS. 31a and 31b show the forward and Epi CARS images of 10 μm polystyrene beads spin-coated on a glass slide by delivering ps lasers through a 1-meter SMF28 communication fiber. The CARS images were verified by the results of single pump wave taken at 2845 cm⁻¹. In FIG. 31b, the Epi-CARS image clearly showed the characteristic ring structure of PEBs due to the relative large size of PEBs compared to the small coherence length of Epi CARS signals. In terms of the CARS image quality, such as contrast and resolution, no clear difference was noticed between using free-space and using a 1-meter SMF28 communication fiber for delivery of the laser. To further assess the performance of delivering ps lasers through the SMF28 communication fiber, we imaged two types of mouse tissues ex vivo. FIGS. 31c and 31d show the Epi CARS images of the mouse kidney and ear, respectively. There were no discernable degradations with regard to the image quality obtained through fiber delivery compared to free-space delivery. In addition, we tested the stability of this SMF28 fiber-delivered CARS system by characterizing fluctuations of the CARS signal while imaging PEBs. The fluctuations of the CARS signal were 1.5% and 4.6% for the short-term (i.e. one-hour) and the long-term (i.e. two-day), respectively. These results demonstrated that the SMF28 communication fiber can be used to deliver ps lasers for CARS imaging, and a filter can be used to block the FWM signals generated in the fiber. This was an unexpected result.

In an exemplary experimental embodiment of the system 2600, we found that the dispersion of fibers is not an important issue for the design of a fiber delivery system. The reason is that the dispersion length of the fibers is much longer than the physical length of the fibers used for laser delivery in a CARS imaging system. Because of the group velocity difference between the pump and Stokes waves traveling in the fibers, the delay line needs a certain amount of adjustment to compensate for the walk-off length between the two beams. Based on our analyses on the nonlinear length and the average threshold power for SPM, it suggests that fibers with larger effective mode diameters can be used to decrease SPM-induced phase shifts of the laser pulses. There are FWM signals at the anti-Stokes frequency generated in the SMF28 communication fiber. These signals mainly propagate in the forward direction. A long-pass filter can be used to filter out the FWM signals to eliminate spurious CARS signals and background noise in the system. Thus, according to our exemplary experimental embodiment of the system 2600, multimode SMF28 communication fibers can be used for delivery of picosecond excitation lasers in a CARS imaging system without any degradation of the image quality. This was an unexpected result.

In an exemplary embodiment, one or more aspects of the systems 100, 200, 300, and 400 and one or more aspects of the methods 500, 600, 1300, 1400, and 1900 may be combined, in whole or in part, with one or more other aspects of the system 2600.

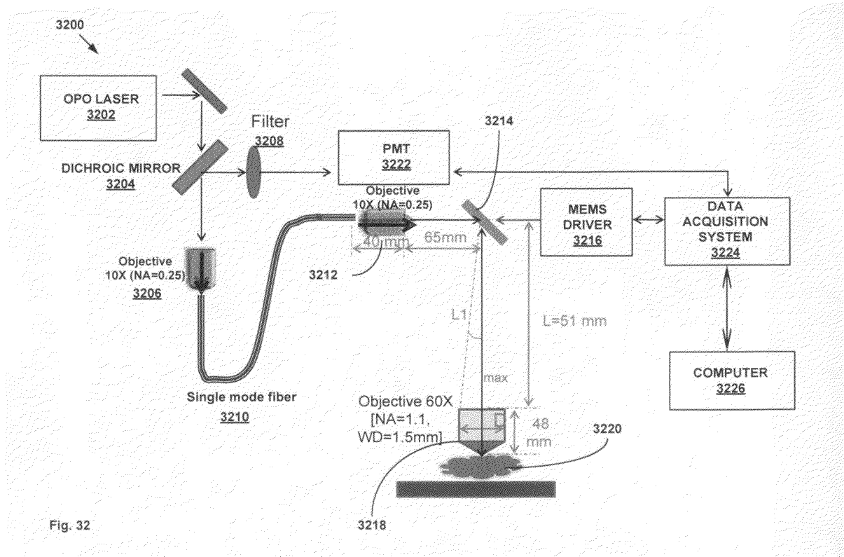

Referring now to FIG. 32, an exemplary embodiment of a CARS microscopy system 3200 includes an OPO laser 3202, for providing a pump and stokes beams, that is operably coupled to a dichroic mirror 3204. The mirror 3204 is also operably coupled to an objective lens 3206 and an optical filter 3208. In an exemplary embodiment, the optical filter 3208 is a long pass filter.

The objective lens 3206 is also operably coupled to an end of a single mode optical fiber 3210. The other end of the fiber 3210 is operably coupled to an objective lens 3212. The object lens 3212 is also operably coupled to a reflective surface of a MEMs mirror 3214 that may be actuated by a MEMs driver 3216. The reflective surface of the MEMs mirror 3214 is also operably coupled to an objective lens 3218 that may be positioned proximate a tissue sample 3220.

The optical filter 3208 is also operably coupled to a PMT 3222 that, in turn, is also operably coupled to a data acquisition system 3224. The data acquisition system 3224 is also operably coupled to a computer 3226.

In an exemplary embodiment, during operation of the system 3200, the system operates substantially the same as one or more of the systems 100, 200, 300, 400 and 2600 as described herein.

In an exemplary experimental embodiment of the system 3200, the length of the lens 3212 was 40 mm, the numerical aperture (“NA”) of the lens 3212 was 0.25, the magnification of the lens 3212 was 10×, the distance between the end of the lens 3212 and the reflective surface of the MEMs mirror 3214 was 65 mm, the distance between the reflective surface of the MEMs mirror 3214 and an end of the lens 3218 was 51 mm, the length of the lens 3218 was 48 mm, the NA of the lens 3218 was 1.1, the magnification of the lens 3218 was 60×, the back aperture of the lens 3218 was 4.96 mm, the distance L1 was 51.24 mm, the laser radius before entering the lens 3218 was 2 mm, and the maximum scanning angle θₘₐₓ of the MEMs mirror 3214 during operation was 2.784 degrees.

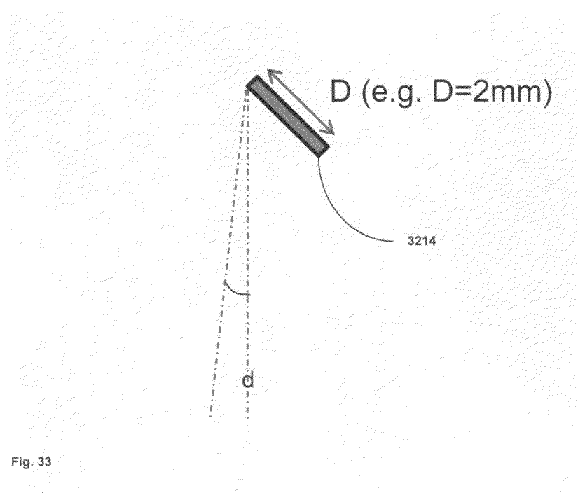

In an exemplary experimental embodiment of the system 3200, as illustrated in FIG. 33, the diffraction angle θ_{d} of the MEMs mirror 3214 during operation was determined to be 0.0117 degrees. As a result, the number of resolvable scanning angles was calculated as 2θₘₐₓ/2θ_{d}=5.569 degrees/0.0234 degrees=237. Thus, the maximum number of scanning steps in 2D by the MEMs mirror 3214 was determined to be 237, which provided a maximum resolution of 237×237 pixels.

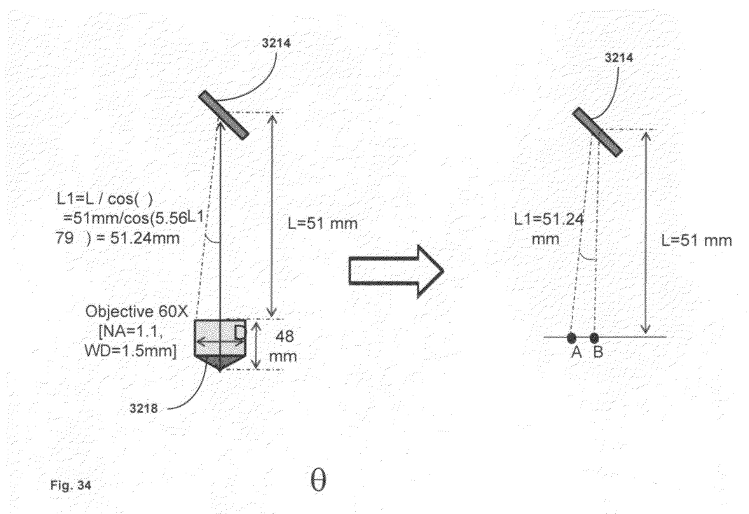

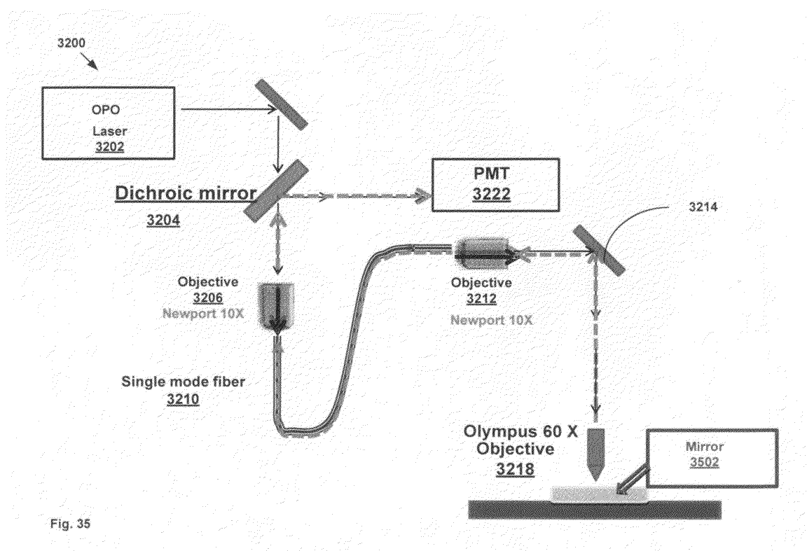

In an exemplary experimental embodiment of the system 3200, as illustrated in FIG. 34, the linear scanning speed v of the MEMs mirror 3214 was calculated as equal to ω×r, where v is the tangential velocity of a point about the axis of rotation of the MEMs mirr, ω is the angular speed of the MEMs mirror, and r is the radius of rotation. As a result of this calculation, it was observed that the operation of the MEMs mirror 3214 may, in an exemplary embodiment, be nonlinear and thus a nonlinear feeback control system may be used to monitor and control the operation of the MEMs mirror. Furthermore, in an exemplary experimental embodiment of the system 3200, the minimum linear scan step of the MEMs mirror 3214 was determined to be 0.021 mm.

In an exemplary experimental embodiment of the system 3200, the effective NA for the lens 3218 was determined to be 0.4433.

In an exemplary experimental embodiment of the system 3200, the resolution of the lens 3218 was determined to be 1124.

In an exemplary experimental embodiment of the system 3200, the laser spot size provided by the lens 3218 was determined to be 2248 nm.

In an exemplary experimental embodiment of the system 3200, as illustrated in FIG. 35, the signal to noise (“S/N”) of the system was determined by positioning a mirror 3502 immediately below the lens 3218. In this manner, the strongest backward signal would be obtained, regardless of CARS or other optical modalities, which would provide the best S/N ratio obtainable by the exemplary experimental embodiment. In the exemplary experimental embodiment of the system 3200, the S/N ratio observed was 3.5.

Thus, in an exemplary embodiment of the system 3200, the resolution of the laser beam output of the lens 3218 was 1124 nm, the laser spot size provided by the lens 3218 was 2248 nm, the maximal scan angle θₘₐₓ of the MEMs mirror 3214 was equal to 2.784 degrees, the number of scanning steps per image was 237, the maximum number of pixels within an image was 237×237, the maximal linear scan speed difference at the back aperture of the lens 3218 was ω×240 μm, and the minimum linear scan step at the back aperture of the lens 3218 was about 21 μm.

Furthermore, in an exemplary embodiment of the system 3200, the maximum field of view provided by the lens 3218 is 233×233 μm²; speed of scanning of 0.496 second/scan line; full scanned image with 237×237 pixels in about 120 second; maximum scan angle θₘₐₓ of 2.784 degrees; the objective lens 3218 is an Olympus LUMFL60XW, near infra-red objective lens, FN of 14, NA of 1.1 and WD of 1.5 mm; and number of pixels per image of 237×237.

In an exemplary embodiment, one or more aspects of the systems 100, 200, 300, 400 and 2600 and one or more aspects of the methods 500, 600, 1300, 1400, and 1900 may be combined, in whole or in part, with one or more other aspects of the system 3200.

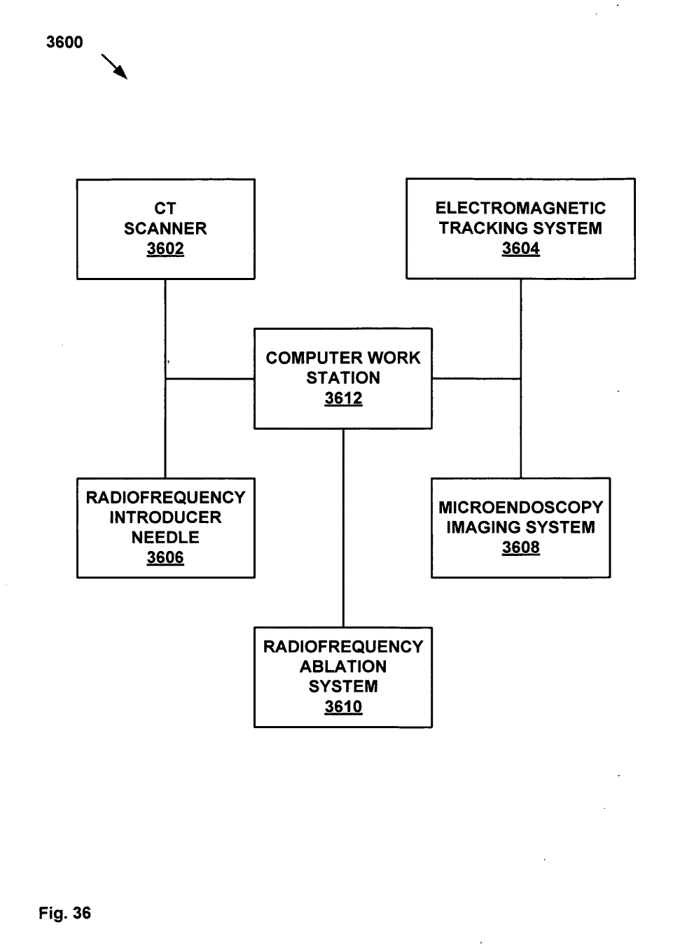

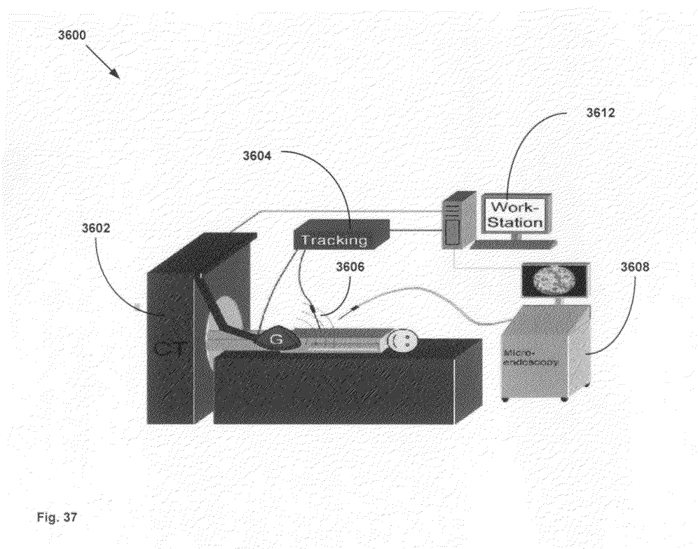

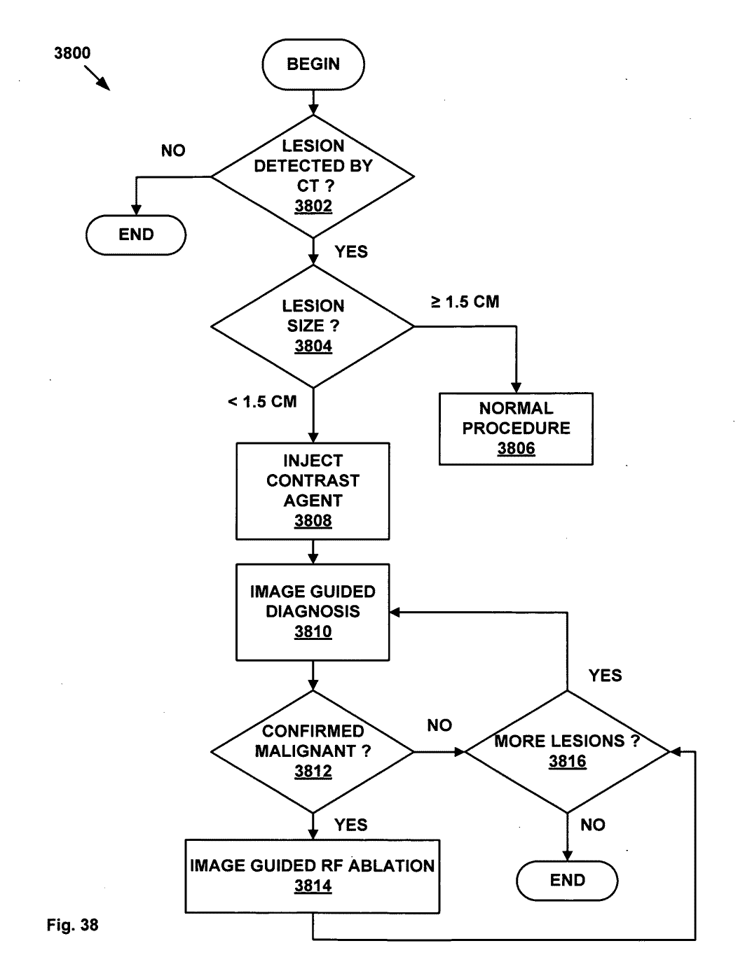

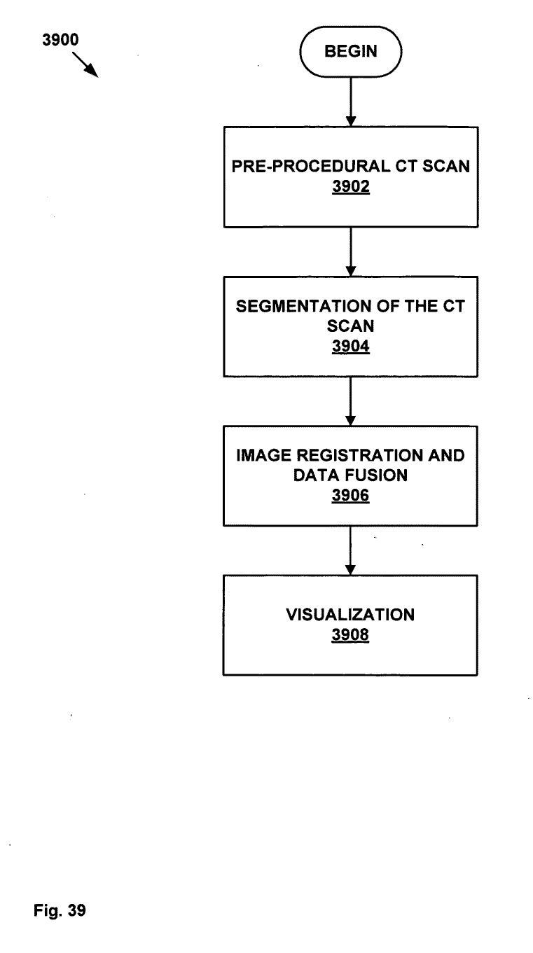

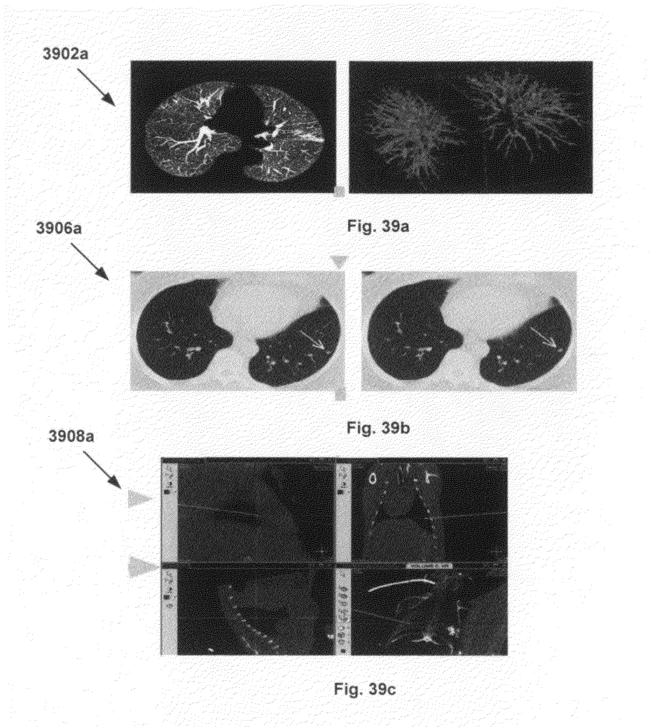

Referring now to FIGS. 36 and 37, an exemplary embodiment of a minimally invasive image-guided (“MIMIC”) molecular imaging system 3600 for patient treatment and diagnosis includes a conventional CT scanner 3602, an electromagnetic (“EM”) tracking system 3604, a radio frequency (“RE”) introducer needle 3606, a microendosopy imaging system 3608, an RF ablation system 3610, and a computer work station 3612 that may be operably coupled to the CT scanner, the EM tracking system, the RF introducer needle, the microendosopy imaging system, and the RF ablation system.

In an exemplary embodiment, the EM tracking system 3604 may be a conventional EM tracking system such as, for example, an NDI Aurora EM tracking system.

In an exemplary embodiment, the RF introducer needle 3606 and the RF ablation system 3610 may be a conventional RF introducer needle and RF ablation system such as, for example, a Valleylab Cool-tip ablation system.

In an exemplary embodiment, the microendosopy imaging system 3608 may be a conventional microendoscopy system, a conventional CARS microendoscopy system, and/or may include one or more aspects of the systems 100, 200, 300, 2600 and 3200 and/or the methods 500, 600, 1300, 1400, and 1900.

In an exemplary embodiment, the computer work station 3612 is operably coupled to the CT scanner 3602, the EM tracking system 3604, the radio frequency introducer needle 3606, the microendosopy imaging system 2608, and the RF ablation system 3610 for monitoring and controlling the operation of each.

In an exemplary embodiment, the system 3600 implements a method 3800 of diagnosing and treating a patient in which, in 3802, the system determines if a lesion has been detected by the CT scanner 3602.

If the system 3600 determines that the CT scanner 3602 has detected a lesion, then, in 3804, the system determines if the size of the lesion is greater than or equal to 1.5 cm in diameter or less than 1.5 cm in diameter. If the system 3600, in 3804, determines that the lesion is greater than or equal to 1.5 cm in diameter, then a normal treatment procedure is performed on the patient.

Alternatively, if the system 3600, in 3804, determines that the lesion is less than 1.5 cm in diameter, then a contrast agent is injected into the patient in a conventional manner 3808. In an exemplary embodiment, the contrast agent is an IntegriSense 680 fluorescent contrast agent used to label αᵥβ₃ integrin expressed in malignant cancer cells.

In an exemplary embodiment, in 3808, contrast enhancement agents are used to label tumor regions. These contrast enhancement agents include the FDA-approved ICG, as well as molecular imaging dyes targeting specific pathways. In an exemplary embodiment, in 3808, integrin dye can be used to label lung cancer tissue and may be detected using fiber-optic microendoscopy using one or more of the methods of the present exemplary embodiments. In an exemplary embodiment, in 3808, using other molecular imaging contrast agents for targeting specific molecular pathways, the fiber-optic molecular imaging guided tumor detection and diagnosis methods of the present exemplary embodiments may provide more diagnostic power and could potentially eliminate the need for a biopsy.

In an exemplary embodiment, the system 3600 then, in 3810 provides an image guided diagnosis, by operating the RF introducer needle 3606 and the microendoscopy imaging system 3608 to provide an image guide diagnosis. In an exemplary embodiment, the system 3608 provides an image guide diagnosis by processing the CARS signals generated by operation of the system to provide specific spectral features of the molecules scanned thereby to identify the lesion as being malignant cancer and/or differentiate the lesion from known healthy tissue as a proxy for malignant cancer. The specific spectral features and spectral differentiator metric associated with a known malignant lesion by itself and/or versus healthy normal tissue may be developed through analyzing empirical clinical data.

If the microendoscopy imaging system 3608 determines that the lesion is malignant cancer in 3812, then, in 3814, the RF ablation system 3610 is operated to ablate the lesion. Alternatively, if the microendoscopy imaging system 3608 does not determine that the lesion is malignant cancer in 3812, then, in 3816, the system 3600 continues in 3810 using image guided diagnosis.

In an exemplary embodiment, during operation, as illustrated in FIGS. 39, 39a, 28b and 28c, the system 3600 implements a method 3900 of operation in which, in 3902, the system provides a pre-procedural CT scan 3902a of the patient.

In an exemplary embodiment, the system 3600 then, in 3904, provides segmentation of the pre-procedural CT scans to provide better visualization of anatomical features such as, for example, pulmonary vessels, airways, as well as nodules to aid in surgical planning. In an exemplary embodiment, the segmentation of 3904 may be conventional and/or include one or more aspects of the exemplary embodiments herein.

In an exemplary embodiment, in 3904, the system 3600 performs region-growing-based lung field segmentation and vascular-oriented blood vessel segmentation on the CT images. In an exemplary embodiment, the lung field segmentation extracts cavity and lung fields, and the vessel segmentation matches vascular structures from medical images.

In 3906, the system 3600 then provides registered CT images 3906a of the patient and fuses the patient data, which may include CT scans as well as other visual and diagnostic data, into a common and user interactive database.

In an exemplary embodiment, in 3906, the system 3600 provides deformable registration of lung parenchyma: A joint segmentation and registration algorithm aligns serial images of the same patient. No particular assumption regarding the temporal pathological changes is used in the longitudinal deformation model. In an exemplary embodiment, the registration of 3906 may be conventional and/or include one or more aspects of the exemplary embodiments herein.

In 3908, the system 3600 then provides real time visualization, via one or more user interfaces 3908a, of the operational steps of the method 3900. In this manner, the method 3900 provides: a) an image guided pre-procedural image database to facilitate procedure diagnosis and planning; and b) during the treatment process, provides a real-time visualization of the operational steps of the method 3900. Furthermore, in 3908, the system 3600 may detect the interventional probe by EM tracking and visualize via CT imaging in real time. Motion compensation models, such as the exemplary embodiments of the motion compensation methods herein, may be used for simulation of real-time CT images during intervention based on the signals from respiratory belting and EM-tracking of fiducial markers. After successfully targeting the lesion, fiber-optic microendoscopy imaging is then used for further confirmation to verify that the probe tip is inside the tumor of interest through microendoscopy and fine needle aspiration biopsy (“FNAB”) is then performed to diagnose malignant tumor. Radio frequency ablation (“RFA”) treatment can be applied onsite for treatment.

In an exemplary embodiment, in 3908, the system 3600 provides an oblique view interface, including automatic alignment between the physicians orientation and standing position and the display of the images, so that physicians can easily steer the interventional probe, including one or more of the needle 3606, the fiber probe system 126, and the fiber probe 402, by referring to the image visualization during intervention.

In an exemplary embodiment, as illustrated in FIG. 39d, in one or more of 3902, 3904, 3906 and 3908, the system 3600 may implement a respiratory motion correction method, that may also include one or more aspects of the method 1900, that utilizes longitudinal 4DCT from a number of samples to construct the respiratory patterns, and correlates this pattern with the specific CT images and the real time tracking data from a patient such as fiducials attached on the patient's chest as well as the respiratory belt signals. CT images during intervention are then estimated based on the correlation model and the real time signals.

In exemplary experimental embodiments of the system 3600 and methods 3800 and 3900, lung tumor models were created using females white New Zealand rabbits. The rabbits had weights of 2.2 kg±200 g. The initial VX2 tumor cell line was provided by the Department of Comparative Medicine at M.D. Anderson. In order to propagate the VX2 tumor cell line, a tumor cell suspension was first inoculated in the limb of one rabbit and a tumor with a diameter of around 20 mm was noticeable after two weeks. From this tumor two cell suspensions were prepared, one for limb inoculation and the other one for lung inoculation. For this procedure, the rabbits were anesthetized with general anesthesia and the hair of the thoracic was shaved completely. The lung inoculation was performed under fluoroscopy guidance. Once a region of the lung of a rabbit was selected, an 18G Chiba needle was introduced into the chest of the rabbit. In order to simulate a peripheral lung tumor, the needle was placed at the base of the right lung of the rabbit and the depth of the needle was continuously assessed with different fluoroscopy views of the C-arm at 0, 45 and 90 degrees. Once the needle was in the desired location and at an adequate depth, the VX2 cell suspension was injected into the rabbit. Five minutes later new fluoroscopic images of the chest of the rabbit were taken for pneumothorax assessment. No rabbit developed pneumothorax in our experiments. The VX2 tumor size was assessed with weekly CT until a desired size of ˜15 mm was attained.

On the day of the operation of the exemplary experimental embodiments of the system 3600 and methods 3800 and 3900, the rabbit was anesthetized with general anesthesia and taken to the CT facility. A pre-procedural CT scan with breath holding was performed using the CT scanner 3602. The CT data was then transferred to the work station 3612 of the system 3600. The pre-procedural CT scan was used for image segmentation, tumor identification and surgical planning. After placing five or six active fiducials near the chest of rabbit, the coordinates of the fiducials in the EM-tracking space and the CT image space were registered using affine transformation by the work station 3612. During intervention, real-time tracking data, including the location and orientation of the intervention devices, were precisely measured by EM sensors of the EM tracking system 3604 and mapped onto the CT image space in real-time.

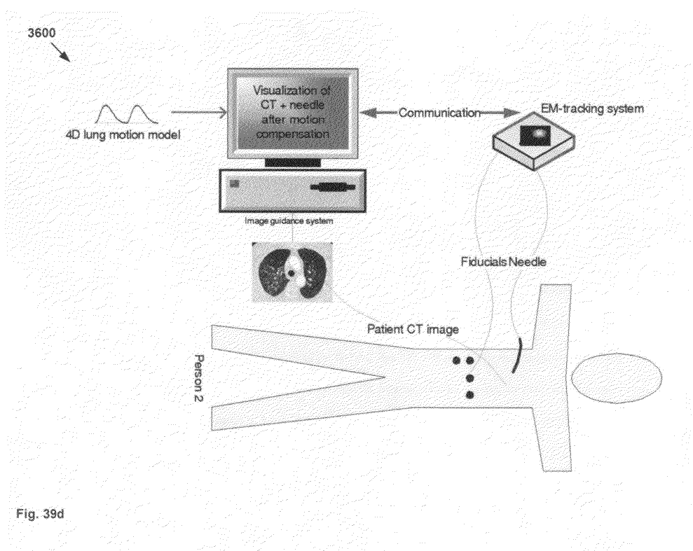

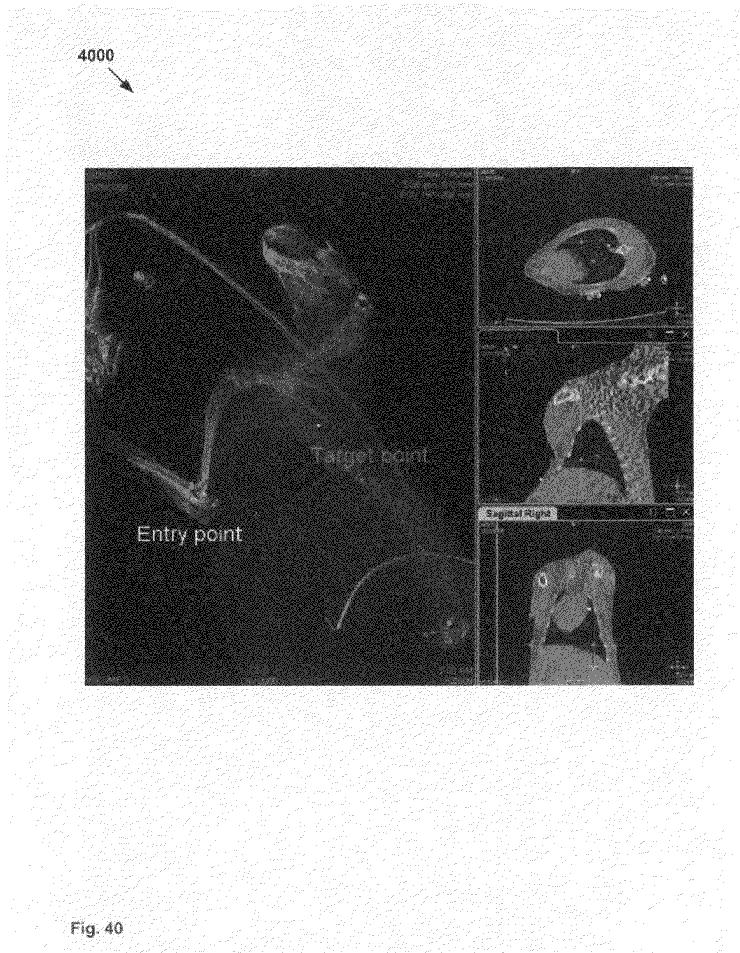

As illustrated in FIG. 40, during the exemplary experimental embodiments of the system 3600 and methods 3800 and 3900, using the CT scanner 3602, a user interface 4000 was provided on the work station 3612 that indicated a target point was selected as the center of the tumor and the entry point indicated the location for puncturing the needle. For the surgical planning of the method 3800, the pre-procedural CT volume was segmented using lung volume segmentation, as described herein, and the lung lesions were labeled and visualized in both volumetric and surface meshes. The surgical planning interface 4000 further provided the operator of the work station 3612 an interactive method for creating a path for the needle insertion. The orthogonal viewer 4000 displayed on the work station 3612, including a simultaneous axial, sagittal, and coronal view, provided a clear perspective about the depth and direction of the needle was used for reaching the tumor. Before puncturing the needle, we confirmed the needle tracking accuracy and location at the point of entry and ensured the line traced between the point of entry, skin, and the target, tumor, was planned according to the orthogonal viewer. A small 3 mm incision was made at the skin level for the needle. Before the puncture, the breathing of the animal was held thereby avoiding any movement from the chest.

As illustrated in FIGS. 40 and 41, during the exemplary experimental embodiments of the system 3600 and methods 3800 and 3900, user interfaces, 4000 and 4100, were provided on the work station 3612 that indicated that the needle tip had reached the target tumor. Once the tumor target was reached, we proceeded to needle fixation, preventing movement due to the CT gantry of respiratory movements that might displace the needle from the target location. A post-procedural scan with breath holding was performed to verify the needle location.

In the exemplary experimental embodiments of the system 3600 and methods 3800 and 3900, in order to evaluate the accuracy of the needle intervention, we calculated the distance between the manually selected needled tip from the confirmation CT and the target point obtained during surgical planning. Since the rabbit might have moved from the pre procedural to post-procedural scan, a global image registration was performed afterwards.

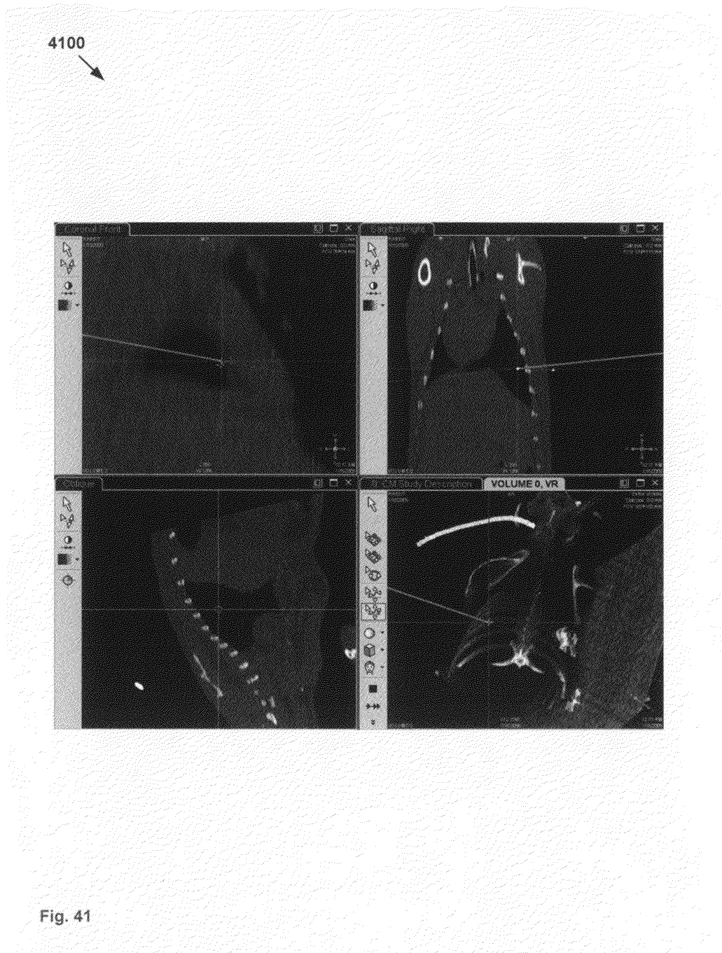

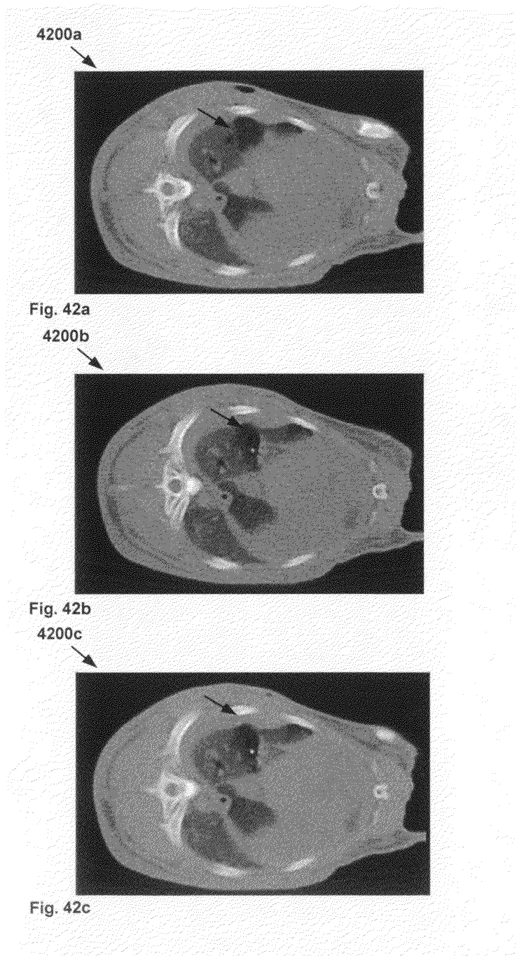

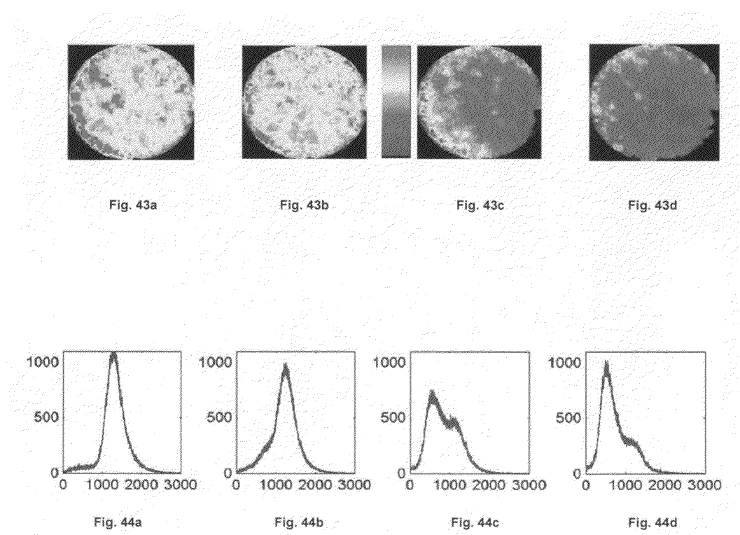

In the exemplary experimental embodiments of the system 3600 and methods 3800 and 3900, as illustrated in FIG. 42a; a user interface 4200a provided on the work station 3612 showed a screen cut of the tumor and the arrow pointed to the target point in the pre-procedural CT.

In the exemplary experimental embodiments of the system 3600 and methods 3800 and 3900, as illustrated in FIG. 42b, a user interface 4200b provided on the work station 3612 showed the corresponding slice and the arrow pointed to the needle tip in the confirmation CT.

In the exemplary experimental embodiments of the system 3600 and methods 3800 and 3900, as illustrated in FIG. 42c, a user interface 4200c provided on the work station 3612 showed the registered image. After completing these operations, the distance between the target point and the actual need tip was calculated. The result was then translated as the needle puncture accuracy.

In the exemplary experimental embodiments of the system 3600 and methods 3800 and 3900, the experiments were conducted on eight rabbits and they were numbered in the experiment time sequence. The first three experiments were performed without breath holding while the later five were breath holding, by removing the ventilator, cases. The distance between the target point in the pre-CT and the needle tip location in the confirmation CT, after global image registration using the conventional FSL FLIRT program, was used as a metric for the accuracy. Table 5 below lists the results. Overall the average distance without breath holding was 11 mm, and the average error for the later five experiments with breath holding was 3.5 mm which meant that the lung movement caused by breathing impacted the accuracy of intervention. After using the breath holding procedure, the accuracy of puncturing was improved up to 70%.

TABLE 5
Accuracy of intervention experiments

| Rabbit | Resolution (mm) | Accuracy (mm) |
|---|---|---|
| 1 | (0.27, 0.27, 1.20) | 12.45 |
| 2 | (0.26, 0.26, 1.20) | 10.68 |
| 3 | (0.29, 0.29, 1.20) | 10.32 |
| 4 | (0.28, 0.28, 1.20) | 2.93 |
| 5 | (0.23, 0.23, 1.20) | 4.90 |
| 6 | (0.28, 0.28, 1.20) | 4.84 |
| 7 | (0.28, 0.28, 1.20) | 1.25 |
| 8 | (0.25, 0.25, 1.20) | 3.67 |

In the exemplary experimental embodiments of the system 3600 and methods 3800 and 3900, in order to validate the effectiveness of the microendoscopic procedure, we collected thirty microendoscopy video clips from the six rabbits' lung tumors to test the precision of the system 3600 and methods 3800 and 3900. As illustrated in FIGS. 43a, 43b, 43c and 43d, fluorescence microendoscopy images were captured during the rabbit experiments. The motion of the image sequences, 43a, 43b, 43c and 43d, were corrected and the intensities were color-coded for better visualization. The histograms, 44a, 44b, 44c and 44d, of the images, 43a, 43b, 43c and 43d, respectively, within a one-second sliding window at each timepoint were also generated. The images, 43a and 43b, showed molecular imaging results within the tumor and the images, 43c and 43d, showed molecular imaging results at the boundary of the tumor. The histograms, 44a, 44b, 44c and 44d, illustrate the intensity distributions of the images within temporal sliding windows that were different within and outside the tumor and there was a significantly different contrast or distribution difference between images with non-labeled tissue and αᵥβ₃-labeled tissue. The histograms, 44a, 44b, 44c and 44d, had peak values of non-labeled tissue images at around 500 and peak values of αᵥβ₃-labeled tissue images at around 1500. Thus, the histograms were classified into two different groups with a threshold value of 1000 providing a metric that indicated a malignant tumor. These exemplary experimental results for the system 3600 and methods 3800 and 3900 were unexpected results.

As demonstrated by the exemplary experimental embodiments of the system 3600 and methods 3800 and 3900, the system and methods provided a convenient, efficient and accurate interventional tool for on-the-spot diagnosis and treatment of malignant tumors. Furthermore, as validated by the exemplary experimental results using a predictive animal model, the system 3600 and methods 3800 and 3900 permitted a fine needle aspiration biopsy that can be used to diagnose cancer in humans, where the RF ablation can also be applied for confirmed malignant cancerous cases. In addition, as demonstrated by the exemplary experimental results using a predictive animal model, the system 3600 and methods 3800 and 3900 is particularly effective for lung tumor intervention and molecular imaging diagnosis. After successfully targeting the tumor, the molecular imaging of the system 3600 and methods 3800 and 3900 can be used for onsite diagnosis which could be followed by RF ablation treatment. Furthermore, as demonstrated by the exemplary experimental results using a predictive animal model, the system 3600 and methods 3800 and 3900, the system and method permits the design and planning of the needle trajectory for puncture using an orthogonal viewer, with simultaneous axial, sagittal, and coronal view, which provides a clear perspective regarding the depth and the direction of the needle for reaching the tumor.

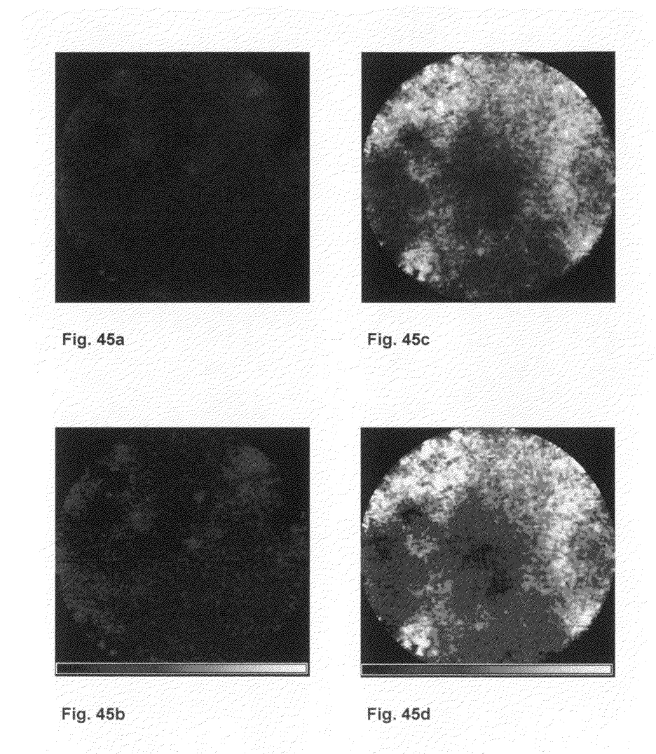

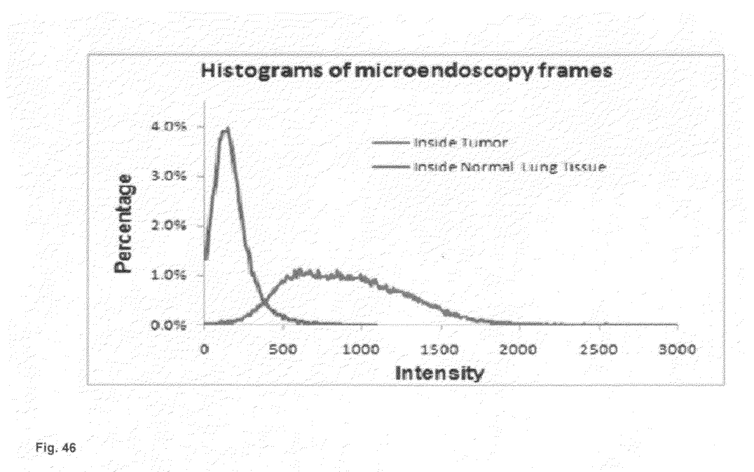

Referring now to FIGS. 45a, 45b, 45c and 45d, in exemplary experimental embodiments of the system 3600 and methods 3800 and 3900, normal lung tissue and a malignant tumor within the lung tissue were differentiated from one another permitting a highly accurate diagnosis of a malignant lung tumor. In particular, normal lung tissue, FIGS. 45a and 45b, and malignant lung tumor tissue, FIGS. 45c and 45d, were differentiated from one another. Furthermore, histograms, FIG. 46, of the images obtained during exemplary experimental embodiments of the system 3600 and methods 3800 and 3900, showed a clear and distinct difference between the normal lung tissue and the malignant lung tumor tissue. This was an unexpected result.

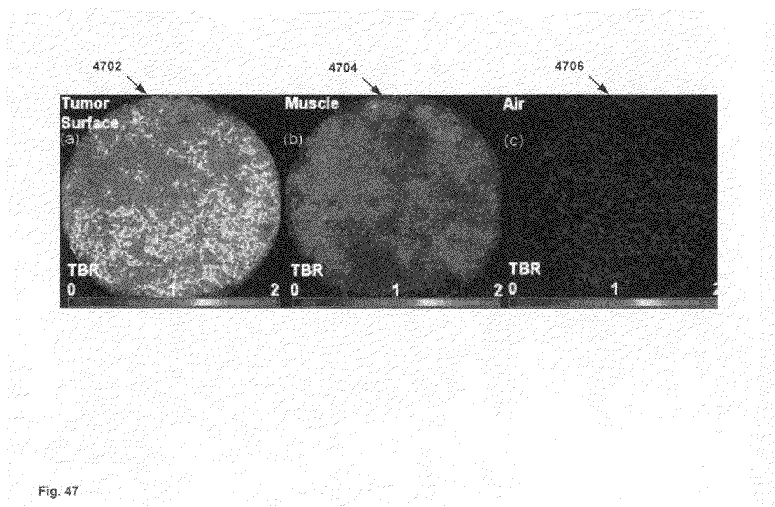

Referring now to FIG. 47, in exemplary experimental embodiments of the system 3600 and methods 3800 and 3900, fluorescent microendoscopic images of αᵥβ₃ expressing cells (TBR>1.5) were obtained that included an image 4702 on the tumor surface, an image 4704 on a muscle, and an image 4706 in air. As demonstrated by the exemplary images in FIG. 47, the images obtained during exemplary experimental embodiments of the system 3600 and methods 3800 and 3900 showed a clear and distinct difference between the malignant tumor, muscle, and air. This was an unexpected result.

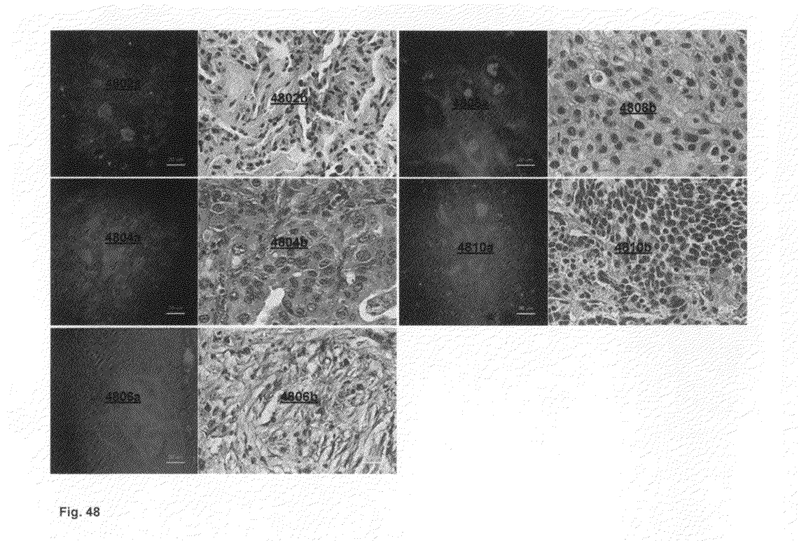

Referring to now to FIGS. 48, in exemplary experimental embodiments of the system 3600 and methods 3800 and 3900, a CARS image 4802a of a normal lung cell, a CARS image 4804a of an Adenocarcinoma lung cancer cell, a CARS image 4806a of an organizing pneumonia cell, a CARS image 4808a of a squamos lung cancer cell and a CARS image 4810a of a small cell lung cancer were obtained. The CARS images obtained, 4802a, 4804a, 4806a, 4808a and 4810a, were also compared with photomicroscopic images of the corresponding cells, 4802b, 4804b, 4806b, 4808b and 4810b, to confirm the differentiation of the cells provided by the system 3600 and methods 3800 and 3900. As demonstrated by the exemplary images in FIG. 48, the images obtained during exemplary experimental embodiments of the system 3600 and methods 3800 and 3900, showed clear and distinct differences between and among the various cell types thereby permitting accurate diagnosis. This was an unexpected result.

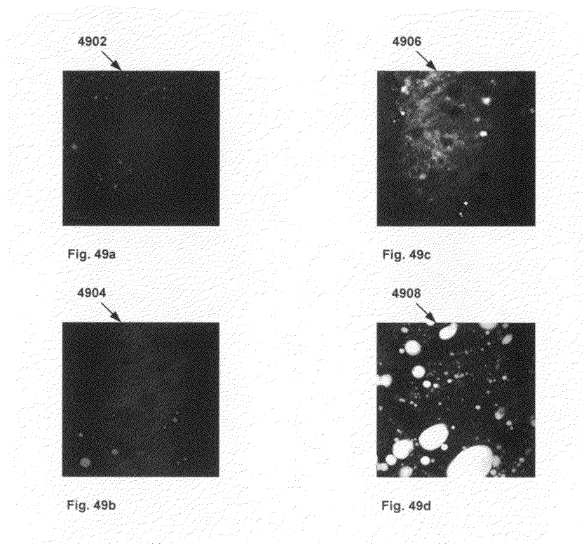

Referring to now to FIGS. 49a, 49b, 49c and 49d, in exemplary experimental embodiments of the system 3600 and methods 3800 and 3900, a CARS image 4902 of a normal beast tissue, a CARS image 4904 of normal breast tissue, a CARS image 4906 of a breast tumor and a CARS image 4910 of a breast tumor were obtained. As demonstrated by the exemplary images in FIGS. 49a, 49b, 49c and 49d, the images obtained during exemplary experimental embodiments of the system 3600 and methods 3800 and 3900, showed clear and distinct differences between and among the various cell types thereby permitting accurate diagnosis of malignant breast cancer. This was an unexpected result.

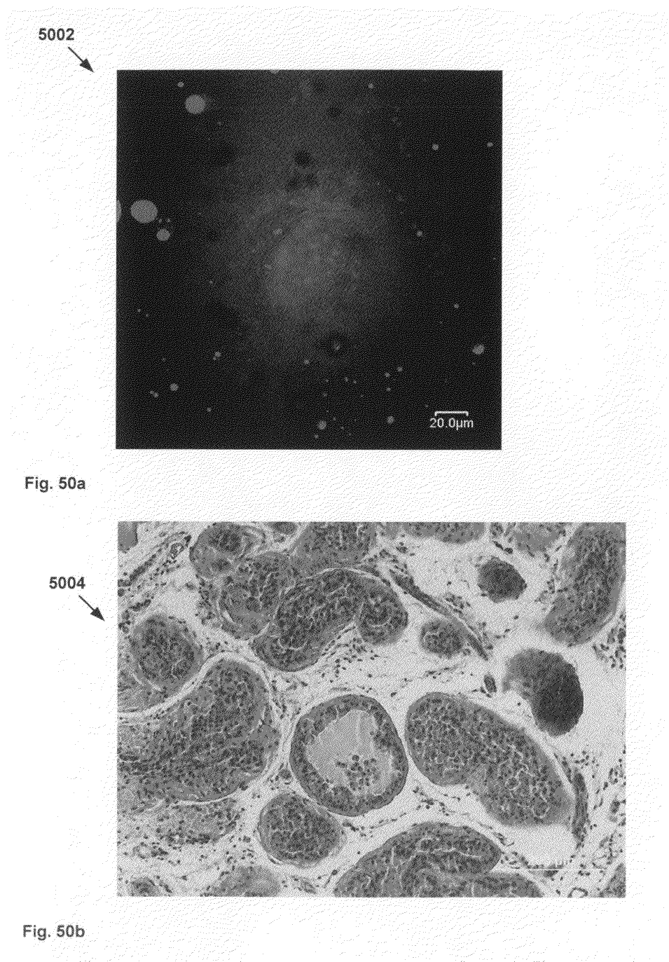

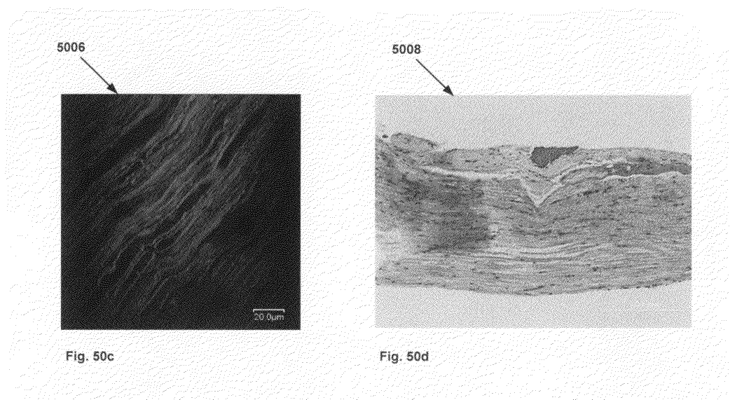

Referring to now to FIGS. 50a and 50b, in exemplary experimental embodiments of the system 3600 and methods 3800 and 3900, a CARS image 5002 of a mouse dorsal prostate was obtained and compared with a photomicroscopic image of the corresponding cells 5004. The CARS images of the mouse dorsal prostate may be differentiated using CARS images. Thus, the exemplary experimental embodiments of the system 3600 and methods 3800 and 3900, demonstrated that prostate cells may be easily differentiated from non-prostate cells during treatment and surgery. This was an unexpected result.

Referring to now to FIGS. 50c and 50d, in exemplary experimental embodiments of the system 3600 and methods 3800 and 3900, a CARS image 5006 of the myelin sheath of a mouse body nerve was obtained and compared with a photomicroscopic image of the corresponding cells 5008. The CARS images of nerve sheath cells may be differentiated using CARS images since such cells are rich in CH2 chemical bonds which may be easily recognized using CARS images. Thus, the exemplary experimental embodiments of the system 3600 and methods 3800 and 3900, demonstrated that nerve cells may be easily differentiated from non-nerve cells during treatment and surgery. This was an unexpected result. This will permit surgeons to avoid cutting into nerve cells during surgery which is particularly prevelant in conventional prostate surgery. Thus, the system 3600 and methods 3800 and 3900 may be used in surgery to minimize damage to nerves.

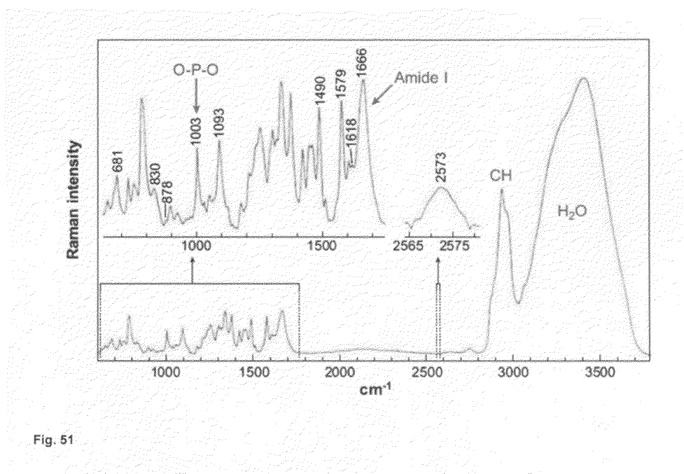

In an exemplary embodiment of the system 3600 and methods 3800 and 3900, specific diseases may be identified based upon the unique attributes of the vibrational frequencies of the chemical bonds: 1) the peak positions which provide a unique chemical fingerprint for each molecule; and/or 2) the peak intensities, which provide concentration information. In particular, as illustrated in FIG. 51, in exemplary experimental embodiments of the system 3600 and methods 3800 and 3900, specific diseases such as, for example, the P22 virus were identified. This was an unexpected result.

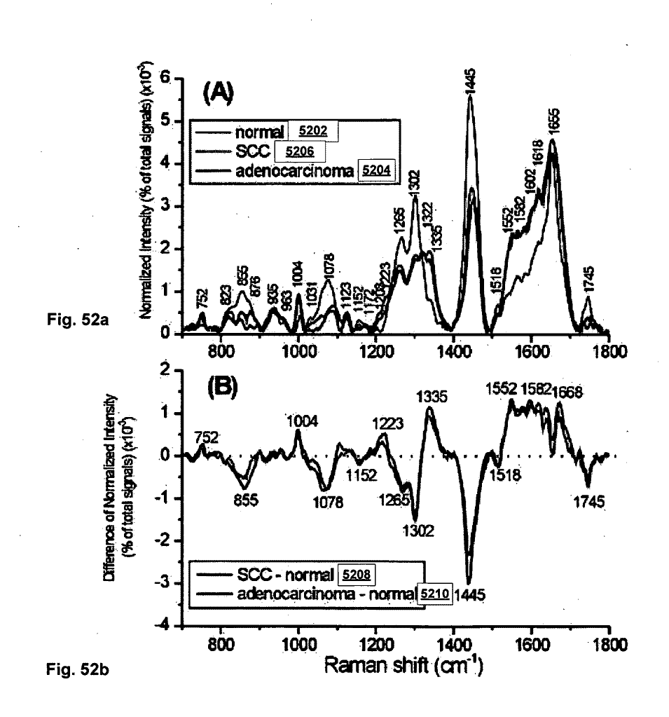

In an exemplary embodiment of the system 3600 and methods 3800 and 3900, lung cancer may be differentiated from normal lung tissue based upon the unique attributes of the vibrational frequencies of the chemical bonds: 1) the peak positions which provide a unique chemical fingerprint for each molecule; and/or 2) the peak intensities, which provide concentration information. In particular, as illustrated in FIG. 52a, in exemplary experimental embodiments of the system 3600 and methods 3800 and 3900, Raman spectra 5202 for normal bronchial tissue, Raman spectra 5204 for malignant adenocarcinoma, and Raman spectra 5206 for skin squamous cell carcinoma (“SCC”) were obtained. Each of the spectra, 5202, 5204 and 5206, were normalized to the integrated area under each spectra to correct for variations in the absolute spectral intensity. Furthermore, as illustrated in FIG. 52b, difference spectra, 5208 and 5210, for the SCC and the adenocarcinoma, respectively, were then obtained by subtracting the spectra 5202 from the spectra 5206 and 5204, respectively. As illustrated in FIG. 52b, the use of the difference spectra, 5208 and 5210, provides a graphical means for differentiating normal lung tissue from cancerous lung tissue. Furthermore, the difference spectra, 5208 and 5210, further provides a graphical means for differentiating different types of lung cancer. This was an unexpected result.

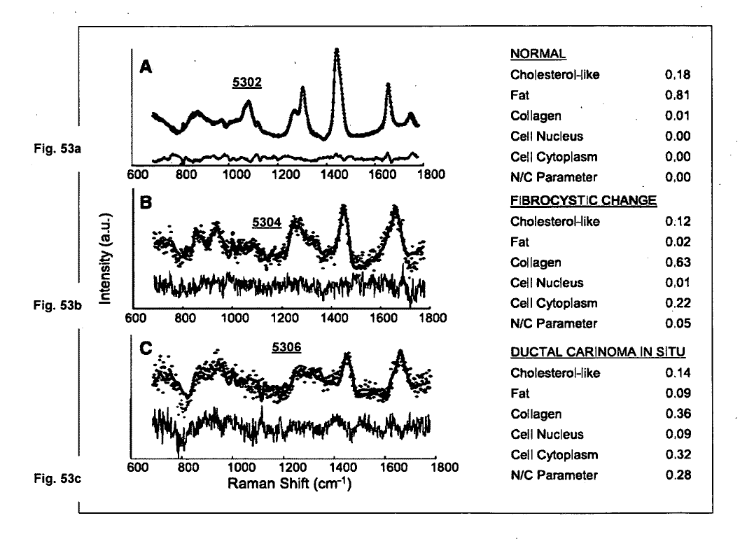

In an exemplary embodiment of the system 3600 and methods 3800 and 3900, breast cancer may be differentiated from normal breast tissue based upon the unique attributes of the vibrational frequencies of the chemical bonds: 1) the peak positions which provide a unique chemical fingerprint for each molecule; and/or 2) the peak intensities, which provide concentration information. In particular, as illustrated in FIGS. 53a, 53b and 53c, in exemplary experimental embodiments of the system 3600 and methods 3800 and 3900, Raman spectra 5302 for normal breast tissue, Raman spectra 5304 for fibrocystic breast tissue, and Raman spectra 5306 for ductal carcinoma breast were obtained. As illustrated in FIGS. 53a, 53b and 53c, the spectra obtained provides a graphical means for differentiating between different types of breast tissue. This was an unexpected result.

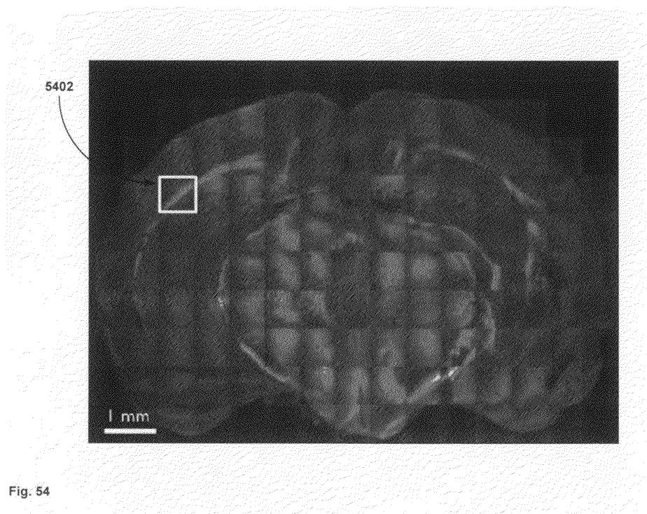

In an exemplary embodiment of the system 3600 and methods 3800 and 3900, brain tissues may be differentiated from one another during surgery to permit more precise and safer brain surgery. In particular, as illustrated in FIG. 54, different brain tissues may be differentiated from one another during surgery to, for example, distinguish deep white matter tracts 5402 from other brain tissue. This was an unexpected result.

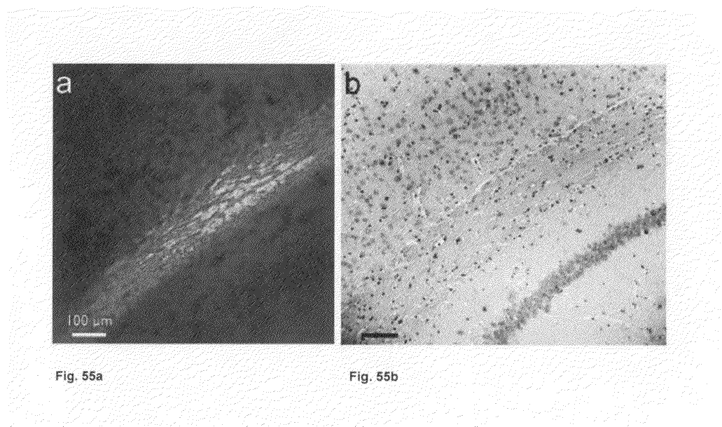

In an exemplary embodiment of the system 3600 and methods 3800 and 3900, intermediate special resolution images for gross anatomy and high resolution images may be produced from the same imaging acquisition. In particular, as illustrated in FIG. 55a, a single CARS image was obtained from a region of a mouse brain. FIG. 55b illustrates the same region of the mouse brain different brain using a Hematoxylin and Eosin stain (“H & E”). Note that both images, FIGS. 55a and 55b, provided the observable structures, from upper left corner to bottom right corner, of cortex, corpus callosum, oriens layer, and pyramidal layer. This was an unexpected result.

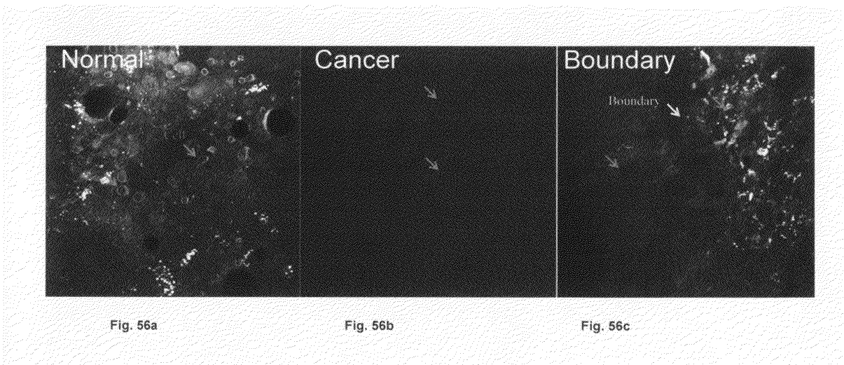

In an exemplary experimental embodiment of the system 3600 and methods 3800 and 3900, a human lung cancer cell line (A549) was used to induce lung cancer on a mouse xenograph cancer model. As illustrated in FIG. 56a, normal lung tissue from the mouse xenograph cancer model was imaged. As illustrated in FIG. 56b, cancerous lung tissue from the mouse xenograph cancer model was imaged. As illustrated in FIG. 56c, the margin between normal and cancerous lung tissue from the mouse xenograph cancer model was imaged. In the exemplary experimental embodiments, normal and cancerous lung tissues were images at 2845 cm-1 wave number, which corresponds to the excitation peak of intrinsic lipid molecules. In the exemplary experimental embodiments, the cancerous lung tissue provides a CARS signal indicating a lower lipid level, which can be used for differential diagnosis. This was an unexpected result.

In an exemplary embodiment, during operation of the system 3600 and methods 3800 and 3900, intermediate special resolution images for gross anatomy and high resolution images may be produced from the same imaging acquisition. In particular, as illustrated in FIG. 55a, a single CARS image was obtained from a region of a mouse brain. FIG. 55b illustrates the same region of the mouse brain different brain using a Hematoxylin and Eosin stain (“H & E”). Note that both images, FIGS. 55a and 55b, provide the observable structures, from upper left corner to bottom right corner, of cortex, corpus callosum, oriens layer, and pyramidal layer. This was an unexpected result.

The present exemplary embodiments provide systems and methods for: 1) Improving the accuracy and efficiency for intervention procedures such as reduced procedure time and number of repetitive scans needed with more accurate localization; 2) improved diagnostic yield for fine needle aspiration biopsy, with the fiber-optic imaging guided tumor detection technique; 3) on-the-spot diagnosis and treatment care; and 4) a percutaneous interventional guidance system for on-the-spot and onsite diagnosis and treatment for small cell lung cancer, particularly for peripheral lung cancer at it early stage.

Furthermore, the exemplary embodiments improve upon the deficiencies associated with convention treatment in which typical lung cancer diagnoses are only reached after four different imaging studies, both non-invasive and much more invasive, such as percutaneous biopsies and subsequent transthoracic CT-guided needle biopsies. These studies typically take place over the course of days or weeks, and are then followed by treatment. By contrast, the present exemplary embodiment, for example, provide a user-friendly 3D visualization and navigation platform that allows physicians to quickly and accurately guide a needle to the small nodules of potential cancer in patients' lungs. In the exemplary embodiments, once in the nodule, the physician may use molecular imaging to get a viable tissue sample through a fine-needle aspiration biopsy. Then, in the exemplary embodiments, if cancer is detected, the physician may use RF ablation to treat the cancer immediately on the spot.

An endoscopic microscopy apparatus has been described that includes an optical fiber; a collimating lens set operably coupled to one end of the optical fiber; a scanning mirror operably coupled to the optical fiber proximate the collimating lens; an objective lens set operably coupled to the optical fiber; a coupling lens operably coupled to another end of the optical fiber; an optical coupling assembly operably coupled to the coupling lens; a data acquisition system operably coupled to the optical coupling assembly; and a source of a plurality of laser beams operably coupled to the optical coupling assembly. In an exemplary embodiment, the apparatus further includes an optical time delay operably coupled between the source of laser beams and the optical coupling assembly adapted to controllably delaying transmission of one of the laser beams. In an exemplary embodiment, the optical coupling assembly comprises one or more wave division multiplexers. In an exemplary embodiment, the optical coupling assembly comprises a plurality of wave division multiplexers. In an exemplary embodiment, the optical coupling assembly comprises a plurality of wave division multiplexers that are cascaded with respect to one another. In an exemplary embodiment, the apparatus further includes a motion correction system operably coupled to the data acquisition system.

A method of operating an endoscopic microscopy apparatus has been described that includes operating the apparatus to obtain an image sequence; calculating global registration for one or more of the images; applying the global registration to one or more of the images; calculating deformable registration for one or more of the images; and applying the deformable registration to one or more of the images. In an exemplary embodiment, calculating the global registration for one or more of the images comprises calculating the global registration for one or more of the images by iteratively minimizing an energy equation that is a function of normalized mutual information. In an exemplary embodiment, the energy function comprises a linear portion and a non-linear portion. In an exemplary embodiment, calculating the global registration for one or more of the images by iteratively minimizing an energy equation that is a function of normalized mutual information comprises iteratively estimating an actual transformation one or more of the images. In an exemplary embodiment, calculating the global registration for one or more of the images by iteratively minimizing an energy equation that is a function of normalized mutual information comprises iteratively estimating an actual transformation one or more of the images; and optimizing the estimate of the actual transformation. In an exemplary embodiment, calculating the global registration for one or more of the images by iteratively minimizing an energy equation that is a function of normalized mutual information comprises iteratively estimating an actual transformation one or more of the images; and optimizing the estimate of the actual transformation using cubature kalman filtering. In an exemplary embodiment, calculating the global registration for one or more of the images comprises estimating motion within one or more of the images using line by line searching; dividing one or more of the images into resting and movement time periods; and using a speed embedded hidden markov model for motion correction of one or more of the images. In an exemplary embodiment, calculating the global registration for one or more of the images comprises using a speed embedded hidden markov model for motion correction of one or more of the images. In an exemplary embodiment, using a speed embedded hidden markov model for motion correction of one or more of the images comprises defining a state observation probability and a state transition probability. In an exemplary embodiment, using a speed embedded hidden markov model for motion correction of one or more of the images comprises defining a state observation probability and a state transition probability; and maximizing a value of a function of the state observation probability and the state transition probability. In an exemplary embodiment, using a speed embedded hidden markov model for motion correction of one or more of the images comprises defining a state observation probability and a state transition probability; maximizing a value of a function of the state observation probability and the state transition probability; and determining a most likely sequence of image offsets. In an exemplary embodiment, using a speed embedded hidden markov model for motion correction of one or more of the images comprises defining a state observation probability and a state transition probability; maximizing a value of a function of the state observation probability and the state transition probability; determining a most likely sequence of image offsets; and determining an optimal image offset sequence. In an exemplary embodiment, calculating the global registration for one or more of the images comprises preprocessing one or more of the images; training a motion estimating model for one or more of the images; and estimating a motion correction model for one or more of the images.

It is understood that variations may be made in the above without departing from the scope of the invention. While specific embodiments have been shown and described, modifications can be made by one skilled in the art without departing from the spirit or teaching of this invention. The embodiments as described are exemplary only and are not limiting. Many variations and modifications are possible and are within the scope of the invention. Furthermore, one or more elements of the exemplary embodiments may be omitted, combined with, or substituted for, in whole or in part, one or more elements of one or more of the other exemplary embodiments. Accordingly, the scope of protection is not limited to the embodiments described, but is only limited by the claims that follow, the scope of which shall include all equivalents of the subject matter of the claims.

1. A system for diagnosis and treatment, comprising:
a microendoscopic imaging system;
a CT scanner;
a RF ablation system;
an RF introducer needle;
an EM tracking system; and
a computer work station operably coupled to the microendoscopic imaging system; the CT scanner; the RF ablation system; the RF introducer needle; and the EM tracking system for monitoring and controlling the operation one or more of the microendoscopic imaging system; the CT scanner; the RF ablation system; the RF introducer needle; and the EM tracking system.

2. The system of claim 1, wherein the microendoscopic imaging system comprises:
an optical fiber;
a collimating lens set operably coupled to the optical fiber;
a scanning mirror operably coupled to the optical fiber; and
an objective lens set operably coupled to the optical fiber.

3. The system of claim 2, wherein the optical fiber comprises a single mode fiber.

4. The system of claim 2, wherein the optical fiber comprises a multimode fiber.

5. The system of claim 2, wherein the optical fiber comprises a single mode fiber and a multimode fiber.

6. The system of claim 2, further comprising a controller operably coupled to the scanning mirror; wherein the controller is adapted to actuate the mirror.

7. The system of claim 2, wherein the controller is adapted to displace the mirror in a 2-D scanning pattern.

8. The system of claim 5, wherein the controller is adapted to displace the mirror in a raster scanning pattern.

9. The system of claim 2, wherein the optical fiber comprises a double clad photonic crystal fiber.

10. The system of claim 2, wherein the objective lens set provides a viewing resolution greater than or equal to 1124 nm.

11. The system of claim 2, wherein the objective lens set provides a laser spot size of greater than or equal to 2248 nm.

12. The system of claim 2, wherein the scanning mirror has a maximal scanning angle of up to 2.784 degrees.

13. The system of claim 2, wherein the objective lens set provides an image having up to 237×237 pixels.

14. The system of claim 2, wherein the scanning mirror has a linear scan step greater than or equal to about 21 μm.

15. The system of claim 2, wherein the maximal signal to noise ratio is less than or equal to about 3.5.

16. The system of claim 1, wherein the microendoscopic imaging system comprises:
an optical fiber;
a collimating lens set operably coupled to one end of the optical fiber;
a scanning mirror operably coupled to the optical fiber proximate the collimating lens;
an objective lens set operably coupled to the optical fiber;
a coupling lens operably coupled to another end of the optical fiber;
an optical coupling assembly operably coupled to the coupling lens;
a data acquisition system operably coupled to the optical coupling assembly; and
a source of a plurality of laser beams operably coupled to the optical coupling assembly.

17. The system of claim 16, further comprising:
an optical time delay operably coupled between the source of laser beams and the optical coupling assembly adapted to controllably delaying transmission of one of the laser beams.

18. The system of claim 16 or 17, wherein the optical coupling assembly comprises one or more wavelength division multiplexers.

19. The system of claim 18, wherein the optical coupling assembly comprises a plurality of wavelength division multiplexers.

20. The system of claim 19, wherein the optical coupling assembly comprises a plurality of wavelength division multiplexers that are cascaded with respect to one another.

21. The system of claim 16, further comprising:
a motion correction system operably coupled to the data acquisition system.

22. The system of claim 16, wherein the optical fiber comprises a single mode fiber.

23. The system of claim 16, wherein the optical fiber comprises a multimode fiber.

24. The system of claim 16, wherein the optical fiber comprises a single mode fiber and a multimode fiber.

25. The system of claim 16, wherein the optical fiber comprises a double clad photonic crystal fiber.

26. The system of claim 25, wherein a portion of the optical fiber comprises a single mode fiber; and wherein another portion of the optical fiber comprises a multimode fiber.

27. The system of claim 16, wherein the objective lens set provides a viewing resolution greater than or equal to 1124 nm.

28. The system of claim 16, wherein the objective lens set provides a laser spot size of greater than or equal to 2248 nm.

29. The system of claim 16, wherein the scanning mirror has a maximal scanning angle of up to 2.784 degrees.

30. The system of claim 16, wherein the objective lens set provides an image having up to 237×237 pixels.

31. The system of claim 16, wherein the scanning mirror has a linear scan step greater than or equal to about 21 μm.

32. The system of claim 16, wherein the maximal signal to noise ratio is less than or equal to about 3.5.

33. The system of claim 1, wherein the microendoscopic imaging system comprises:
a source of a stokes laser beam;
a source of a pump laser beam;
an optical fiber operably coupled to the sources of the stokes and pump laser beams for conveying the stokes and pump laser beams;
a long pass filter operably coupled to the optical fiber; and
one or more optical detectors operably coupled to the optical fiber for detecting CARS signals;
wherein the optical fiber comprises a multimode fiber.

34. The system of claim 33, wherein a portion of the optical fiber comprises a single mode fiber.

35. The system of claim 33, wherein the optical fiber comprises a multimode fiber.

36. The system of claim 33, wherein the optical fiber comprises a single mode fiber and a multimode fiber.

37. The system of claim 33, wherein the system provides a viewing resolution greater than or equal to 1124 nm.

38. The system of claim 33, wherein the system provides a laser spot size of greater than or equal to 2248 nm.

39. The system of claim 33, wherein the system has a maximal scanning angle of up to 2.784 degrees.

40. The system of claim 33, wherein the system provides an image having up to 237×237 pixels.

41. The system of claim 33, wherein the system has a linear scan step greater than or equal to about 21 μm.

42. The system of claim 33, wherein the maximal signal to noise ratio is less than or equal to about 3.5.

43. A method for diagnosing and treating a patient, comprising:
obtaining one or more CARS images of tissue within the patient; and
as a function of one or more attributes of the CARS images, determining if the tissue comprises malignant lung cancer cells.

44. The method of claim 43, further comprising:
if the CARS images indicate that the tissue comprises malignant lung cancer cells, then removing at least a portion of the malignant lung cancer cells using RF ablation.

45. The method of claim 43, further comprising:
calculating global registration for one or more of the images;
applying the global registration to one or more of the images;
calculating deformable registration for one or more of the images; and
applying the deformable registration to one or more of the images.

46. The method of claim 45, wherein calculating the global registration for one or more of the images comprises:
calculating the global registration for one or more of the images by iteratively minimizing an energy equation that is a function of normalized mutual information.

47. The method of claim 46, wherein the energy function comprises a linear portion and a non-linear portion.

48. The method of claim 46, wherein calculating the global registration for one or more of the images by iteratively minimizing an energy equation that is a function of normalized mutual information comprises:
iteratively estimating an actual transformation one or more of the images.

49. The method of claim 46, wherein calculating the global registration for one or more of the images by iteratively minimizing an energy equation that is a function of normalized mutual information comprises:
iteratively estimating an actual transformation one or more of the images; and
optimizing the estimate of the actual transformation.

50. The method of claim 46, wherein calculating the global registration for one or more of the images by iteratively minimizing an energy equation that is a function of normalized mutual information comprises:
iteratively estimating an actual transformation one or more of the images; and
optimizing the estimate of the actual transformation using cubature kalman filtering.

51. The method of claim 45, wherein calculating the global registration for one or more of the images comprises:
estimating motion within one or more of the images using line by line searching;
dividing one or more of the images into resting and movement time periods; and
using a speed embedded hidden markov model for motion correction of one or more of the images.

52. The method of claim 45, wherein calculating the global registration for one or more of the images comprises:
using a speed embedded hidden markov model for motion correction of one or more of the images.

53. The method of claim 51 or 52, wherein using a speed embedded hidden markov model for motion correction of one or more of the images comprises:
defining a state observation probability and a state transition probability.

54. The method of claim 53, wherein using a speed embedded hidden markov model for motion correction of one or more of the images comprises:
defining a state observation probability and a state transition probability; and
maximizing a value of a function of the state observation probability and the state transition probability.

55. The method of claim 54, wherein using a speed embedded hidden markov model for motion correction of one or more of the images comprises:
defining a state observation probability and a state transition probability;
maximizing a value of a function of the state observation probability and the state transition probability; and
determining a most likely sequence of image offsets.

56. The method of claim 55, wherein using a speed embedded hidden markov model for motion correction of one or more of the images comprises:
defining a state observation probability and a state transition probability;
maximizing a value of a function of the state observation probability and the state transition probability;
determining a most likely sequence of image offsets; and
determining an optimal image offset sequence.

57. The method of claim 45, wherein calculating the global registration for one or more of the images comprises:
preprocessing one or more of the images;
training a motion estimating model for one or more of the images; and
estimating a motion correction model for one or more of the images.

58. The method of claim 57, wherein preprocessing one or more of the images comprises:
segmenting one or more of the images;
serially registering one or more of the images; and
registering a first timepoint images of the images onto a template image.

59. The method of claim 57, wherein training the motion estimating model for one or more of the images comprises:
extracting normalized surface motion vectors and corresponding fiducial motion vectors for one or more of the images;
constructing a motion statistical model by performing kernel principal component analysis on the surface motion vectors; and
training the motion estimating model using least squared support vector machine to model a relationship between the fiducial motion vectors and the surface motion vectors on kernel principal component analysis space.

60. The method of claim 57, wherein estimating the motion correction model for one or more of the images:
transferring respiratory signals of a patient onto a template space in order to use the motion estimating model to estimate motion vectors and reconstruct surface motion vectors of the patient;
generating serial deformations using the surface motion vectors as constraints in a serial deformation simulator; and
transforming the serial deformations onto a subject space to generate serial images of the patient.

61. The method of claim 43, further comprising:
calculating a global registration for one or more of the images by iteratively minimizing an energy function that is a function of normalized mutual information.

62. The method of claim 61, wherein the energy function comprises a linear portion and a non-linear portion.

63. The method of claim 61, wherein calculating the global registration for one or more of the images by iteratively minimizing an energy equation that is a function of normalized mutual information comprises:
iteratively estimating an actual transformation one or more of the images.

64. The method of claim 61, wherein calculating the global registration for one or more of the images by iteratively minimizing an energy equation that is a function of normalized mutual information comprises:
iteratively estimating an actual transformation one or more of the images; and
optimizing the estimate of the actual transformation.

65. The method of claim 61, wherein calculating the global registration for one or more of the images by iteratively minimizing an energy equation that is a function of normalized mutual information comprises:
iteratively estimating an actual transformation one or more of the images; and
optimizing the estimate of the actual transformation using cubature kalman filtering.

66. The method of claim 43, further comprising:
estimating motion within one or more of the images using line by line searching;
dividing one or more of the images into resting and movement time periods; and
using a speed embedded hidden markov model for motion correction of one or more of the images.

67. The method of claim 66, wherein using a speed embedded hidden markov model for motion correction of one or more of the images comprises:
defining a state observation probability and a state transition probability.

68. The method of claim 67, wherein using a speed embedded hidden markov model for motion correction of one or more of the images comprises:
defining a state observation probability and a state transition probability; and
maximizing a value of a function of the state observation probability and the state transition probability.

69. The method of claim 68, wherein using a speed embedded hidden markov model for motion correction of one or more of the images comprises:
defining a state observation probability and a state transition probability;
maximizing a value of a function of the state observation probability and the state transition probability; and
determining a most likely sequence of image offsets.

70. The method of claim 69, wherein using a speed embedded hidden markov model for motion correction of one or more of the images comprises:
defining a state observation probability and a state transition probability;
maximizing a value of a function of the state observation probability and the state transition probability;
determining a most likely sequence of image offsets; and
determining an optimal image offset sequence.

71. The method of claim 43, further comprising:
using a speed embedded hidden markov model for motion correction of one or more of the images.

72. The method of claim 71, wherein using a speed embedded hidden markov model for motion correction of one or more of the images comprises:
defining a state observation probability and a state transition probability.

73. The method of claim 72, wherein using a speed embedded hidden markov model for motion correction of one or more of the images comprises:
defining a state observation probability and a state transition probability; and
maximizing a value of a function of the state observation probability and the state transition probability.

74. The method of claim 73, wherein using a speed embedded hidden markov model for motion correction of one or more of the images comprises:
defining a state observation probability and a state transition probability;
maximizing a value of a function of the state observation probability and the state transition probability; and
determining a most likely sequence of image offsets.

75. The method of claim 74, wherein using a speed embedded hidden markov model for motion correction of one or more of the images comprises:
defining a state observation probability and a state transition probability;
maximizing a value of a function of the state observation probability and the state transition probability;
determining a most likely sequence of image offsets; and
determining an optimal image offset sequence.

76. The method of claim 43, further comprising:
preprocessing one or more of the images;
training a motion estimating model for one or more of the images; and
estimating a motion correction model for one or more of the images.

77. The method of claim 76, wherein preprocessing one or more of the images comprises:
segmenting one or more of the images;
serially registering one or more of the images; and
registering a first timepoint images of the images onto a template image.

78. The method of claim 76, wherein training the motion estimating model for one or more of the images comprises:
extracting normalized surface motion vectors and corresponding fiducial motion vectors for one or more of the images;
constructing a motion statistical model by performing kernel principal component analysis on the surface motion vectors; and
training the motion estimating model using least squared support vector machine to model a relationship between the fiducial motion vectors and the surface motion vectors on kernel principal component analysis space.

79. The method of claim 76, wherein estimating the motion correction model for one or more of the images:
transferring respiratory signals of a patient onto a template space in order to use the motion estimating model to estimate motion vectors and reconstruct surface motion vectors of the patient;
generating serial deformations using the surface motion vectors as constraints in a serial deformation simulator; and
transforming the serial deformations onto a subject space to generate serial images of the patient.

80. A method for diagnosing and treating a patient, comprising:
obtaining one or more CARS images of tissue within the patient; and
as a function of one or more attributes of the CARS images, determining if the tissue comprises malignant breast cancer cells.

81. The method of claim 80, further comprising:
if the CARS images indicate that the tissue comprises malignant breast cancer cells, then removing at least a portion of the malignant breast cancer cells using RF ablation.

82. A method for diagnosing and treating a patient, comprising:
obtaining one or more CARS images of tissue within the patient; and
as a function of one or more attributes of the CARS images, determining if the tissue comprises nerve cells.

83. The method of claim 82, further comprising:
if the CARS images indicate that the tissue is identified as comprising nerve cells, then removing other tissue during a surgical procedure.
